(19) United States
(12) Patent Application Publication
(10) Pub. No.: US 2026/0226143 A1
(43) Pub. Date: Aug. 6, 2026

(54) Methods of Treating Neurological Disorders with Anti-Abeta Antibodies

(71) Applicant: Othair Prothena Limited, Dublin 2 (IE)

(72) Inventors: Brian Michael Campbell, El Granada, CA (US); Chad James Swanson, Caldwell, NJ (US); Wagner Zago, San Carlos, CA (US)

(21) Appl. No.: 19/150,987

(22) PCT Filed: Jan. 26, 2024
(86) PCT No.: PCT/IB2024/000067

§ 371 Date: Jul. 25, 2025

Related U.S. Application Data
(60) Provisional application No. 63/481,631, filed on Jan. 26, 2023.
(60) Provisional application No. 63/597,868, filed on Nov. 10, 2023.
(60) Provisional application No. 63/597,861, filed on Nov. 10, 2023.
(60) Provisional application No. 63/618,045, filed on Jan. 5, 2024.

(51) Int. Cl. C07K 16/18; A61K 39/00; A61P 25/28; G01N 33/68

(52) U.S. Cl. CPC C07K 16/18; A61P 25/28; G01N 33/6896; A61K 2039/505; A61K 2039/54; A61K 2039/545; C07K 2317/24; C07K 2317/34; C07K 2317/52; C07K 2317/565; C07K 2317/92; G01N 2333/4709; G01N 2800/2821; G01N 2800/50

(57) ABSTRACT

Antibodies that bind human beta-amyloid peptide, methods of detecting, measuring and treating amyloidogenic disorders with said antibodies, pharmaceutical compositions comprising the antibodies and methods of manufacture are provided.

### CROSS-REFERENCE TO RELATED APPLICATIONS

This application claims the benefit of U.S. Provisional Application Ser. No. 63/481,631, filed Jan. 26, 2023, U.S. Provisional Application Ser. No. 63/597,868, filed Nov. 10, 2023, U.S. Provisional Application Ser. No. 63/597,861, filed Nov. 10, 2023, and U.S. Provisional Application Ser. No. 63/618,045, filed Jan. 5, 2024, each of which is incorporated by reference herein in its entirety.

### SEQUENCE LISTING

A computer readable form of the Sequence Listing is filed with this application by electronic submission and is incorporated into this application by reference in its entirety. The Sequence Listing is contained in the file created on Jan. 4, 2024, having the file name “20-1030-WO2_SeqList” and is 124,150 bytes in size.

### FIELD

The present disclosure relates to anti-Amyloid beta (AB) antibodies as well as compositions and methods of their use.

### BACKGROUND

Alzheimer's disease (AD) is a progressive disease resulting in senile dementia. The disease is generally categorized as late onset, which occurs in old age (65+years) and early onset, which develops well before the senile period, i.e., between 35 and 60 years. Disease pathology appears to be the same for both types of disease, but abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil up to 150 μm across with extracellular amyloid deposits at the center which are visible by microscopic analysis of sections of brain tissue. The accumulation of amyloid plaques within the brain is also associated with Down's syndrome and other cognitive disorders.

The principal constituent of the plaques is a peptide termed amyloid beta (Aβ or Abeta) or β-amyloid peptide. Aβ peptide is a 4-kDa internal fragment of 39-43 amino acids of a larger transmembrane glycoprotein termed amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize Alzheimer's disease.

Monoclonal antibodies (mAbs) targeting amyloid beta have been demonstrated clinically to reduce amyloid plaque burden in patients. The FDA has granted accelerated approval for the anti-amyloid β antibodies aducanumab and lecanemab. The FDA approvals of these antibodies were based on the antibodies demonstrating a reduction of amyloid beta on PET imaging, a surrogate endpoint that was determined to be reasonably likely to predict clinical benefit. Indeed, clinical trials of anti-amyloid β antibodies antibodies, including aducanumab and lecanemab, showed that reduction in plaque burden was associated with slowing of cognitive decline in Alzheimer's disease. However, these treatments suffer from limited efficacy and/or treatment-related side effects. Further, these antibodies require intravenous administration and/or frequent high-dose subcutaneous administration, leading to a burden on patients and caregivers. Therefore, there remains a need for efficacious and safe anti-amyloid β antibodies for the treatment Alzheimer's disease, particularly those that are efficacious when subcutaneously administered as part of a relatively infrequent dosing regimen.

### SUMMARY

The present disclosure relates to antibodies (and antibody fragments) that specifically bind to Aβ, methods of producing such antibodies and antibody fragments and associated nucleic acids, methods of treatment of patients with Aβ-related neurological disorders, pharmaceutical formulations and compositions of antibodies that show high affinity binding to Aβ for prophylactic and/or therapeutic use to, for example, treat, reduce the risk of or delay the outset of amyloidogenic disease, prevent, reduce or inhibit markers of amyloidogenic disease, e.g., amyloid plaques, and improve cognition. The present disclosure further relates to methods of detecting amyloid plaques and measuring the efficacy of treatment in patients being treated for amyloidogenic disease. The disclosure is based, at least in part, on the identification and characterization of monoclonal antibodies that specifically bind to Aβ peptide and are effective at reducing plaque burden and neutralizing soluble Aβ species associated with amyloidogenic disorders.

In a first aspect, the present disclosure provides a method of treating Alzheimer's disease in a subject in need thereof. In one aspect, the present disclosure provides a method of treating Alzheimer's disease in a subject, comprising administering to the subject about 20 mg to about 200 mg of an anti-amyloid β antibody or an antigen-binding fragment thereof once about every 3-5 weeks. In another aspect, the present disclosure provides a method of reducing amyloid plaque in a subject, comprising administering to the subject about 20 mg to about 200 mg of an anti-amyloid β antibody or an antigen-binding fragment thereof once about every 3-5 weeks. In another aspect, the present disclosure provides a method of converting a subject from amyloid positive to amyloid negative, comprising administering to the subject about 20 mg to about 200 mg of an anti-amyloid β antibody or an antigen-binding fragment thereof once about every 3-5 weeks. The method includes administering to the subject about 65 mg to about 200 mg of an antibody or an antigen-binding fragment thereof about once every 3-5 weeks.

In one aspect, the present disclosure provides a method of treating Alzheimer's disease in a subject, comprising administering to the subject about 20 mg to about 200 mg of an anti-amyloid β antibody or an antigen-binding fragment thereof once about every 4 weeks. In another aspect, the present disclosure provides a method of reducing amyloid plaque in a subject, comprising administering to the subject about 20 mg to about 200 mg of an anti-amyloid β antibody or an antigen-binding fragment thereof once about every 4 weeks. In another aspect, the present disclosure provides a method of converting a subject from amyloid positive to amyloid negative, comprising administering to the subject about 20 mg to about 200 mg of an anti-amyloid β antibody or an antigen-binding fragment thereof once about every 4 weeks.

In some embodiments, the anti-amyloid β antibody or an antigen-binding fragment thereof binds to an epitope located within the N-terminus of an Aβ peptide, and the epitope includes at least one amino acid selected from amino acids 1-10 of the Aβ peptide. In some embodiments, the anti-amyloid β antibody or an antigen-binding fragment thereof binds to an epitope comprising at least one amino acid selected from amino acids 1-7 of the AB peptide.

In some embodiments, the anti-amyloid β antibody or an antigen-binding fragment thereof binds to amyloid β₁₋₄₂ protofibrils with an apparent KD of about 5 nM or less. In some embodiments, the anti-amyloid β antibody or an antigen-binding fragment thereof binds to amyloid β₁₋₄₂ protofibrils with an apparent KD of about 1 nM or less. In some embodiments, the anti-amyloid β antibody or an antigen-binding fragment thereof binds to amyloid β₁₋₂₈ monomers with an apparent KD of about 10 nM or less.

In some embodiments, the method comprises administering about 20 mg to about 100 mg of the anti-amyloid β antibody or antigen-binding fragment thereof. In some embodiments, the method comprises administering about 100 mg to about 200 mg of the anti-amyloid β antibody or antigen-binding fragment thereof. In some embodiments, the method comprises administering about 45 mg of the anti-amyloid β antibody or antigen-binding fragment thereof. In some embodiments, the method comprises administering about 70 mg of the anti-amyloid β antibody or antigen-binding fragment thereof. In some embodiments, the method comprises administering about 200 mg of the anti-amyloid β antibody or antigen-binding fragment thereof. In some embodiments, the anti-amyloid β antibody is administered once about every 4 weeks.

In some embodiments, the anti-amyloid β antibody or antigen-binding fragment thereof is administered as a pharmaceutical composition comprising the anti-amyloid β antibody or antigen-binding fragment thereof and a pharmaceutically acceptable diluent. In some embodiments, the pharmaceutically effective amount of the anti-amyloid β antibody or antigen-binding fragment thereof comprises about 45 mg. In some embodiments, the pharmaceutically effective amount of the anti-amyloid β antibody or antigen-binding fragment thereof comprises about 70 mg. In some embodiments, the pharmaceutically effective amount of the anti-amyloid β antibody or antigen-binding fragment thereof comprises about 200 mg. In some embodiments, the administration is once about every 4 weeks.

In some embodiments, the administration is intravenous or subcutaneous. In some embodiments, the administration is subcutaneous.

In some embodiments, the anti-amyloid β antibody or antigen-binding fragment thereof comprises a heavy chain variable region comprising heavy chain CDR1, CDR2, and CDR3 and a light chain variable region comprising light chain CDR1, CDR2, and CDR3, wherein
heavy chain CDR1 comprises the amino acid sequence of one of SEQ ID NO: 16, 19, or 20,
heavy chain CDR2 comprises the amino acid sequence of one of SEQ ID NO: 20, 21, 22, or 23,
heavy chain CDR3 comprises the amino acid sequence of one of SEQ ID NO: 18, 24, or 25,
light chain CDR1 comprises the amino acid sequence of one of SEQ ID NO: 26, 29, 31, or 32,
light chain CDR2 comprises the amino acid sequence of one of SEQ ID NO: 33, 34, 35, or 36, and
light chain CDR3 comprises the amino acid sequence of one of SEQ ID NO: 28, 38, or 39.

In some embodiments, the anti-amyloid β antibody or antigen-binding fragment thereof comprises
heavy chain CDR1 comprises the amino acid sequence of SEQ ID NO: 16,
heavy chain CDR2 comprises the amino acid sequence of SEQ ID NO: 20,
heavy chain CDR3 comprises the amino acid sequence of SEQ ID NO: 18,
light chain CDR1 comprises the amino acid sequence of SEQ ID NO: 29,
light chain CDR2 comprises the amino acid sequence of SEQ ID NO: 34, and
light chain CDR3 comprises the amino acid sequence of SEQ ID NO: 38.

In some embodiments, the heavy chain variable region, excluding the CDRs, is at least 98% identical to the amino acid sequence of SEQ ID NO: 3, and the light chain variable region, excluding the CDRs, is at least 98% identical to the amino acid sequence of SEQ ID NO: 9. In one embodiment of the first aspect, the heavy chain variable region comprises the amino acid sequence of SEQ ID NO: 3, and wherein the light chain variable region comprises the amino acid sequence of SEQ ID NO: 9.

In some embodiments, the anti-amyloid β antibody or antigen-binding fragment thereof comprises
heavy chain CDR1 comprises the amino acid sequence of SEQ ID NO: 16,
heavy chain CDR2 comprises the amino acid sequence of SEQ ID NO: 20,
heavy chain CDR3 comprises the amino acid sequence of SEQ ID NO: 18,
light chain CDR1 comprises the amino acid sequence of SEQ ID NO: 29,
light chain CDR2 comprises the amino acid sequence of SEQ ID NO: 33, and
light chain CDR3 comprises the amino acid sequence of SEQ ID NO: 28.

In some embodiments, the heavy chain variable region, excluding the CDRs, is at least 98% identical to the amino acid sequence of SEQ ID NO: 3, and the light chain variable region, excluding the CDRs, is at least 98% identical to the amino acid sequence of SEQ ID NO: 8. In some embodiments, the heavy chain variable region comprises the amino acid sequence of SEQ ID NO: 3, and wherein the light chain variable region comprises the amino acid sequence of SEQ ID NO: 8.

In some embodiments, the anti-amyloid β antibody or antigen-binding fragment thereof comprises
heavy chain CDR1 comprises the amino acid sequence of SEQ ID NO: 19,
heavy chain CDR2 comprises the amino acid sequence of SEQ ID NO: 21,
heavy chain CDR3 comprises the amino acid sequence of SEQ ID NO: 24,
light chain CDR1 comprises the amino acid sequence of SEQ ID NO: 29,
light chain CDR2 comprises the amino acid sequence of SEQ ID NO: 34, and
light chain CDR3 comprises the amino acid sequence of SEQ ID NO: 38.

In some embodiments, the heavy chain variable region, excluding the CDRs, is at least 98% identical to the amino acid sequence of SEQ ID NO: 4, and the light chain variable region, excluding the CDRs, is at least 98% identical to the amino acid sequence of SEQ ID NO: 9. In some embodiments, the heavy chain variable region comprises the amino acid sequence of SEQ ID NO: 4, and wherein the light chain variable region comprises the amino acid sequence of SEQ ID NO: 9.

In some embodiments, the anti-amyloid β antibody or antigen-binding fragment thereof comprises
heavy chain CDR1 comprises the amino acid sequence of SEQ ID NO: 19,
heavy chain CDR2 comprises the amino acid sequence of SEQ ID NO: 21,
heavy chain CDR3 comprises the amino acid sequence of SEQ ID NO: 25,
light chain CDR1 comprises the amino acid sequence of SEQ ID NO: 29,
light chain CDR2 comprises the amino acid sequence of SEQ ID NO: 34, and
light chain CDR3 comprises the amino acid sequence of SEQ ID NO: 38.

In some embodiments, the heavy chain variable region, excluding the CDRs, is at least 98% identical to the amino acid sequence of SEQ ID NO: 5, and the light chain variable region, excluding the CDRs, is at least 98% identical to the amino acid sequence of SEQ ID NO: 9. In some embodiments, the heavy chain variable region comprises the amino acid sequence of SEQ ID NO: 5, and wherein the light chain variable region comprises the amino acid sequence of SEQ ID NO: 9.

In some embodiments, the anti-amyloid β antibody is a humanized IgG1. In one embodiment of the first aspect, the anti-amyloid β antibody is a full antibody, a chimeric antibody, a CDR-grafted antibody, or a recombinant antibody.

In some embodiments, the anti-amyloid β antibody or antigen-binding fragment thereof further comprises a heavy chain constant region comprising an amino acid sequence at least 95% identical to SEQ ID NO: 40 and/or a light chain constant region comprising an amino acid sequence at least 95% identical to SEQ ID NO: 41.

In some embodiments, the anti-amyloid β antibody comprises a heavy chain of SEQ ID NO: 101, with or without the C-terminal lysine, and a light chain of SEQ ID NO: 102. In some embodiments, the anti-amyloid β antibody is h2731.

Thus, in various aspects, the disclosure are directed to methods utilizing antibodies or fragments thereof that that specifically binds to Aβ peptide. In some embodiments, antibodies and fragments include a heavy chain variable region including heavy chain CDR1, CDR2 and CDR3 and a light chain variable region including light chain CDR1, CDR2 and CDR3, wherein the heavy chain CDR1, CDR2 and CDR3 and the light chain CDR1, CDR2 and CDR3 are as shown for one of the antibodies in Table 1. In addition, the antibodies or fragments or fragments of the disclosure may have a heavy chain variable region that is as shown for one of the antibodies in Table 1 and may have a light chain variable region that is shown for one of the antibodies in Table 1.

In another aspect, the present disclosure provides a method of treating Alzheimer's disease in a subject, the method comprising subcutaneously administering to the subject about 20 mg to about 200 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of treating Alzheimer's disease in a subject, the method comprising subcutaneously administering to the subject about 45 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of treating Alzheimer's disease in a subject, the method comprising subcutaneously administering to the subject about 70 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of treating Alzheimer's disease in a subject, the method comprising subcutaneously administering to the subject about 200 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102.

In another aspect, the present disclosure provides a method of reducing amyloid plaque in a subject, the method comprising subcutaneously administering to the subject about 20 mg to about 200 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of reducing amyloid plaque in a subject, the method comprising subcutaneously administering to the subject about 45 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of reducing amyloid plaque in a subject, the method comprising subcutaneously administering to the subject about 70 mg of an anti-amyloid β antibody once about every 4 weeks; the anti-amyloid β antibody comprising a heavy chain of SEQ ID NO: 101, with or without the C-terminal lysine, and a light chain of SEQ ID NO: 102. In another aspect, the present disclosure provides a method of reducing amyloid plaque in a subject, the method comprising subcutaneously administering to the subject about 200 mg of an anti-amyloid β antibody once about every 4 weeks; the anti-amyloid B antibody comprising a heavy chain of SEQ ID NO: 101, with or without the C-terminal lysine, and a light chain of SEQ ID NO: 102

In another aspect, the present disclosure provides a method of treating Alzheimer's disease in a subject, comprising subcutaneously administering to the subject a dose of an anti-AB antibody sufficient to achieve a Cₐᵥₑ value of about 20 μg/mL to about 40 μg/mL, the anti-AB antibody comprising a heavy chain of SEQ ID NO: 101, with or without the C-terminal lysine, and a light chain of SEQ ID NO: 102.

In another aspect, the present disclosure provides a method of treating Alzheimer's disease in a subject, comprising subcutaneously administering to the subject a dose of an anti-Aβ antibody sufficient to achieve an AUC₀₋ₜₐᵤ value of about 15,000 hr*ug/mL to about 30,000 hr*ug/mL, the anti-Aβ antibody comprising a heavy chain of SEQ ID NO: 101, with or without the C-terminal lysine, and a light chain of SEQ ID NO: 102.

In some embodiments, a maximum concentration over a dosing interval (Cₘₐₓ) of the anti-amyloid β antibody or antigen binding fragment thereof in the subject is about 30 μg/mL to about 60 μg/mL. In some embodiments, a Cₘₐₓ value of the anti-amyloid β antibody or antigen binding fragment thereof in the subject is about 50 μg/mL to about 60 μg/mL. In some embodiments, a Cₘₐₓ value of the anti-amyloid β antibody or antigen binding fragment thereof in the subject does not exceed about 60 μg/mL. In some embodiments, the Cₘₐₓ value is a serum Cₘₐₓ value. In some embodiments, the Cₘₐₓ value is a plasma Cₘₐₓ value.

In some embodiments, an average concentration over the dosing interval (Cₐᵥₑ value) of the anti-amyloid β antibody or antigen binding fragment thereof in the subject is about 20 μg/mL to about 40 μg/mL. In some embodiments, a Cₐᵥₑ value of the anti-amyloid β antibody or antigen binding fragment thereof in the subject is about 30 μg/mL to about 40 μg/mL. In some embodiments, a Cₐᵥₑ value of the anti-amyloid β antibody or antigen binding fragment thereof in the subject does not exceed about 40 μg/mL. In some embodiments, the Cₐᵥₑ value is a serum Cₐᵥₑ value. In some embodiments, the Cₐᵥₑ value is a plasma Cₐᵥₑ value.

In some embodiments, the area under the concentration-time curve for dosing interval (AUC₀₋ₜₐᵤ value) of the anti-amyloid β antibody or antigen binding fragment thereof in the subject is about 15,000 hr*ug/mL to about 30,000 hr*ug/mL. In some embodiments, the AUC₀₋ₜₐᵤ value of the anti-amyloid β antibody or antigen binding fragment thereof in the subject is about 20,000 hr*ug/mL to about 30,000 hr*ug/mL. In some embodiments, the AUC₀₋ₜₐᵤ value of the anti-amyloid β antibody or antigen binding fragment thereof in the subject is does not exceed about 30,000 hr*ug/mL. In some embodiments, the AUC₀₋ₜₐᵤ value is a serum AUC₀₋ₜₐᵤ value. In some embodiments, the AUC₀₋ₜₐᵤ value is a plasma AUC₀₋ₜₐᵤ value.

In some embodiments, amyloid plaque (i.e., brain amyloid beta plaque) in the subject is reduced. In some embodiments, the method further comprises reducing amyloid plaques in the subject.

In some embodiments, the reduction of brain amyloid beta plaque comprises a reduction of at least about 30 centiloids to about 70 centiloids. In some embodiments, the reduction of brain amyloid beta plaque comprises a reduction of from about 45 centiloids to about 80 centiloids. In some embodiments, the reduction of brain amyloid beta plaque comprises a reduction of about 50 centiloids to about 85 centiloids.

In some embodiments, the reduction of brain amyloid beta plaque comprises a reduction of at least about 40% to about 90%. In some embodiments, the reduction of brain amyloid beta plaque comprises a reduction of from about 60% to about 100%. In some embodiments, the reduction of brain amyloid beta plaque comprises a reduction of about 65% to about 100%.

In some embodiments, the reduction of brain amyloid plaque is a reduction compared to baseline (e.g., a value prior to treatment). In some embodiments, the reduction of brain amyloid beta plaque is a reduction compared to the subject prior to the administration of the anti-amyloid β antibody.

In some embodiments, the reduction of brain amyloid beta plaque is achieved after 6 months of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction of brain amyloid beta plaque is assessed by Positron Emission Tomography (PET).

In some embodiments, the subject is converted from amyloid positive to amyloid negative. In some embodiments, treating comprises increasing a probability of converting the subject from amyloid positive to amyloid negative.

In some embodiments, treating comprises a probability of converting the subject from amyloid positive to amyloid negative by about 10% to about 40%. In some embodiments, treating comprises a probability of converting the subject from amyloid positive to amyloid negative by about 30% to about 60%. In some embodiments, treating comprises a probability of converting the subject from amyloid positive to amyloid negative by about 40% to about 80%.

In some embodiments, the probability of converting the subject from amyloid positive to amyloid negative is a probability after about 6 months of treatment. In some embodiments, the probability of converting the subject from amyloid positive to amyloid negative is a probability after about 12 months of treatment. In some embodiments, the probability of converting the subject from amyloid positive to amyloid negative is a probability after about 18 months of treatment.

In some embodiments, treating comprises slowing, halting, or reversing decline in cognitive function. In some embodiments, treating comprises slowing decline in cognitive function. In some embodiments, cognitive function is measured by at least one of the following CRD-SB, ADAS-Cog14, ADCOMS, and ADCS MCI-ADL. In some embodiments, cognitive function is measured by ADCOMS

In another aspect, the present disclosure provides a method of modulating a biomarker in a subject, comprising administering to the subject from about 20 mg to about 200 mg of an anti-amyloid β antibody once about every 3-5 weeks, the anti-amyloid β antibody comprising a heavy chain of SEQ ID NO: 101, with or without the C-terminal lysine, and a light chain of SEQ ID NO: 102. In another aspect, the present disclosure provides a method of increasing a ratio of Aβ 42/40 in a subject, comprising administering to the subject about 20 mg to about 200 mg of an anti-amyloid β antibody once about every 3-5 weeks, the anti-amyloid β antibody comprising a heavy chain of SEQ ID NO: 101, with or without the C-terminal lysine, and a light chain of SEQ ID NO: 102. In another aspect, the present disclosure provides a method of decreasing an amount of phospho-tau in a subject, comprising administering to the subject about 20 mg to about 200 mg of an anti-amyloid β antibody thereof once about every 3-5 weeks, the anti-amyloid β antibody comprising a heavy chain of SEQ ID NO: 101, with or without the C-terminal lysine, and a light chain of SEQ ID NO: 102.

In some embodiments, the administration is intravenous or subcutaneous. In some embodiments, the administration is a subcutaneous injection. In some embodiments, the administration is a single subcutaneous injection.

In some embodiments, a biomarker in the subject is modulated. In some embodiments, the biomarker in the subject is modulated compared to baseline. In some embodiments, the method further comprises detecting a biomarker in a sample collected from the subject. In some embodiments, the method further comprises quantifying a biomarker in a sample collected from the subject.

In some embodiments, the biomarker comprises the ratio of Aβ 42/40 in the subject. In some embodiments, the ratio of Aβ 42/40 in the subject. In some embodiments, the ratio of Aβ 42/40 in the subject increases at least 25%. In some embodiments, the ratio of Aβ 42/40 in the subject increases at least 50%. In some embodiments, the ratio of Aβ 42/40 in the subject increases about 25% to about 100%. In some embodiments, the ratio of Aβ 42/40 in the subject increases about 50% to about 100%.

In some embodiments, the biomarker comprises a phospho-tau value. In some embodiments, the phospho-tau value comprises at least one of the following: a p181-tau value, a p212-tau value, p217-tau value, a p231-tau value, and a p235-tau value. In some embodiments, the phospho-tau value comprises a p181-tau value. In some embodiments, the phospho-tau value comprises a p212-tau value. In some embodiments, the phospho-tau value comprises a p217-tau value. In some embodiments, the phospho-tau value comprises a p231-tau value. In some embodiments, the phospho-tau value comprises a p235-tau value. In some embodiments, the phospho-tau value decreases. In some embodiments, the phospho-tau value decreases at least about 10%. In some embodiments, the phospho-tau value decreases about 10% to about 30%. In some embodiments, the phospho-tau value decreases about 20% to about 30%.

In some embodiments, the sample comprises blood or a portion thereof collected from the subject. In some embodiments, the sample comprises plasma collected from the subject. In some embodiments, the sample comprises serum collected from the subject. In some embodiments, the sample comprises cerebral spinal fluid (“CSF”) collected from the subject.

In some embodiments, the method comprises a risk of ARIA-E that is less than about 45%. In some embodiments, the method comprises a risk of ARIA-E from about 25% to about 45%. In some embodiments, the method comprises a risk of ARIA-E of less than about 75%. In some embodiments, the method comprises a risk of ARIA-E of about 50% to about 75%. In some embodiments, the method comprises a risk of symptomatic ARIA-E that is less than about 15%. In some embodiments, the method comprises a risk of symptomatic ARIA-E that is less than about 30%. In some embodiments, the risk of ARIA-E is a risk of severe ARIA-E. In some embodiments, the risk of ARIA-E is the risk after about 6 months of treatment. In some embodiments, the risk of ARIA-E is the risk after about 12 months of treatment. In some embodiments, the risk of ARIA-E is the risk after about 18 months of treatment. In some embodiments, the subject does not experience symptomatic ARIA-E during treatment.

In some embodiments, the method comprises a risk of ARIA-H that is less than about 35%. In some embodiments, the method comprises a risk of ARIA-H from about 10% to about 35%. In some embodiments, the risk of ARIA-H is a risk of severe ARIA-H. In some embodiments, the risk of ARIA-H is the risk after about 6 months of treatment. In some embodiments, the subject does not experience symptomatic ARIA-H during treatment.

In some embodiments, ARIA is assessed by Magnetic Resonance Imagining (“MRI”).

In some embodiments, the subject is an APOE4 homozygous subject. In some embodiments, the subject is an APOE4 heterozygous subject or an APOE4 noncarrier. In some embodiments, the method further comprises determining the APOE4 status of the subject prior to administration.

In some embodiments, the duration of the treatment is at least 6 months. In some embodiments, the duration of the treatment is at least 12 months. In some embodiments, the duration of the treatment is at least 18 months.

In some embodiments, the administration is performed using a syringe. In some embodiments, the administration is performed using an autoinjector.

In some embodiments, the subject is a mammal. In some embodiments, the subject is a human.

In various aspects, the disclosure is directed to pharmaceutical compositions comprising the anti-amyloid β antibody or an antigen-binding fragment as described herein for treating Alzheimer's disease in a subject. In the various aspects, the treatment includes administering to the subject about 20 mg to about 200 mg of the antibody or antigen binding fragment thereof once about every 3-5 weeks. In aspects, the intermediate doses as described herein can be used for subcutaneous administration. In embodiments, the pharmaceutical compositions include pharmaceutically acceptable excipients for administration, including, for example, subcutaneous administration.

In various aspects, the disclosure is directed to pharmaceutical compositions comprising an anti-amyloid β antibody or an antigen-binding fragment for reducing amyloid plaque in a subject. In the various aspects, the treatment of the subject includes administering to the subject about 20 mg to about 200 mg of an anti-amyloid β antibody or the antigen-binding fragment thereof once about every 3-5 weeks. In aspects, the intermediate doses as described herein can be used for subcutaneous administration. In embodiments, the pharmaceutical compositions include pharmaceutically acceptable excipients for administration, including, for example, subcutaneous administration.

In various aspects, the disclosure is directed to pharmaceutical compositions comprising an anti-amyloid β antibody or an antigen-binding fragment as described herein for converting a subject from amyloid positive to amyloid negative. In the various aspects, treatment of the subject includes administering to the subject about 20 mg to about 200 mg of the anti-amyloid β antibody or the antigen-binding fragment thereof once about every 3-5 weeks. In aspects, the intermediate doses as described herein can be used for subcutaneous administration. In embodiments, the pharmaceutical compositions include pharmaceutically acceptable excipients for administration, including, for example, subcutaneous administration.

In embodiments of various pharmaceutical compositions, the administration includes, for example, subcutaneously administering to the subject about 45 mg of an anti-amyloid β antibody once about every 4 weeks, subcutaneously administering to the subject about 70 mg of an anti-amyloid β antibody once about every 4 weeks, or subcutaneously administering to the subject about 200 mg of an anti-amyloid β antibody once about every 4 weeks.

In various aspects, the disclosure is directed to the use of an anti-amyloid β antibody or an antigen-binding fragment as described herein for the manufacture of a medicament for treating Alzheimer's disease in a subject. In various aspects, the medicament is for administration to the subject at about 20 mg to about 200 mg of the anti-amyloid β antibody or an antigen-binding fragment once about every 3-5 weeks. In aspects, the intermediate doses as described herein can be used for subcutaneous administration.

In various aspects, the disclosure is directed to the use of an anti-amyloid β antibody or an antigen-binding fragment as described herein for the manufacture of a medicament for reducing amyloid plaque in a subject. In various aspects, the medicament is for administration to the subject about 20 mg to about 200 mg of the anti-amyloid β antibody or the antigen-binding fragment thereof once about every 3-5 weeks. In aspects, the intermediate doses as described herein can be used for subcutaneous administration.

In various aspects, the disclosure is directed to the use of an anti-amyloid β antibody or an antigen-binding fragment as described herein for the manufacture of a medicament for converting a subject from amyloid positive to amyloid negative. In the various aspects, the medicament is for administration to the subject to the subject at about 20 mg to about 200 mg of the anti-amyloid β antibody or the antigen-binding fragment thereof once about every 3-5 weeks. In aspects, the intermediate doses as described herein can be used for subcutaneous administration.

In embodiments of the uses of the anti-amyloid β antibody or the antigen-binding fragment thereof, the administration includes, for example, subcutaneously administering to the subject about 45 mg of an anti-amyloid β antibody or binding fragment once about every 4 weeks, subcutaneously administering to the subject about 70 mg of an anti-amyloid β antibody or binding fragment once about every 4 weeks, or subcutaneously administering to the subject about 200 mg of an anti-amyloid β antibody or binding fragment once about every 4 weeks.

### BRIEF DESCRIPTION OF THE DRAWINGS

FIG. 1 shows an alignment of three different versions of VL that were designed by incorporating human germline framework residues into bapineuzumab (hBP) VL sequence. Canonical or interface residues were not changed.

FIG. 2 shows competitive ELISA assay graphs for 4918, 4917, 4921, 3818, 49human3, 2931 and bapineuzumab control for IC₅₀ ratio determination relative to bapineuzumab (hBP).

FIG. 3 shows competitive ELISA assay graphs for 2926, 2831, 2927, 2726, 2731, 2826 and bapineuzumab control for IC₅₀ ratio determination relative to bapineuzumab (hBP).

FIG. 4 shows competitive ELISA assay graphs for 2727, 2931 and bapineuzumab control for IC₅₀ ratio determination relative to bapineuzumab (hBP).

FIG. 5A and FIG. 5B show competitive ELISA assay graphs for 2931, 2731 and bapineuzumab (FIG. 5A) and 2726, 2831 and bapineuzumab (FIG. 5B).

FIGS. 6A-6D show BIAcore sensorgrams of binding of h2726 (FIG. 6A), h2731 (FIG. 6B), h2831 (FIG. 6C) and 2931 (FIG. 6D) to Aβ₁₋₂₈ at analyte concentrations from 100 nM to 0.39 nM (2-fold serial dilutions).

FIG. 7 shows a BIAcore sensorgram comparing binding characteristics of humanized antibodies (PB-0569 (aducanumab), PB-0573 (h2726), PB-0574 (h2731), PB-0575 (h2831), PB-0576 (h2931)) to recombinant Abeta 1-42 (Aβ₁₋₄₂) fibrils.

FIG. 8 shows h2931 binds soluble Aβ oligomers with high relative affinity.

FIG. 9 shows graphs evaluating Aβ fibril binding activity of 2726, 2731, 2831, 2931 versus aducanumab control. Antibody was titrated in a constant concentration of Aβ fibrils (left panel) or Aβ fibrils were titrated in a constant concentration of antibody (right panel), both indicating substantially better binding for 2726, 2731, 2831 and 2931 than for aducanumab.

FIG. 10 shows Aβ binding in AD brain. Binding to tissue Aβ pathology appears similar among h2726, h2731, h2831 and h2931 antibodies. Examples of images stained with the four antibodies, h2726, h2731, h2831, h2931, at 0.3 μg/ml show their pattern of staining in two AD brains with different amounts of Aβ pathology (AD 11-97 and AD 13-75). For each brain, the images are from the same area of the section and show comparatively similar intensity and distribution of pathology with all four antibodies. Staining with aducanumab was always the weakest (Scale bar: 500 μm).

FIG. 11 shows Aβ binding in AD brain of controls. Human IgG isotype control antibody produced no staining in AD brains. As shown in these examples, AD sections incubated with human IgG isotype at 1 μg/ml were devoid of any staining (Scale bar: 500 μm).

FIG. 12 shows quantification of Aβ binding in AD brain. Quantification of Aβ pathology staining in AD tissues revealed similar binding between h2726, h2731, h2831 and h2931 antibodies. Section from four AD brains were incubated with the antibodies h2726, h2731, h2831, h2931 as well as aducanumab at the following concentrations: 0.03, 0.1, 0.3, 1, 3 and 9 μg/ml. After imaging of sections, the percent of stained tissue area was determined morphometrically using Halo® imaging analysis software. Each graph compares measurements in an AD brain obtained with the five antibodies. The four graphs consistently show that the binding profiles of h2726, h2731, h2831, h2931 antibodies are similar. Measurements obtained with aducanumab were significantly lower.

FIG. 13 shows Aβ binding in AD brain. hBP binds to tissue Aβ pathology strongly and in a dose-dependent manner. Images from relatively the same area of the section (Brain AD 13-75) with similar pathology distribution. hBP shows an increase in the amount of staining with concentration, and its binding to Aβ pathology was stronger than that of BAN2401 or aducanumab at each concentration (Scale bar: 500 μm).

FIGS. 14A and 14B show individual (FIG. 14A) and pooled (FIG. 14B) results from an ex vivo phagocytosis study of h2931 and aducanumab in APP.PS1 Tg mouse tissue with primary murine microglia. h2931 and aducanumab both demonstrate highly significant reductions in Aβ₁₋₄₂ over isotype control.

FIGS. 15A and 15B show graphs indicating a reduction of soluble oligomer binding to neurites on rat hippocampal neurons with increasing concentration of h2726, h2731, h2831 and h2931 compared to isotype control, and normalized by +/−Aβ addition. FIG. 15A shows spots per neuron and FIG. 15B shows total spot counts (at 40 fields per well).

FIG. 16 shows a graph representing the percentage of Aβ spots per neuron with increasing concentration of 2726, 2731, 2831 and 2931 normalized by +/−Aβ addition.

FIG. 17 shows an alignment of bapineuzumab variable heavy chain sequence and four sequences of the disclosure, 2726, 2731, 2831 and 2931. CDRs are in bold.

FIG. 18 shows an alignment of bapineuzumab light chain sequence and four (variable light chain) sequences of the disclosure, 2726, 2731, 2831 and 2931. CDRs are in bold.

FIGS. 19A and 19B show a CDR table listing the variable heavy and light chain CDR sequences for antibodies of the disclosure. FIG. 19A refers to heavy chain CDRs and FIG. 19B refers to light chain CDRs.

FIGS. 20A and 20B show graphs measuring antibody potency for binding heterogeneous aggregated Aβ42 species by competition ELISA. FIG. 20A shows h2931, h2731 and bapineuzumab control, and FIG. 20BA shows h2831, h2726 and bapineuzumab control.

FIG. 21 shows graphs measuring direct binding and relative affinity of antibodies to fibrillar Aβ42 by ELISA.

FIG. 22 shows graphs measuring antibody dose response of Aβ plaque area binding measured as percent positive tissue by immunohistochemical staining in AD brain.

FIG. 23 shows quantification of binding of soluble Aβ to rat hippocampal neurons in the presence of antibody.

FIG. 24 shows results from an ex vivo phagocytosis study of h2731 in AD tissue with primary murine microglia. h2731 demonstrated highly significant reduction in Aβ₁₋₄₂ indicating the antibody robustly promoted phagocytosis and removal of these species.

FIGS. 25A and 25B confirm the presence of pyroglutamate-3 Aβ (Aβ_{pE3-42}) in AD tissue used for ex vivo phagocytosis assays (FIG. 23A) and demonstrates a similar binding pattern for pyroglutamate-3 Aβ and h2931 (FIGS. 23A and B).

FIGS. 26A and 26B show results from an ex vivo phagocytosis study of h2931 and h2731 in AD tissue with primary murine microglia. h2931 and h2731 both demonstrate highly significant reductions in pyroglutamate-3 Aβ (Aβ_{pE3-42}) indicating that both antibodies robustly promote phagocytosis and removal of these species.

FIG. 27 shows that h2731 binds the N-terminus of Aβ₁₋₄₂ but not Aβ_{pE3-42}.

FIGS. 28A and 28B show that antibodies of the present invention induce phagocytosis of Aß₁₋₄₂ protofibrils in THP-1 human monocytes in vitro.

FIG. 29A and FIG. 29B show the distribution pattern of Aß_{1-XX}, as measured by an N-terminal anti-Ab antibody, compared to Aß_{pE3-42} in human AD brain tissue. FIG. 29C shows the quantification of the percent area covered by Aß_{1-XX} compared to Aß_{pE3-42} in human AD brain tissue.

FIG. 30 shows localization of h2731 to Aß plaques, localization of anti-Aß_{pE3-42} antibody signal to Aβ plaques, and colocalization of h2731 and anti-Aß_{pE3-42} antibody signal to Aß plaques.

FIG. 31A and FIG. 31B show that anti-Aβ antibody h2731 promotes Aß_{pE3-42} clearance from AD brain tissue ex vivo in a dose-dependent manner with higher potency than aducanumab.

FIG. 32A shows the concentration dependence of h2731 and aducanumab clearance of Aβ_{pE3-42} from AD brain tissue, and FIG. 32B shows that the effect of h2731 is microglia-dependent.

FIG. 33 compares predicted CNS exposure of h2731 and aducanumab with repeated dosing.

FIG. 34 shows that anti-Aβ antibody h2731 promotes clearance of plaques containing Aß_{pE3-42} in AD brain tissue ex vivo.

FIG. 35 is a schematic representation of a clinical trial plan for a single ascending dose study of h2731 in healthy volunteers and Alzheimer's disease subjects. Certain inclusion criteria, dosages, and a schedule of certain assessments are shown.

FIG. 36 is a schematic representation of a clinical trial plan a multiple ascending dose study of h2731 in Alzheimer's disease subjects. Certain inclusion criteria, dosages, and a schedule of certain assessments are shown.

FIG. 37 shows details of an open label extension study of h2731 in some Alzheimer's disease subjects who were enrolled in either the single ascending dose study of Example 19 or the multiple ascending dose study of Example 20. Certain dosages and a schedule of certain assessments are shown.

### DESCRIPTION

Monoclonal antibodies (mAbs) targeting the N-terminus of amyloid beta (Aβ) have been demonstrated clinically to reduce amyloid plaque burden and one such antibody, aducanumab, showed that significant reduction in plaque burden was associated with slowing of cognitive decline in Alzheimer's disease (AD). Preclinical studies have also indicated that monoclonal antibodies (mAbs) targeting N-terminal epitopes of Aβ elicit an antibody-dependent microglial-mediated Aβ-plaque clearance and neutralization of soluble toxic Aβ oligomers both in vitro and in vivo. It is hypothesized that administration of N-terminal Aβ targeting mAbs slows disease progression via clearance of Aβ plaques and neutralization of soluble Aβ aggregates in patients with AD.

Aβ antibody bapineuzumab (hBP) is a humanized antibody developed from parental murine antibody 3D6. In accordance with various aspects of the disclosure, a multipronged approach was applied to construct superior antibodies to hBP. Humanness of hBP was analyzed and a determination was made that light chain humanization could be optimized.

A search was made over the protein sequences in the PDB database [Deshpande et al, 2005] to find structures that would provide a rough structural model of hBP. The crystal structure of hBP fab PDB code 4HIX [Miles, et al., 2013] was utilized for both Vh and Vk structure as it had acceptable resolution and an exact sequence match to hBP Vh and Vk, retaining the same canonical structures for the loops.

IMGT/DomainGapAlignment was performed for the hBP VL as input sequences. to identify human germ line VK gene sequence IGHV2-30*02 as the closest matched to hBP VL. The frameworks of hBP VL share a high degree of sequence similarity with the corresponding framework regions of IGHV2-30*02. Thus, the framework regions of IGHV2-30*02 VL were chosen as the guidance sequence for further optimization of the hBP framework regions. Additional residues in CDR-L2 that do not make any direct contact with the antigen as per hBP 3D structure were also changed to germline sequence resulting in following changes.

Three different versions of VL were designed by incorporating human germline framework residues into hBP VL sequence. Canonical or interface residues were not changed. Also, based on structural observation that P15 is located at a turn and the germline gene has Leu at this position, P15L was tested in one version of the variable light chain.

Based on the 3D structural observations, substitutions at a number of residues in the light chain and heavy chain CDRs and framework were designed. Mutant VL and VH versions were generated and tested for binding in the first round of rational design. Mutations that showed improved binding were combined in the second round of the rational design. Additionally, new mutations guided by further analysis of the structure were also incorporated into the design.

Accordingly, the disclosure provides antibodies (and antibody fragments), nucleic acids encoding and methods of producing such antibodies and antibody fragments, pharmaceutical compositions, and methods for preventing or treating amyloidogenic disease, reducing the risk or delaying the outset of an amyloidogenic disease, effecting improvement of cognition in an subject having a condition related to amyloidogenic disease, inhibiting the formation of Aβ plaque in a subject, reducing Aβ plaque in the brain of a subject, inhibiting or reducing amyloid plaque in a subject at risk of developing an amyloidogenic disease, detecting amyloid plaques, measuring efficacy of a treatment in a subject being treated for an amyloidogenic disease, where amyloidogenic disease comprises Alzheimer's and others as described herein. The disclosure is based, at least in part, on the characterization of a genus of monoclonal antibodies effective at binding beta amyloid protein (Aβ) (e.g., binding soluble and/or aggregated Aβ), mediating phagocytosis (e.g., of aggregated Aβ), reducing plaque burden and/or reducing neuritic dystrophy (e.g., in patient), neutralizing soluble, toxic Aβ species. The antibodies and fragments of the disclosure exhibit greater binding strength (affinity and/or avidity) for pathologic fibrillar Aβ than reported current experimental therapies, and high affinity for soluble toxic Aβ forms. These antibodies may enable more convenient dosing strategies and enhanced patient access.

Before describing particular aspects of the disclosure in more detail, a number of terms are defined.

### Definitions

The term “antibody” includes intact antibodies and binding fragments thereof. Typically, fragments compete with the intact antibody from which they were derived for specific binding to the target. Fragments include separate heavy chains, light chains Fab, Fab′, F(ab′)₂, F (ab) c, Fv and single domain antibodies. Single (variable) domain antibodies include VH regions separated from their VL partners (or vice versa) in conventional antibodies (Ward et al., 1989, Nature 341:544-546) as well as VH regions (sometimes known as VHH) from species such as Camelidae or cartilaginous fish (e.g., a nurse shark) in which VH regions are not associated with VL regions (see, e.g., WO 9404678). Single domain antibodies in which one chain is separated from its natural partners are sometimes known as Dabs and single domain antibodies from Camelidae or cartilaginous fish are sometimes known as nanobodies. Constant regions or parts of constant regions may or may not be present in single domain antibodies. For example, natural single variable region antibodies from Camelidae include a VHH variable region, and CH2 and CH3 constant regions. Single domain antibodies can be subject of humanization by analogous approaches to conventional antibodies. The Dabs type of antibodies are usually obtained from antibodies of human origin. NANOBODY types of antibody are of Camelidae or shark origin and can be subject to humanization. Fragments can be produced by recombinant DNA techniques, or by enzymatic or chemical separation of intact immunoglobulins. The term “antibody” also includes a bispecific antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

An immunoglobulin light or heavy chain variable region (also sometimes referred to herein as a “light chain variable domain” (“VL domain”) or “heavy chain variable domain” (“VH domain”), respectively) consists of a “framework” region interrupted by three “complementarity determining regions” or “CDRs.” The framework regions serve to align the CDRs for specific binding to an epitope of an antigen. The CDRs include the amino acid residues of an antibody that are primarily responsible for antigen binding. From amino-terminus to carboxyl-terminus, both VL and VH domains comprise the following framework (FR) and CDR regions: FR1, CDR1, FR2, CDR2, FR3, CDR3, and FR4. CDRs 1, 2, and 3 of a VL domain are also sometimes referred to herein, respectively, as CDR-L1, CDR-L2, and CDR-L3; CDRs 1, 2, and 3 of a VH domain are also sometimes referred to herein, respectively, as CDR-H1, CDR-H2, and CDR-H3. When the application discloses a VL sequence with R as the C-terminal residue, the R can alternatively be considered as being the N-terminal residue of the light chain constant region. Thus, the application should also be understood as disclosing the VL sequence without the C-terminal R.

The assignment of amino acids to each VL and VH domain is in accordance with any conventional definition of CDRs. Conventional definitions include, the Kabat definition (Kabat, Sequences of Proteins of Immunological Interest (National Institutes of Health, Bethesda, MD, 1987 and 1991), the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al (bioinfo.org.uk/abs) (see Table A). Kabat provides a widely used numbering convention (Kabat numbering) in which corresponding residues between different heavy chains or between different light chains are assigned the same number. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

TABLE A
Conventional Definitions of CDRs Using Kabat Numbering

| | | | Composite of | | |
|---|---|---|---|---|---|
| | | | Chothia & | | |
| Loop | Kabat | Chothia | Kabat | AbM | Contact |
| L1 | L24--L34 | L24--L34 | L24--L34 | L24--L34 | L30--L36 |
| L2 | L50--L56 | L50--L56 | L50--L56 | L50--L56 | L46--L55 |
| L3 | L89--L97 | L89--L97 | L89--L97 | L89--L97 | L89--L96 |
| H1 | H31--H35B | H26-- | H26--H35B* | H26--H35B | H30--H35B |
| | | H32 . . . H34* | | | |
| H2 | H50--H65 | H52--H56 | H50--H65 | H50--H58 | H47--H58 |
| H3 | H95--H102 | H95--H102 | H95--H102 | H95--H102 | H93--H101 |

*CDR-H1 by Chothia can end at H32, H33, or H34 (depending on the length of the loop). This is because the Kabat numbering scheme places insertions of extra residues at 35A and 35B, whereas Chothia numbering places them at 31A and 31B. If neither H35A nor H35B (Kabat numbering) is present, the Chothia CDR-H1 loop ends at H32. If only H35A is present, it ends at H33. If both H35A and H35B are present, it ends at H34.

In some embodiments, the CDRs of the humanized antibodies of the present invention are of a definition selected from the group of Kabat, Chothia, Kabat/Chothia Composite, AbM and Contact.

One or several amino acids at the amino or carboxy terminus of the light and/or heavy chain, such as a C-terminal lysine of the heavy chain, may be missing or derivatized in a portion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduce affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine. In some antibodies, the isotype is human IgG2 or IgG4. As an example, the C-terminal lysine on the antibody heavy chains heavy chain constant regions described herein is optional such that sequences may be considered with or without the C-terminal lysine.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and preferably three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody. “Excluding the CDRs” as used herein means the portions of the antibody that do not include the amino acids of the CDRS, for example the framework regions and antibody constant regions.

Accordingly, regions or residues of a humanized immunoglobulin or antibody, or of a humanized immunoglobulin or antibody chain, except possibly the CDRs, are substantially identical to the corresponding regions or residues of one or more native human immunoglobulin sequences. The term “corresponding region” or “corresponding residue” refers to a region or residue on a second amino acid or nucleotide sequence which occupies the same (i.e., equivalent) position as a region or residue on a first amino acid or nucleotide sequence, when the first and second sequences are optimally aligned for comparison purposes.

The term “epitope” or “antigenic determinant” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. When an epitope is said to be within a range of amino acid residues in a protein (e.g., within residues 1 to 6 of Aβ), the range is inclusive of the residues defining its borders. Certain residues within the range contribute to the epitope, whereas others may not. The residues that form the epitope may or may not be contiguous with one another. Similarly, when an antibody binds to an epitope found within a particular range of amino acids, the antibody need not contact all the amino acids residues within the range, and the residues of the epitope that are contacted by the antibody may or may not be contiguous with one another. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

Antibodies that recognize the same epitope can be identified in a simple immunoassay showing the ability of one antibody to block or compete with the binding of another antibody to a target antigen, i.e., a competitive binding assay. Competitive binding is determined in an assay in which the immunoglobulin under test inhibits specific binding of a reference antibody to a common antigen, such as Aβ. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25 (1): 7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70%, 70-75%, or more.

Competition between antibodies is determined by an assay in which an antibody under test inhibits specific binding of a reference antibody (e.g. 3D6, aducanumab, bapineuzumab) to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

An epitope is also recognized by immunologic cells, for example, B cells and/or T cells. Cellular recognition of an epitope can be determined by in vitro assays that measure antigen-dependent proliferation, as determined by ³H-thymidine incorporation, by cytokine secretion, by antibody secretion, or by antigen-dependent killing (cytotoxic T lymphocyte assay).

Exemplary epitopes or antigenic determinants can be found within the human amyloid precursor protein (APP) but are preferably found within the Aβ peptide of APP. Multiple isoforms of APP exist, for example APP⁶⁹⁵, APP⁷⁵¹, and APP⁷⁷⁰. Amino acids within APP are assigned numbers according to the sequence of the APP₇₇₀ isoform (see e.g., GenBank Accession No. P05067, also set forth as SEQ ID NO: 85).

Aβ (also referred to herein as beta amyloid peptide and A-beta) peptide is a about 4-kDa internal fragment of 39-43 amino acids of APP (Aβ39, Aβ40, Aβ41, Aβ42, and Aβ43). Aβ40, for example, consists of residues 672-711 of APP and Aβ42 consists of residues 673-713 of APP. As a result of proteolytic processing of APP by different secretase enzymes in vivo or in situ, Aβ is found in both a “short form”, 40 amino acids in length, and a “long form”, ranging from 42-43 amino acids in length. Preferred epitopes or antigenic determinants, as described herein, are located within the N-terminus of the Aβ peptide and include residues within amino acids 1-10 of Aβ, preferably from residues 1-3, 1-4, 1-5, 1-6, 1-7, or 3-7 of Aβ42. Additional referred epitopes or antigenic determinants include residues 2-4, 5, 6, 7, or 8 of Aβ, residues 3-5, 6, 7, 8, or 9 of Aβ, or residues 4-7, 8, 9, or 10 of Aβ42.

“Soluble” or “dissociated” Aβ refers to Aβ species that are either monomeric, aggregated, oligomeric, associated or not with other proteins and lipids, which remain in solution (supernatant) after centrifugation at 100,000×g. “Insoluble” Aβ refers to aggregated Aβ species, amyloid (beta-sheet) or not, that do not remain in solution after 100,000×g centrifugation, for example, Aβ held together by noncovalent bonds. Aβ (e.g., Aβ42) is believed to aggregate, at least in part, due to the presence of hydrophobic residues at the C-terminus of the peptide (part of the transmembrane domain of APP). One method to prepare soluble Aβ is to dissolve lyophilized peptide in neat DMSO with sonication. The resulting solution is centrifuged to remove any insoluble particulates.

“Specific binding” of an antibody mean that the antibody exhibits appreciable affinity for antigen or a preferred epitope and, preferably, does not exhibit significant cross reactivity. “Appreciable” or preferred binding include binding with an affinity of at least 10⁶, 10⁷, 10⁸, 10⁹ M⁻¹, or 10¹⁰ M⁻¹. Affinities greater 10⁷ M⁻¹, preferably greater than 10⁸ M⁻¹ are more preferred. Values intermediate of those set forth herein are also intended to be within the scope of the present disclosure and a preferred binding affinity can be indicated as a range of affinities, for example, 10⁶ to 10¹⁰ M⁻¹, preferably 10⁷ to 10¹⁰ M⁻¹, more preferably 10⁸ to 10¹⁰ M⁻¹. An antibody that “does not exhibit significant cross reactivity” is one that will not appreciably bind to an undesirable entity (e.g., an undesirable proteinaceous entity). For example, an antibody that specifically binds to Aβ will appreciably bind Aβ but will not significantly react with non-Aβ proteins or peptides (e.g., non-Aβ proteins or peptides included in plaques). An antibody specific for a preferred epitope will, for example, not significantly cross-react with remote epitopes on the same protein or peptide. Specific binding can be determined according to any art-recognized means for determining such binding. Preferably, specific binding is determined according to Scatchard analysis and/or competitive binding assays.

Binding fragments are produced by recombinant DNA techniques, or by enzymatic or chemical cleavage of intact immunoglobulins. Binding fragments include Fab, Fab′, F(ab′)₂, Fabc, Fv, single chains, and single-chain antibodies.

The term “patient” includes human and other mammalian subjects that receive either prophylactic or therapeutic treatment. In some embodiments, the term “individual” is used interchangeably with “patient.”

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the infection and the general state of the patient's own immune system.

The term “treatment” as used herein, is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease.

The term “amyloidogenic disease” includes any disease associated with (or caused by) the formation or deposition of insoluble amyloid fibrils or amyloid plaques. Exemplary amyloidogenic diseases include, but are not limited to systemic amyloidosis, Alzheimer's disease, mature onset diabetes, Parkinson's disease, Huntington's disease, fronto-temporal dementia, Down's syndrome, mild cognitive impairment, prion-related transmissible spongiform encephalopathies (kuru and Creutzfeldt-Jacob disease in humans and scrapie and BSE in sheep and cattle, respectively), and the like. Different amyloidogenic diseases are defined or characterized by the nature of the polypeptide component of the fibrils deposited. For example, in subjects or patients having Alzheimer's disease, β-amyloid protein (e.g., wild-type, variant, or truncated β-amyloid protein) is the characterizing polypeptide component of the amyloid deposit. Accordingly, Alzheimer's disease is an example of a “disease characterized by deposits of Aβ” or a “disease associated with deposits of Aβ”, e.g., in the brain of a subject or patient. The terms “β-amyloid protein”, “β-amyloid peptide”, “β-amyloid”, “Aβ” and “Aβ peptide” are used interchangeably herein.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor.

The term “symptom” refers to a subjective evidence of a disease, such as altered gait, as perceived by the patient. A “sign” refers to objective evidence of a disease as observed by a physician.

Statistical significance means p<0.05.

“Half-life (t1/2)” refers to the time required for the concentration of the antigen binding polypeptide to reach half of its original value. The serum half-life of proteins can be measured by pharmacokinetic studies according to the method described by Kim et al. (Eur. J. of Immuno. 24:542, 1994). According to this method, radiolabeled protein is injected intravenously into mice and its plasma concentration is periodically measured as a function of time, for example, at about 3 minutes to about 72 hours after the injection. Other methods for pharmacokinetic analysis and determination of the half-life of a molecule will be familiar to those skilled in the art. Details may be found in Kenneth, A et al: Chemical Stability of Pharmaceuticals: A Handbook for Pharmacists and in Peters et al, Pharmacokinetic analysis: A Practical Approach (1996).

“Clearance (CL)” refers to the volume of plasma irreversibly cleared of a protein per unit time. Clearance is calculated as the Dose/AUC (AUC: is the Area Under Curve or Area under the plasma drug concentration time curve). Clearance can also be calculated by the rate of drug elimination divided by the plasma concentration of the drug (rate of elimination=CL*concentration)

“Mean Residence Time (MRT)” is the average time that the antigen binding polypeptides reside in the body before being irreversibly eliminated. Calculated as MRT=AUMC/AUC.

“Steady state concentration” (Css) is the concentration reached when the drug elimination rate becomes equal to drug administration rate as a result of continued drug administration. Css fluctuates between peak and trough levels and is measured in microgram/ml.

“Baseline” as used herein is the value of a parameter before or at the time of administration of the pharmaceutical composition of the present invention, including, for example, the value of a given biomarker or a subject's status prior to the first administration of an antibody of the present disclosure.

“Amyloid negative” means the subject does not possess brain amyloid beta plaque that is observable using positron emission tomography (“PET”) and includes, but is not limited to, subjects having a centiloid value of zero.

“Amyloid positive” means the subject possesses brain amyloid beta plaque that is observable using PET.

“ARIA risk” or “risk of ARIA” as used herein refers to the probability that a subject will develop an Amyloid Related Imaging Abnormality that is observable on by MRI. A total ARIA risk includes the risk of developing observable ARIA-E and/or observable ARIA-H. In contrast, “risk of ARIA-E” refers only to the probability that a subject will develop ARIA-E observable by MRI, irrespective of whether the subject develops observable ARIA-H, and “risk of ARIA-H” refers only to the probability that a subject will develop ARIA-E observable by MRI, irrespective of whether the subject develops observable ARIA-E. ARIA risk may be assessed at baseline (prior to the administration of an anti-amyloid β antibody of the present disclosure) or, alternatively, during or after treatment.

### Treatment Regimes

Prophylactic applications: pharmaceutical compositions or medicaments are administered to a patient susceptible to, or otherwise at risk of, Alzheimer's disease or other amyloidogenic disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Patient susceptibility or risk for developing an amyloidogenic disease can be determined, for example, from a genetic marker, a biochemical marker, unspecified hereditary risk or other means. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease.

In some embodiments, administration of agent reduces or eliminates cognitive impairment in patients that have not yet developed characteristic Alzheimer's, or other amyloidogenic disease cognitive pathology. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved, where “immune response” or “immunological response” includes the development of a humoral (antibody mediated) and/or a cellular (mediated by antigen-specific T cells or their secretion products) response directed against an antigen in a recipient subject. Such a response can be an active response, i.e., induced by administration of immunogen, or a passive response, i.e., induced by administration of immunoglobulin or antibody or primed T-cells.

In some embodiments, antibody is administered on multiple occasions. Intervals between single dosages can be weekly, monthly or yearly. In some embodiments, a single dosage may be administered about once or twice every week, about once or twice every two weeks, about once or twice every three weeks, about once or twice every four weeks, about once or twice every five weeks, or about once or twice every six weeks. In one particular embodiment, antibody is administered once about every four weeks subcutaneously.

Intervals can also be irregular as indicated by measuring blood levels of antibody to Aβ in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies.

In some embodiments, a patient with Alzheimer's disease administered a pharmaceutically effective amount of an anti-Aβ antibody (or antigen-binding fragment thereof) as described herein is treated by reduction in amyloid plaque burden, measured by PET imaging.

In another embodiment, antibody can be administered in a fixed amount at each administration. For example, a fixed amount of about 65 mg, or about 70 mg, or about 75 mg, or about 195 mg, or about 200 mg, can be administered at one time to an individual. In one particular embodiment, about 70 mg of antibody is administered once about every four weeks subcutaneously. In one particular embodiment, about 200 mg of antibody is administered once about every four weeks subcutaneously.

In another particular embodiment, about 45 mg of h2731 is administered once about every four weeks subcutaneously to an individual. In another particular embodiment, about 70 mg of h2731 is administered once about every four weeks subcutaneously to an individual. In another particular embodiment, about 200 mg of h2731 is administered once about every four weeks subcutaneously to an individual.

In another particular embodiment, about 45 mg of h2726 is administered once about every four weeks subcutaneously to an individual. In another particular embodiment, about 70 mg of h2726 is administered once about every four weeks subcutaneously to an individual. In another particular embodiment, about 200 mg of h2726 is administered once about every four weeks subcutaneously to an individual.

In another particular embodiment, about 45 mg of h2726 is administered once about every four weeks subcutaneously to an individual. In another particular embodiment, about 70 mg of h2831 is administered once about every four weeks subcutaneously to an individual. In another particular embodiment, about 200 mg of h2831 is administered once about every four weeks subcutaneously to an individual.

In another particular embodiment, about 45 mg of h2726 is administered once about every four weeks subcutaneously to an individual. In another particular embodiment, about 70 mg of h2931 is administered once about every four weeks subcutaneously to an individual. In another particular embodiment, about 200 mg of h2931 is administered once about every four weeks subcutaneously to an individual.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity. A relatively low dosage is administered at relatively infrequent intervals over a long period of time. Some patients continue to receive treatment for the rest of their lives.

In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Administration: therapeutic agents can be administered by parenteral, topical, intravenous, oral, subcutaneous, intraarterial, intracranial, intraperitoneal, intranasal, intraocular or intramuscular means for prophylactic and/or therapeutic treatment. Intramuscular injection is most typically performed in the arm or leg muscles. In some methods, agents are injected directly into a particular tissue where deposits have accumulated, for example intracranial injection. Intramuscular injection or intravenous infusion are preferred for administration of antibody. In some methods, particular therapeutic antibodies are injected directly into the cranium. In some methods, antibodies are administered as a sustained release composition or device. In some embodiments, antibodies are administered subcutaneously using an autoinjector device.

### Dosing Regimens

The present disclosure provides methods of treating neurological disorders (e.g., Alzheimer's Disease), the method comprising administering a composition comprising an anti-amyloid β antibody. For example, in some embodiments, the present disclosure provides a method of treating Alzheimer's disease in a subject, comprising administering to the subject about 20 mg to about 200 mg of an anti-amyloid β antibody or an antigen-binding fragment thereof once about every 3-5 weeks. In some embodiments, the present disclosure provides a method of treating Alzheimer's disease in a subject, comprising administering to the subject about 20 mg to about 200 mg of an anti-amyloid β antibody or an antigen-binding fragment thereof once about every 4 weeks. In example embodiments, administration is subcutaneous.

The present disclosure also provides methods of reducing amyloid plaque in a subject. For example, in some embodiments, the present disclosure provides a method of reducing amyloid plaque in a subject, comprising administering to the subject about 20 mg to about 200 mg of an anti-amyloid β antibody or an antigen-binding fragment thereof once about every 3-5 weeks. In some embodiments, the present disclosure provides a method of reducing amyloid plaque in a subject, comprising administering to the subject about 20 mg to about 200 mg of an anti-amyloid β antibody or an antigen-binding fragment thereof once about every 4 weeks. In example embodiments, administration is subcutaneous.

The present disclosure further provides methods of converting a subject from amyloid positive to amyloid negative. For example, in some embodiments, the present disclosure provides a method of converting a subject from amyloid positive to amyloid negative, comprising administering to the subject about 20 mg to about 200 mg of an anti-amyloid β antibody or an antigen-binding fragment thereof once about every 3-5 weeks. In some embodiments, the present disclosure provides a method of converting a subject from amyloid positive to amyloid negative, comprising administering to the subject about 20 mg to about 200 mg of an anti-amyloid β antibody or an antigen-binding fragment thereof once about every 4 weeks. In example embodiments, administration is subcutaneous.

In example embodiments, the method comprises administering to the subject about 45 mg of the anti-amyloid β antibody or an antigen-binding fragment thereof once about every 4 weeks. In example embodiments, the method comprises administering to the subject about 70 mg of the anti-amyloid β antibody or an antigen-binding fragment thereof once about every 4 weeks. In example embodiments, the method comprises administering to the subject about 200 mg of the anti-amyloid β antibody or an antigen-binding fragment thereof once about every 4 weeks. In example embodiments, administration is subcutaneous.

In some embodiments, the method comprises administering to the subject a pharmaceutically effective amount of the anti-amyloid β antibody or an antigen-binding fragment thereof. For example, in some embodiments, the method comprises administering to the subject up to about 200 mg (e.g., up to about 180 mg, up to about 160 mg, up to about 140 mg, up to about 120 mg, up to about 100 mg, up to about 70 mg, or up to about 50 mg). In some embodiments, the method comprises administering to the subject about 20 mg to about 200 mg (e.g., about 30 mg to about 200 mg, about 40 mg to about 200 mg, about 50 mg to about 200 mg, about 60 mg to about 200 mg, about 70 mg to about 200 mg, about 80 mg to about 200 mg, about 90 mg to about 200 mg, about 100 mg to about 200 mg, about 120 mg to about 200 mg, about 140 mg to about 200 mg, about 160 mg to about 200 mg, about 180 mg to about 200 mg, or about 190 mg to about 200 mg) of the anti-amyloid β antibody or an antigen-binding fragment thereof. For example, in some embodiments, the method comprises administering to the subject about 160 mg to about 200 mg (e.g., about 170 mg to about 200 mg, about 180 mg to about 200 mg, or about 190 mg to about 200 mg) of the anti-amyloid β antibody or an antigen-binding fragment thereof.

In some embodiments, the method comprises administering to the subject about 20 mg to about 140 mg (e.g., about 30 mg to about 140 mg, about 40 mg to about 140 mg, about 50 mg to about 140 mg, about 60 mg to about 140 mg, about 70 mg to about 140 mg, about 80 mg to about 140 mg, about 90 mg to about 140 mg, about 100 mg to about 140 mg, about 110 mg to about 140 mg, about 120 mg to about 140 mg, or about 130 mg to about 140 mg) of the anti-amyloid β antibody or an antigen-binding fragment thereof. In some embodiments, the method comprises administering to the subject about 20 mg to about 120 mg (e.g., about 30 mg to about 120 mg, about 40 mg to about 120 mg, about 50 mg to about 120 mg, about 60 mg to about 120 mg, about 70 mg to about 120 mg, about 80 mg to about 120 mg, about 90 mg to about 120 mg, about 100 mg to about 120 mg, or about 110 mg to about 120 mg) of the anti-amyloid β antibody or an antigen-binding fragment thereof. In some embodiments, the method comprises administering to the subject about 20 mg to about 100 mg (e.g., about 30 mg to about 100 mg, about 40 mg to about 100 mg, about 50 mg to about 100 mg, about 60 mg to about 100 mg, about 70 mg to about 100 mg, about 80 mg to about 100 mg, or about 90 mg to about 100 mg) of the anti-amyloid β antibody or an antigen-binding fragment thereof. In some embodiments, the method comprises administering to the subject about 20 mg to about 80 mg (e.g., about 30 mg to about 80 mg, about 40 mg to about 80 mg, about 50 mg to about 80 mg, about 60 mg to about 80 mg, or about 70 mg to about 80 mg) of the anti-amyloid β antibody or an antigen-binding fragment thereof. In some embodiments, the method comprises administering to the subject about 20 mg to about 60 mg (e.g., about 30 mg to about 60 mg, about 40 mg to about 60 mg, or about 50 mg to about 60 mg) of the anti-amyloid β antibody or an antigen-binding fragment thereof.

In example embodiments, the method comprises administering to the subject about 40 mg to about 50 mg of the anti-amyloid β antibody or an antigen-binding fragment thereof. In example embodiments, the method comprises administering to the subject about 65 mg to about 75 mg of the anti-amyloid β antibody or an antigen-binding fragment thereof. In example embodiments, the method comprises administering to the subject about 195 mg to about 205 mg of the anti-amyloid β antibody or an antigen-binding fragment thereof.

In some embodiments, the method comprises administering to the subject about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, or about 200 mg of the anti-amyloid β antibody or an antigen-binding fragment thereof. In some embodiments, the method comprises administering to the subject about 45 mg, about 70 mg, or about 200 mg of the anti-amyloid β antibody or an antigen-binding fragment thereof.

In example embodiments, the method comprises administering to the subject about 45 mg of the anti-amyloid β antibody or an antigen-binding fragment thereof. In example embodiments, the method comprises administering to the subject about 70 mg of the anti-amyloid β antibody or an antigen-binding fragment thereof. In example embodiments, the method comprises administering to the subject about 200 mg of the anti-amyloid β antibody or an antigen-binding fragment thereof.

In some embodiments, the method comprises administering the anti-amyloid β antibody or an antigen-binding fragment thereof once about every 4 weeks. In some embodiments, the method comprises administering the anti-amyloid β antibody or an antigen-binding fragment thereof once about a month. In some embodiments, the method comprises administering the anti-amyloid β antibody or an antigen-binding fragment thereof once about every 28 days.

In some embodiments, the method comprises administering the anti-amyloid β antibody or an antigen-binding fragment thereof as a single administration (e.g., a single subcutaneous injection) once about every 3-5 weeks. In some embodiments, the method comprises administering the anti-amyloid β antibody or an antigen-binding fragment thereof as a single administration once about every 4 weeks. In some embodiments, the method comprises administering the anti-amyloid β antibody or an antigen-binding fragment thereof as a single administration once every 4 weeks. In some embodiments, the method comprises administering the anti-amyloid β antibody or an antigen-binding fragment thereof as a single administration once about a month. In some embodiments, the method comprises administering the anti-amyloid β antibody or an antigen-binding fragment thereof as a single administration once about every 28 days.

In some embodiments, the method comprises administering the anti-amyloid β antibody or an antigen-binding fragment thereof as an injection. In some embodiments, the method comprises administering the anti-amyloid β antibody or an antigen-binding fragment thereof as a parenteral injection. In some embodiments, the method comprises administering the anti-amyloid β antibody or an antigen-binding fragment thereof via intravenous injection or subcutaneous injection. In example embodiments, the method comprises administering the anti-amyloid β antibody or an antigen-binding fragment thereof as a subcutaneous injection. In some embodiments, the method comprises administering the anti-amyloid β antibody or an antigen-binding fragment thereof via a syringe. In some embodiments, the method comprises administering the anti-amyloid β antibody or an antigen-binding fragment thereof via an autoinjector.

### Anti-Aβ Antibodies

The anti-amyloid β antibody or fragment includes the heavy chain CDRs and the light chain CDRs from one of the constructs identified herein as h2726, h2731, h2831, h2931, h2926, h4921, h2828, h2929, h3818G, h2927, h49k3G, h4917G h2727, and h4918G. Particular monoclonal antibodies of the disclosure may bind to an epitope within residues 1-6 of Aβ (with the first N terminal residue of natural Aβ designated 1). Some monoclonal antibodies bind to an epitope within amino acids 1-6, some to an epitope within 1-5, and some to an epitope within 1-4. Some antibodies bind to epitopes within amino acids 1-3, 2-5, 3-5, 2-4, 2-5, 2-6, 3-5, or 3-6. When an antibody is said to bind to an epitope within specified residues, such as Aβ 1-6 for example, what is meant is that the antibody specifically binds to a polypeptide containing at least one of the specified residues (i.e., at least one amino acid selected from Aβ amino acids 1-6 in this example); such antibody does not necessarily contact every residue within Aβ 1-6. In some embodiments, the antibody binds to an epitope including at least one amino acid from acid selected from amino acids 1-10 of the Aβ peptide. In another aspect, the antibody binds to an epitope including at least one amino acid from acid selected from amino acids 1-7 of the Aβ peptide. Additional amino acids of the epitope may be outside the region of amino acids 1-10 or amino acids 1-7.

In another aspect, the anti-amyloid β antibody or fragment includes a heavy chain variable region having a heavy chain CDR1, CDR2 and CDR3 and a light chain variable region comprising a light chain CDR1, CDR2 and CDR3 from the constructs show in Table 1A.

TABLE 1A

| Construct | | | SEQ | | | SEQ |
|---|---|---|---|---|---|---|
| ID | VH/VL Sequences | | ID | CDR Sequences | | ID |
| h2726 | VH | EVQLLESGGGLVQPGGS | 3 | 1 | GFTFS NYGMS | 16 |
| | | LRLSCAASGFTFSNYGM | | 2 | SIRSG SGRTY YSDNV KG | 20 |
| | | SWVRQAPGKGLEWVASI | | 3 | YDHYS GSSDY | 18 |
| | | RSGSGRTYYSDNVKGRF | | | | |
| | | TISRDNSKNTLYLQMNS | | | | |
| | | LRAEDTAVYYCVRYDHY | | | | |
| | | SGSSDYWGQGTLVTVSS | | | | |
| | VL | DVVMTQSPLSLPVTPGE | 8 | 1 | KSSQS LLDYD GKTYL N | 29 |
| | | PASISCKSSQSLLDYDG | | 2 | KVSNR DS | 33 |
| | | KTYLNWLLQKPGQSPQR | | 3 | WQGTH FPRT | 28 |
| | | LIYKVSNRDSGVPDRFS | | | | |
| | | GSGSGTDFTLKISRVEA | | | | |
| | | EDVGVYYCWQGTHFPRT | | | | |
| | | FGQGTKVEIK | | | | |
| h2731 | VH | EVQLLESGGGLVQPGGS | 3 | 1 | GFTFS NYGMS | 16 |
| | | LRLSCAASGFTFSNYGM | | 2 | SIRSG SGRTY YSDNV KG | 20 |
| | | SWVRQAPGKGLEWVASI | | 3 | YDHYS GSSDY | 18 |
| | | RSGSGRTYYSDNVKGRF | | | | |
| | | TISRDNSKNTLYLQMNS | | | | |
| | | LRAEDTAVYYCVRYDHY | | | | |
| | | SGSSDYWGQGTLVTVSS | | | | |
| | VL | DVVMTQSPLSLPVTLGE | 9 | 1 | KSSQS LLDYD GKTYL N | 29 |
| | | PASISCKSSQSLLDYDG | | 2 | RVTNR DT | 34 |
| | | KTYLNWLLQKPGQSPQR | | 3 | WQGTH FPRS | 38 |
| | | LIYRVTNRDTGVPDRFS | | | | |
| | | GSGSGTDFTLKISRVEA | | | | |
| | | EDVGVYYCWQGTHEPRS | | | | |
| | | FGQGTKVEIK | | | | |
| h2831 | VH | EVQLLESGGGLVQPGGS | 4 | 1 | GFTFS NFGMS | 19 |
| | | LRLSCAASGFTFSNFGM | | 2 | SVRSG SGRTY YSDNV KG | 21 |
| | | SWVRQAPGKGLEWVASV | | 3 | YDHYS GTSDY | 24 |
| | | RSGSGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTLGE | 9 | 1 | KSSQS LLDYD GKTYL N | 29 |
| | | PASISCKSSQSLLDYDG | | 2 | RVTNR DT | 34 |
| | | KTYLNWLLQKPGQSPQR | | 3 | WQGTH FPRS | 38 |
| | | LIYRVTNRDTGVPDRFS | | | | |
| | | GSGSGTDFTLKISRVEA | | | | |
| | | EDVGVYYCWQGTHEPRS | | | | |
| | | FGQGTKVEIK | | | | |
| h2931 | VH | EVQLLESGGGLVQPGGS | 5 | 1 | GFTFS NFGMS | 19 |
| | | LRLSCAASGFTFSNFGM | | 2 | SVRSG SGRTY YSDNV KG | 21 |
| | | SWVRQAPGKGLEWVASV | | 3 | YDHYT GTSDY | 25 |
| | | RSGSGRTYYSDNVKGRF | | | | |
| | | TISRDNSKNTLYLQMNS | | | | |
| | | LRAEDTAVYYCVRYDHY | | | | |
| | | TGTSDYWGQGTLVTVSS | | | | |
| | VL | DVVMTQSPLSLPVTLGE | 9 | 1 | KSSQS LLDYD GKTYL N | 29 |
| | | PASISCKSSQSLLDYDG | | 2 | RVTNR DT | 34 |
| | | KTYLNWLLQKPGQSPQR | | 3 | WQGTH FPRS | 38 |
| | | LIYRVTNRDTGVPDRFS | | | | |
| | | GSGSGTDFTLKISRVEA | | | | |
| | | EDVGVYYCWQGTHEPRS | | | | |
| | | FGQGTKVEIK | | | | |
| h2926 | VH | EVQLLESGGGLVQPGGS | 5 | 1 | GFTFS NFGMS | 19 |
| | | LRLSCAASGFTFSNFGM | | 2 | SVRSG SGRTY YSDNV KG | 21 |
| | | SWVRQAPGKGLEWVASV | | 3 | YDHYT GTSDY | 25 |
| | | RSGSGRTYYSDNVKGRF | | | | |
| | | TISRDNSKNTLYLQMNS | | | | |
| | | LRAEDTAVYYCVRYDHY | | | | |
| | | TGTSDYWGQGTLVTVSS | | | | |
| | VL | DVVMTQSPLSLPVTPGE | 8 | 1 | KSSQS LLDYD GKTYL N | 29 |
| | | PASISCKSSQSLLDYDG | | 2 | KVSNR DS | 33 |
| | | KTYLNWLLQKPGQSPQR | | 3 | WQGTH FPRT | 28 |
| | | LIYKVSNRDSGVPDRES | | | | |
| | | GSGSGTDFTLKISRVEA | | | | |
| | | EDVGVYYCWQGTHFPRT | | | | |
| | | FGQGTKVEIK | | | | |
| h4921G | VH | EVQLLESGGGLVQPGGS | 6 | 1 | GFTFS NFGMS | 19 |
| | | LRLSCAASGFTFSNFGM | | 2 | SVRSG GGRTY YSDNV KG | 22 |
| | | SWVRQAPGKGLEWVASV | | 3 | YDHYS GTSDY | 24 |
| | | RSGGGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTLGE | 10 | 1 | KSSQS LLDSD GKTYL N | 26 |
| | | PASISCKSSQSLLDSDG | | 2 | RVTNR DT | 34 |
| | | KTYLNWLLQKPGQSPQR | | 3 | WQGTH FPRT | 28 |
| | | LIYRVTNRDTGVPDRFS | | | | |
| | | GSGSGTDFTLKISRVEA | | | | |
| | | EDVGVYYCWQGTHEPRT | | | | |
| | | FGQGTKVEIK | | | | |
| h2826 | VH | EVQLLESGGGLVQPGGS | 4 | 1 | GFTFS NFGMS | 19 |
| | | LRLSCAASGFTFSNFGM | | 2 | SVRSG SGRTY YSDNV KG | 21 |
| | | SWVRQAPGKGLEWVASV | | 3 | YDHYS GTSDY | 24 |
| | | RSGSGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTPGE | 8 | 1 | KSSQS LLDYD GKTYL N | 29 |
| | | PASISCKSSQSLLDYDG | | 2 | KVSNR DS | 33 |
| | | KTYLNWLLQKPGQSPQR | | 3 | WQGTH FPRT | 28 |
| | | LIYKVSNRDSGVPDRES | | | | |
| | | GSGSGTDFTLKISRVEA | | | | |
| | | EDVGVYYCWQGTHEPRT | | | | |
| | | FGQGTKVEIK | | | | |
| h2929 | VH | EVQLLESGGGLVQPGGS | 5 | 1 | GFTFS NFGMS | 19 |
| | | LRLSCAASGFTFSNFGM | | 2 | SVRSG SGRTY YSDNV KG | 21 |
| | | SWVRQAPGKGLEWVASV | | 3 | YDHYT GTSDY | 25 |
| | | RSGSGRTYYSDNVKGRF | | | | |
| | | TISRDNSKNTLYLQMNS | | | | |
| | | LRAEDTAVYYCVRYDHY | | | | |
| | | TGTSDYWGQGTLVTVSS | | | | |
| | VL | DVVMTQSPLSLPVTPGE | 11 | 1 | RSSQS LVDYD GKTYL N | 31 |
| | | PASISCRSSQSLVDYDG | | 2 | KVSNR DS | 33 |
| | | KTYLNWLLQRPGQSPQR | | 3 | WQGSH FPRS | 39 |
| | | LIYKVSNRDSGVPDRES | | | | |
| | | GSGSGTDFTLKISRVEA | | | | |
| | | EDVGVYYCWQGSHEPRS | | | | |
| | | YGQGTKVEIK | | | | |
| h3818G | VH | EVQLLESGGGLVQPGGS | 7 | 1 | GFTFA NYGMS | 20 |
| | | LRLSCAASGFTFANYGM | | 2 | SVRSG GSRTY YSDNV KG | 23 |
| | | SWVRQAPGKGLEWVASV | | 3 | YDHYS GSSDY | 18 |
| | | RSGGSRTYYSDNVKGRE | | | | |
| | | TISRDNSKNTLYLQMNS | | | | |
| | | LRAEDTAVYYCVRYDHY | | | | |
| | | SGSSDYWGQGTLVTVSS | | | | |
| | VL | DVVMTQSPLSLPVTLGE | 12 | 1 | KSSQS LMDTD GKTYL N | 32 |
| | | PASISCKSSQSLMDTDG | | 2 | KVSNR ES | 35 |
| | | KTYLNWLLQKPGQSPQR | | 3 | WQGTH FPRT | 28 |
| | | LIYKVSNRESGVPDRES | | | | |
| | | GSGSGTDFTLKISRVEA | | | | |
| | | EDVGVYYCWQGTHEPRT | | | | |
| | | FGQGTKVEIK | | | | |
| h2927 | VH | EVQLLESGGGLVQPGGS | 5 | 1 | GFTES NFGMS | 19 |
| | | LRLSCAASGFTFSNFGM | | 2 | SVRSG SGRTY YSDNV KG | 21 |
| | | SWVRQAPGKGLEWVASV | | 3 | YDHYT GTSDY | 25 |
| | | RSGSGRTYYSDNVKGRF | | | | |
| | | TISRDNSKNTLYLQMNS | | | | |
| | | LRAEDTAVYYCVRYDHY | | | | |
| | | TGTSDYWGQGTLVTVSS | | | | |
| | VL | DVVMTQSPLSLPVTPGE | 13 | 1 | KSSQS LLDYD GKTYL N | 29 |
| | | PASISCKSSQSLLDYDG | | 2 | KVSNR DS | 33 |
| | | KTYLNWLLQKPGQSPQR | | 3 | WQGTH FPRS | 38 |
| | | LIYKVSNRDSGVPDRFS | | | | |
| | | GSGSGTDFTLKISRVEA | | | | |
| | | EDVGVYYCWQGTHFPRS | | | | |
| | | FGQGTKVEIK | | | | |
| h49k3G | VH | EVQLLESGGGLVQPGGS | 6 | 1 | GFTFS NFGMS | 19 |
| | | LRLSCAASGFTFSNFGM | | 2 | SVRSG GGRTY YSDNV KG | 22 |
| | | SWVRQAPGKGLEWVASV | | 3 | YDHYS GTSDY | 24 |
| | | RSGGGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTLGE | 14 | 1 | KSSQS LLDSD GKTYL N | 26 |
| | | PASISCKSSQSLLDSDG | | 2 | KVSNR DS | 33 |
| | | KTYLNWLLQKPGQSPQR | | 3 | WQGTH FPRT | 28 |
| | | LIYKVSNRDSGVPDRFS | | | | |
| | | GSGSGTDFTLKISRVEA | | | | |
| | | EDVGVYYCWQGTHFPRT | | | | |
| | | FGQGTKVEIK | | | | |
| h4917G | VH | EVQLLESGGGLVQPGGS | 6 | 1 | GFTFS NFGMS | 19 |
| | | LRLSCAASGFTFSNFGM | | 2 | SVRSG GGRTY YSDNV KG | 22 |
| | | SWVRQAPGKGLEWVASV | | 3 | YDHYS GTSDY | 24 |
| | | RSGGGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTLGE | 15 | 1 | KSSQS LLDSD GKTYL N | 26 |
| | | PASISCKSSQSLLDSDG | | 2 | KVTNR ES | 36 |
| | | KTYLNWLLQKPGQSPQR | | 3 | WQGTH FPRS | 38 |
| | | LIYKVTNRESGVPDRES | | | | |
| | | GSGSGTDFTLKISRVEA | | | | |
| | | EDVGVYYCWQGTHEPRS | | | | |
| | | FGQGTKVEIK | | | | |
| h2727 | VH | EVQLLESGGGLVQPGGS | 3 | 1 | GFTFS NYGMS | 16 |
| | | LRLSCAASGFTFSNYGM | | 2 | SIRSG SGRTY YSDNV KG | 20 |
| | | SWVRQAPGKGLEWVASI | | 3 | YDHYS GSSDY | 18 |
| | | RSGSGRTYYSDNVKGRF | | | | |
| | | TISRDNSKNTLYLQMNS | | | | |
| | | LRAEDTAVYYCVRYDHY | | | | |
| | | SGSSDYWGQGTLVTVSS | | | | |
| | VL | DVVMTQSPLSLPVTPGE | 13 | 1 | KSSQS LLDYD GKTYL N | 29 |
| | | PASISCKSSQSLLDYDG | | 2 | KVSNR DS | 33 |
| | | KTYLNWLLQKPGQSPQR | | 3 | WQGTH FPRS | 38 |
| | | LIYKVSNRDSGVPDRFS | | | | |
| | | GSGSGTDFTLKISRVEA | | | | |
| | | EDVGVYYCWQGTHEPRS | | | | |
| | | FGQGTKVEIK | | | | |
| h4918G | VH | EVQLLESGGGLVQPGGS | 6 | 1 | GFTES NFGMS | 19 |
| | | LRLSCAASGFTFSNFGM | | 2 | SVRSG GGRTY YSDNV KG | 22 |
| | | SWVRQAPGKGLEWVASV | | 3 | YDHYS GTSDY | 24 |
| | | RSGGGRTYYSDNVKGRF | | | | |
| | | TISRDNSKNTLYLQMNS | | | | |
| | | LRAEDTAVYYCVRYDHY | | | | |
| | | SGTSDYWGQGTLVTVSS | | | | |
| | VL | DVVMTQSPLSLPVTLGE | 12 | 1 | KSSQS LMDTD GKTYL N | 32 |
| | | PASISCKSSQSLMDTDG | | 2 | KVSNR ES | 25 |
| | | KTYLNWLLQKPGQSPQR | | 3 | WQGTH FPRT | 28 |
| | | LIYKVSNRESGVPDRFS | | | | |
| | | GSGSGTDFTLKISRVEA | | | | |
| | | EDVGVYYCWQGTHFPRT | | | | |
| | | FGQGTKVEIK | | | | |

In another aspect the anti-amyloid β antibody or fragment of the disclosure includes a heavy chain variable region (VH) as shown for one of the constructs in Table 1. The anti-amyloid β antibody or fragment may also include light chain variable region (VL) as shown for one of the constructs in Table 1A.

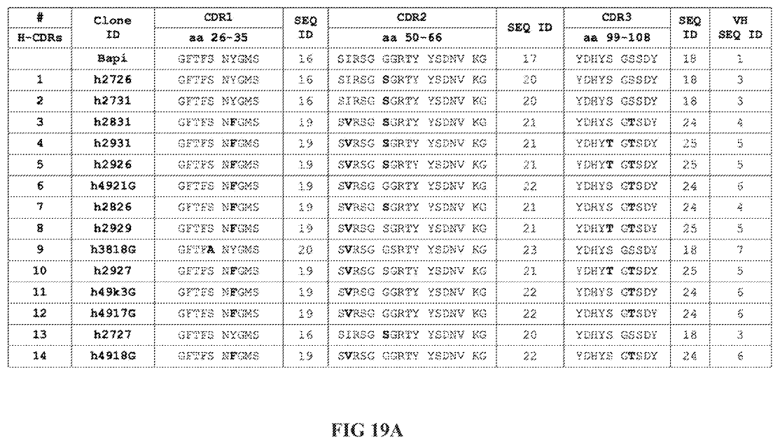

An alignment of the CDRs for each of the heavy chain and light chain sequences identified in Table 1A and the CDRs from bapineuzumab (“Bapi”, “hBP”) is show in FIGS. 19A and 19B. In one aspect, the disclosure is directed an antibody or fragment thereof including a heavy chain CDR1, CDR2, and CDR3, wherein CDR1 may be selected from any one of SEQ ID NOS: 16, 19 and 20, wherein CDR2 may be selected from any one of SEQ ID NOS: 17. 20, 21 22, and 23 and wherein CDR3 may be selected from any one of SEQ ID NOS: 18, 24, 25. In addition, the anti-amyloid β antibody or fragment thereof includes a light chain CDR1, CDR2, and CDR3, wherein CDR1 may be selected from any one of SEQ ID NOS: 26, 29, 31, and 32, wherein CDR2 may be selected from any one of SEQ ID NOS: 27, 33, 34 and 35, and wherein CDR3 may be selected from any one of SEQ ID NOS: 28, 38 and 39. In each of these embodiments, the heavy chain CDRs and the light chain CDRs are not, in combination, simultaneously SEQ ID NOS: 16, 17, 18, 26, 27 and 28.

Analysis of protein modeling information for the antibodies described above identified two changes in the CDRs that, among others, were the contributors to increased avidity/affinity characteristics of the antibodies of the disclosure:
CDR-L1: S32Y (Ser to Tyr at position 32), and
CDR-H2: G55S (Gly to Ser at position 55)

Anti-Aβ antibodies with Tyr at position 32 in CDR-L1 and Ser at position 55 in CDR-H2 that bind the same epitope bound by antibodies listed herein are expected to have the same properties as the listed identified antibodies (See Table 1A and FIG. 19A and FIG. 19B). Antibodies disclosed that do not have Tyr at position 32 in CDR-L1 and Ser at position 55 in CDR-H2 can be modified to possess Tyr at position 32 in CDR-L1 and Ser at position 55 in CDR-H2 and can be expected to confer similar binding properties to such antibodies identified herein.

Examples of a CDR-L1 with Tyr at position 32 include SEQ NOs: 29 and 31. Examples of a CDR-H2 with Ser at position 55 include SEQ Nos: 20 and 21.

As examples, antibodies comprising a CDR-L1 with Tyr at position 32 and a CDR-H2 with Ser at position 55 include antibodies with the CDRs of h2726, h2731, h2727, h2826, h2831, h2926, h2927, h2931, h2929 (See Table 1A). Additional such antibodies include antibodies comprising LC CDRs 1, 2, 3 and HC CDRs 1, 2, 3 as set forth in the table below in Table 1B.

TABLE 1B

| | | CDR Sequences | | SEQ |
|---|---|---|---|---|
| Antibody | HC/LC | (HC 1, 2, 3; LC 1, 2, 3) | | ID |
| h2729 | HC | 1 | GFTFS NYGMS | 16 |
| | | 2 | SIRSG SGRTY YSDNV KG | 20 |
| | | 3 | YDHYS GSSDY | 18 |
| | LC | 1 | RSSQS LVDYD GKTYL N | 31 |
| | | 2 | KVSNR DS | 33 |
| | | 3 | WQGSH FPRS | 39 |
| h2829 | HC | 1 | GFTFS NFGMS | 19 |
| | | 2 | SVRSG SGRTY YSDNV KG | 21 |
| | | 3 | YDHYS GTSDY | 24 |
| | LC | 1 | RSSQS LVDYD GKTYL N | 31 |
| | | 2 | KVSNR DS | 33 |
| | | 3 | WQGSH FPRS | 39 |
| h2827 | HC | 1 | GFTFS NFGMS | 19 |
| | | 2 | SVRSG SGRTY YSDNV KG | 21 |
| | | 3 | YDHYS GTSDY | 24 |
| | LC | 1 | KSSQS LLDYD GKTYL N | 29 |
| | | 2 | RVTNR DT | 33 |
| | | 3 | WQGTH FPRS | 38 |
| HC-S55/ | HC | 1 | GFTFS NYGMS | 16 |
| LC-Y32 | | 2 | SIRSG SGRTY YSDNV KG | 20 |
| | | 3 | YDHYS GSSDY | 18 |
| | LC | 1 | KSSQS LLDYD GKTYL N | 29 |
| | | 2 | LVSKL DS | 27 |
| | | 3 | WQGTH FPRT | 28 |

In view of the binding properties identified for the antibodies identified herein, consensus sequences can be identified that would be expected to provide similar binding properties. For example, in embodiments of the disclosure, antibodies or binding fragments thereof that that specifically bind to Aβ peptide may include heavy chain variable regions having heavy chain CDR1, CDR2 and CDR3 and a light chain variable regions having light chain CDR1, CDR2 and CDR3, as follows:
heavy chain CDR1 comprises amino acid sequence GFTFSNX₁GMS, wherein X₁ is Y or F (SEQ ID NO: 88);
heavy chain CDR2 comprises amino acid sequence SX₁RSGSGRTYYSDNVKG, wherein is X₁ is I or V (SEQ ID NO: 89);
heavy chain CDR3 comprises amino acid sequence YDHYX₁GX_{b 2}SDY, wherein X₁ is S or T and X₂ is S or T (SEQ ID NO: 90);
light chain CDR1 comprises amino acid sequence KSSQSLLDYDGKTYLN (SEQ ID NO: 91);
light chain CDR2 comprises amino acid sequence X₁VX₂NRDX₃, wherein X₁ is K or R, X₂ is S or T, and X₃ is S or T (SEQ ID NO: 92).

light chain CDR3 comprises amino acid sequence WQGTHFPRX₁, wherein X₁ is S or T (SEQ ID NO: 93).

In some embodiments, the light chain CDR3 comprises WQGTHFPRXIFX2, wherein X₁ is S or T and X₂ is F or Y (SEQ ID NO: 94).

Similar consensus sequences that may be expected to provide binding properties similar to the antibodies described herein include a heavy chain variable region having heavy chain CDR1, CDR2 and CDR3 and a light chain variable region having light chain CDR1, CDR2 and CDR3, as follows:
heavy chain CDR1 comprises amino acid sequence GFTFXINX₂GMS, wherein X₁ is S or A, and X₂ is Y or F (SEQ ID NO: 95);
heavy chain CDR2 comprises amino acid sequence SX₁RSGX₂X₃RTYYSDNVKG, wherein is X₁ is I or V, X₂ is S or G and X₃ is S or G (SEQ ID NO: 96);
heavy chain CDR3 comprises amino acid sequence YDHYX₁GX₂SDY, wherein X₁ is S or T and X₂ is S or T (SEQ ID NO: 90);
light chain CDR1 comprises amino acid sequence X₁SSQSLX₂DX₃DGKTYLN, wherein X₁ is K or R, X₂ is V, M or L, and X₃ is Y, T or S (SEQ ID NO: 97);
light chain CDR2 comprises amino acid sequence X₁VX₂NRX₃X₄, wherein X₁ is K or R, X₂ is S or T, and X₃ is E or D, and X₄ is S or T (SEQ ID NO: 98).
light chain CDR3 comprises amino acid sequence WQGX₁HFPRX₂, wherein X₁ is S or T, and X₂ is S or T (SEQ ID NO: 99).

In some embodiments, the light chain CDR3 comprises WQGTHFPRX₁FX₂X₃, wherein X₁ is S or T, X₂ is S or T and X₃ is F or Y (SEQ ID NO: 100).

In addition, the light and heavy variable regions may be at least at least 75% identical to the light and heavy chain variable regions show in Table 1A. For example, the light and heavy chain variable regions may be 75% identical, 80%, identical, 85% identical, 90% identical, 95% identical, 96% identical, 97% identical, 98% identical, 99% identical, of 100% identical to VH and/or VL sequences identified in Table 1A. In various aspects, any sequence variation in the VH and VL may be present outside the CDRs so that the VH and VL sequences of the disclosure include the CDRs identified in Table 1A, but the regions of the VH and VL sequences outside of the CDRs (e.g., a region excluding the CDRs) may be at least 75% identical to the regions outside the CDRs of the VH and VL sequences in Table 1A.

For example, the anti-amyloid β antibody or fragment of the disclosure may include a heavy chain variable region, excluding the CDRS, that is at least 95% identical to one of SEQ ID NOS: 3, 4, 5, 6 and 7, and the light chain variable region, excluding the CDRs, that is at least 95% identical to one of SEQ ID NOS: 8, 9, 10, 11, 12, 13, 14 and 15.

The antibodies and fragments of the disclosure may also include a heavy chain constant region that is at least 75% identical to SEQ ID NO: 40. For example, the heavy chain constant region may be 75% identical, 80%, identical, 85% identical, 90% identical, 95% identical, 96% identical, 97% identical, 98% identical, 99% identical, of 100% identical to SEQ ID NO: 40.

The antibodies and fragments of the disclosure may also include a light chain constant region that is at least 75% identical to SEQ ID NO: 41. For example, the light chain constant region may be 75% identical, 80%, identical, 85% identical, 90% identical, 95% identical, 96% identical, 97% identical, 98% identical, 99% identical, of 100% identical to SEQ ID NO: 41.

A variant antibodies or fragments that are less than 100% identical to the sequences described in Table 1A (plus any constant region) can differ from an anti-Aβ antibody of Table 1A by as few as 1 to 15 amino acid residues, as few as 1 to 10 amino acid residues, such as 6-10, as few as 5, as few as 4, 3, 2, or even 1 amino acid residue. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity (e.g., the ability to bind an Aβ polypeptide).

For example, it is possible to introduce mutations only in a framework region(s) of the antibody molecules (i.e., in region(s) excluding the CDRs). Introduced mutations can be silent or neutral missense mutations, i.e., have no, or little, effect on an antibody's ability to bind antigen. These types of mutations can be useful to optimize codon usage, or improve a hybridoma's antibody production. Alternatively, non-neutral missense mutations can alter an antibody's ability to bind antigen. One of skill in the art would be able to design and test mutant molecules with desired properties such as no alteration in antigen binding activity or alteration in binding activity (e.g., improvements in antigen binding activity or change in antibody specificity). Following mutagenesis, the encoded protein can routinely be expressed and the functional and/or biological activity of the encoded protein, (e.g., ability to immunospecifically bind at least one epitope of an Aβ polypeptide) can be determined using techniques described herein or by routinely modifying techniques known in the art. The anti-amyloid β antibody h2731 possesses several physicochemical properties that make it suitable for use in methods of the present disclosure, including, for example, properties described in Tables 2 and 3. Additional properties, including pharmacokinetic parameters, are discussed herein.

TABLE 2A

| IC₅₀ Ratio | IC₅₀ | EC₅₀ | EC₅₀ |
|---|---|---|---|
| (h2731:hBP) | (μg/mL h2731) | (ng/ml h2731) | (h2731 ng/ml) |
| Aβ₁₋₄₂ fibrils | Aβ aggregates | Aβ₁₋₄₂ fibrils | Aβ_{pE3-42} fibrils |
| 0.61 | 5.024 | 36.71 | >100 |

Table 2A provides IC₅₀ and/or EC₅₀ values data for h2731 binding various amyloid β species, including Aβ₁₋₄₂ fibrils, Aβ₁₋₄₂ aggregates and Aβ_{pE3-42} fibrils. See, e.g., U.S. Pat. No. 11,440,953.

An assay based on the competition (inhibition) of binding of a labeled antibody to an antigen-coated plate was used to determine IC₅₀ for h2731. The IC₅₀ was then divided by the IC₅₀ for bapineuzumab (hBP) to yield a half maximal inhibitory concentration (IC₅₀) ratio, shown in Column 1 of Table 2A. The ratio of 0.61 demonstrates that h2731 exhibits better performance than hBP in binding Aβ₁₋₄₂ fibrils. Using a similar competition assay, it was demonstrated that h2731 exhibits an IC₅₀ value of 5.024 μg/mL (Column 2 of Table 2A), which several fold lower than the IC₅₀ value observed for hBP.

The direct binding of h2731 to Aβ₁₋₄₂ and Aβ_{pE3-42} fibrils was also assessed by ELISA (Columns 3 and 4 of Table 2A), providing an EC₅₀ value of 36.71 ng/mL for h2731 against Aß1-42 fibrils. h2731 demonstrated strong affinity to fibrils and significantly greater avidity than aducanumab. Further, a 3-fold increase in assay signal (OD490) and a 15 to 20-fold lower estimated EC₅₀ indicated increased overall binding and relative avidity of h2731 to fibrillar Aβ relative to aducanumab.

However, while h2731 binds with high apparent affinity to the N-terminus of full length Aβ, it does not specifically bind to pyroglutamate-modified Aβ (Aβ_{pE3-42}). h2731 bound with a half-maximal effective concentration (EC₅₀) of 8.1 ng/mL (54 μM) to fibrillar Aβ species with an unmodified N-terminus (Aβ₁₋₄₂). h2731 demonstrated no detectable binding to Aβ_{pE3-42} up to 100 ng/ml.

TABLE 2B

| | 1:1 binding | | Apparent | |
|---|---|---|---|---|
| | ka (1/Ms) | kd (1/s) | KD (M) | Rmax (RU) |
| Aβ₁₋₄₂ fibrils | 3.72e+5 | 2.62e−5 | 7.04e−11 | 50.7 |
| Aβ₁₋₂₈ | 1.19e+5 | 5.95e−4 | 5.01e−9 | 78.3 |

Abeta: amyloid beta, Aβ; ka: association rate constant; kd: dissociation rate constant;
KD: apparent equilibrium dissociation constant; mAb: monoclonal antibody; Rₘₐₓ: maximum response; SPR: surface plasmon resonance.

Table 2B provides additional amyloid β binding data for h2731, including data on binding dyamics of h2731 to recombinant Aβ₁₋₄₂ fibrils and Aß₁₋₂₈ monomer. As shown in Column 2 of Table 3A, h2731 binds Aβ₁₋₄₂ fibrils and Aß₁₋₂₈ monomer with association constants of 3.72×10⁵ M⁻¹ s⁻¹ and 1.19×10⁵ M⁻¹ s⁻¹ respectively. Although aducanumab binds Aβ fibrils at a faster association rate (ka), the much slower dissociation rate (kd) of h2731 of the disclosure resulted in greater measured avidity (i.e., lower KD*) than aducanumab.

Off-rate data (kd) are for h2731 shown in Column 3 of Table 3A, providing kd values of 2.62×10⁻⁵ s⁻¹ and 5.95×10⁻⁴ s⁻¹ for h2731 against Aβ₁₋₄₂ fibrils and Aß₁₋₂₈ monomer, respectively. The enhanced relative avidity of h2731 of the disclosure for fibrillar Aβ observed by ELISA was confirmed by SPR equilibrium binding kinetics (Column 4 of Table 3A), which indicated a 5- to 11-fold greater avidity (apparent KD) than aducanumab, including 4-7 nM binding affinity for Aß₁₋₂₈ monomer.

In additional embodiments, the methods of the present disclosure may utilize one or more of several different anti-amyloid β antibodies or fragments thereof. In particular, antibodies suitable for use in methods of the present disclosure possess physiochemical and pharmacological properties, discussed herein, that allow for therapeutically effective dosing using once monthly subcutaneous administration. Additional exemplary anti-amyloid β antibodies suitable for use in methods of the present disclosure include those in U.S. Pat. No. 11,440,953, which is herein incorporated by reference in its entirety.

In each of the foregoing embodiments, the anti-amyloid β antibody or fragment of the disclosure may be a humanized antibody as described herein. For example, the antibody may be a human IgG1 antibody. In addition, the antibody may a full antibody, a chimeric antibody, a CDR-grafted antibody, or a recombinant antibody. Fragments of the antibody may be a Fab, Fab′, F(ab′)2, Fabc, or Fv. Fragments are produced by recombinant DNA techniques, or by enzymatic or chemical separation of intact immunoglobulins.

The anti-amyloid β antibody or binding fragments, variant, or derivative disclosed herein can be said to bind to Aβ) or a fragment or variant thereof with an off rate (k (off)) of less than or equal to 5×10⁻² sec⁻¹, 10⁻² sec⁻¹, 5×10⁻³ sec⁻¹ or 10⁻³ sec⁻¹. In certain embodiments, an antibody of the disclosure can be said to bind Aβ or a fragment or variant thereof with an off rate (k(off)) less than or equal to 5×10⁴ sec⁻¹, 10⁻⁴ sec⁻¹, 5×10⁻⁵ sec⁻¹, or 10⁻⁵ sec⁻¹, 5×10⁻⁶ sec⁻¹, 10⁻⁶ sec⁻¹, 5×10⁻⁷ sec⁻¹ or 10⁻⁷ sec⁻¹.

An antibody or antigen-binding fragment, variant, or derivative disclosed herein can be said to bind a target polypeptide disclosed herein (e.g., Aβ) or a fragment or variant thereof with an on rate (k(on)) of greater than or equal to 10³ M⁻¹ sec⁻¹, 5×10³ M⁻¹ sec⁻¹, 10⁴ M⁻¹ sec⁻¹ or 5×10⁴ M⁻¹ sec⁻¹. In certain embodiments, an antibody of the disclosure can be said to bind a target polypeptide disclosed herein (e.g., Aβ) or a fragment or variant thereof with an on rate (k(on)) greater than or equal to 10⁵ M⁻¹ sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

Anti-Aβ antibodies or antigen-binding fragments, variants or derivatives thereof, as described herein can also be described or specified in terms of their binding affinity Aβ. Binding affinities can include those with a dissociation constant or Kd less than 5×10⁻²M, 10⁻² M, 5×10⁻³ M, 10⁻³M, 5×10⁻⁴M, 10⁻⁴M, 5×10⁻⁵M, 10⁻⁵M, 5×10⁻⁶M, 10⁻⁶M, 5×10⁻⁷M, 10⁻⁷M, 5×10⁻⁸M, 10⁻⁸ M, 5×10⁻⁹M, 10⁻⁹M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Agents of the disclosure can optionally be administered in combination with other agents that are at least partly effective in treatment of amyloidogenic disease. In the case of Alzheimer's and Down's syndrome, in which amyloid deposits occur in the brain, agents of the disclosure can also be administered in conjunction with other agents that increase passage of the agents of the disclosure across the blood-brain barrier.

### Expression of Recombinant Antibodies

The disclosure is also directed to recombinant polynucleotides encoding antibodies which, when expressed, include the heavy and light chain CDRs of the antibodies of the disclosure. Exemplary polynucleotides, which on expression code for the polypeptide chains comprising the heavy and light chain CDRs of monoclonal antibodies are provided herein (e.g., SEQ ID NO: 42 through SEQ ID NO: 69), which code for the variable light and heavy chain polypeptides, and CDRs thereof, according to SEQ ID NO: 3 through SEQ ID NO: 39. Due to codon degeneracy, other polynucleotide sequences can be readily substituted for those sequences.

Humanized and human antibodies are typically produced by recombinant expression. Nucleic acids encoding humanized light and heavy chain variable regions may be linked to constant regions are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences.

One prokaryotic host useful for cloning the polynucleotides of the present disclosure is E. coli. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, are also useful for expression. Saccharomyces is a preferred yeast host, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization. Additionally, plants (e.g., rice, tobacco) are useful for expression.

Mammalian tissue cell culture may also be used to express and produce the polypeptides of the present disclosure (e.g., polynucleotides encoding immunoglobulins or fragments thereof). Eukaryotic cells can be particularly useful because a number of suitable host cell lines capable of secreting heterologous proteins (e.g., intact immunoglobulins) have been developed in the art, and include CHO cell lines, various Cos cell lines, HeLa cells, preferably, myeloma cell lines, or transformed B-cells or hybridomas. Preferably, the cells are nonhuman. Expression vectors for these cells can include expression control sequences, such as an origin of replication, a promoter, and an enhancer, and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like.

Antibody-coding sequences can be incorporated in transgenes for introduction into the genome of a transgenic animal and subsequent expression in the milk of the transgenic animal. Suitable transgenes include coding sequences for light and/or heavy chains in operable linkage with a promoter and enhancer from a mammary gland specific gene, such as casein or beta lactoglobulin.

Vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

When heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms of the present disclosure can be purified according to standard procedures of the art, including ammonium sulfate precipitation, affinity columns (e.g., Protein A), column chromatography, HPLC purification, gel electrophoresis and the like. Substantially pure immunoglobulins of at least about 90 to 95% homogeneity are preferred, and 98 to 99% or more homogeneity most preferred, for pharmaceutical uses.

Increasing the copy number of expression vectors containing polynucleotide sequences of interest is desirable as a way to increase the production of antibodies or antibody fragments. A number of ways to genetically manipulate cells for this purpose and subsequently select the best cells are known in the art. These methods often include an “amplification” step to increase the copy number of the incorporated expression vector to improve the yield obtained for the desired protein. Amplification methods have been previously reported, e.g., by Bebbington and Hentschel (DNA Cloning Volume III (IRL press, 1987)). Any of a number of selectable markers, often in the form of nucleic acid sequences that encode enzymes that are involved in host cell metabolism and are essential for their survival under certain media conditions, can be operably linked to an expression vector, whereby the expression of a desired protein can be promoted upon selection for a selectable marker. Cells selected for a high copy number can be subjected to further amplification methods when the titer of the protein is not acceptably elevated. Such methods can involve subjecting the cells to certain toxic drugs that inhibit the selectable marker (e.g., methotrexate and dihydrofolate reductase, methionine sulphoximine and glutamine synthase, multidrug resistance/adriamycin). Through such inhibition, cell populations with increased levels of expression of this marker may be selected. This often leads to increased expression levels of similarly functionally linked expression cassettes. Vector copy number in individual cells subjected to the amplification method are assessed until a plateau of protein production is reached, preferably at least about 100 mg/ml/10⁶ cells/24 hours. Clones that grow through such selection and amplification are subsequently screened for titer/yield to select the best clone and then further evaluated. From such titration and screening, it is common to identify one or a small number of clones for subsequent production of one or more desired proteins and subsequently use it or them alone.

### Pharmaceutical Compositions

In some embodiments, the anti-Aβ antibody or fragment thereof is administered as part of a pharmaceutical composition. Several methods of preparing pharmaceutical compositions comprising anti-Aβ antibodies, or antigen-binding fragments, variants, or derivatives thereof to a subject in need thereof are known. In some embodiments, the anti-Aβ antibodies or antigen-binding fragments thereof are formulated for parenteral administration. In example embodiments, the anti-Aβ antibodies or antigen-binding fragments thereof are formulated for subcutaneous injection.

For the purposes of the disclosure, a pharmaceutically effective amount of an anti-Aβ antibody, or antigen-binding fragment, variant, or derivative thereof, means an amount sufficient to achieve effective binding to a target and to achieve a benefit, e.g., reduce brain amyloid plaques without affecting vascular amyloid, or minimizes the occurrence of microhemorrhage during chronic dosing of the anti-Aβ antibody or antigen-binding fragment thereof. In some embodiments, an anti-Aβ antibody or antigen-binding fragment, variant, or derivative thereof crosses the blood-brain barrier in an effective amount to reduce brain amyloid plaques.

The amount of an anti-Aβ antibody, or fragment, variant, or derivative thereof, to be combined with the carrier materials to produce a single dosage form will vary depending upon the subject treated and the particular mode of administration. Dosage regimens also can be adjusted to provide the optimum desired response (e.g., a therapeutic or prophylactic response).

The present disclosure provides several pharmaceutically effective amounts of anti-Aβ antibodies or antigen-binding fragment thereof (e.g., about 20 mg to about 200 mg and additional amounts and/or ranges disclosed herein). The present disclosure therefore provides the use of pharmaceutical compositions comprising these amounts in the methods disclosed herein. Such pharmaceutically effective amounts can be administered as a single dose, multiple doses or over an established period of time in an infusion. In example embodiments, these pharmaceutical compositions are administered as a single dose. In example embodiments, these pharmaceutical compositions are administered as a single subcutaneous injection.

For example, in some aspects, the present disclosure provides a method of treating Alzheimer's disease in a subject, comprising administering to the subject a pharmaceutical composition comprising from about 20 mg to about 200 mg of an anti-amyloid β antibody or an antigen-binding fragment thereof once about every 3-5 weeks.

In some aspects, the present disclosure provides a method of reducing amyloid plaque in a subject, comprising administering to the subject a pharmaceutical composition comprising from about 20 mg to about 200 mg of an anti-amyloid β antibody or an antigen-binding fragment thereof once about every 3-5 weeks.

In some aspects, the present disclosure provides a method of converting a subject from amyloid positive to amyloid negative, comprising administering to the subject a pharmaceutical composition comprising from about 20 mg to about 200 mg of an anti-amyloid β antibody or an antigen-binding fragment thereof once about every 3-5 weeks.

For example, in some aspects, the present disclosure provides a method of treating Alzheimer's disease in a subject, comprising administering to the subject a pharmaceutical composition comprising from about 20 mg to about 200 mg of an anti-amyloid β antibody or an antigen-binding fragment thereof once about every 3-5 weeks.

In some aspects, the present disclosure provides a method of reducing amyloid plaque in a subject, comprising administering to the subject a pharmaceutical composition comprising from about 20 mg to about 200 mg of an anti-amyloid β antibody or an antigen-binding fragment thereof once about every 3-5 weeks.

In some aspects, the present disclosure provides a method of converting a subject from amyloid positive to amyloid negative, comprising administering to the subject a pharmaceutical composition comprising from about 20 mg to about 200 mg of an anti-amyloid β antibody or an antigen-binding fragment thereof once about every 3-5 weeks.

In some embodiments, the method comprises administering to the subject a pharmaceutical composition comprising about 40 mg to about 50 mg of the anti-amyloid β antibody or an antigen-binding fragment thereof. In some embodiments, the method comprises administering to the subject a pharmaceutical composition comprising about 65 mg to about 75 mg of the anti-amyloid β antibody or an antigen-binding fragment thereof. In example embodiments, the method comprises administering to the subject a pharmaceutical composition comprising about 195 mg to about 205 mg of the anti-amyloid β antibody or an antigen-binding fragment thereof.

In example embodiments, the method comprises administering to the subject a pharmaceutical composition comprising about 45 mg of the anti-amyloid β antibody or an antigen-binding fragment thereof. In example embodiments, the method comprises administering to the subject a pharmaceutical composition comprising about 70 mg of the anti-amyloid β antibody or an antigen-binding fragment thereof. In example embodiments, the method comprises administering to the subject a pharmaceutical composition comprising about 200 mg of the anti-amyloid β antibody or an antigen-binding fragment thereof. In example embodiments, these pharmaceutical compositions are administered as a single subcutaneous injection once about every 3-5 weeks (e.g., once about every 4 weeks).

Several methods of preparing and administering anti-Aβ antibodies, or antigen-binding fragments, variants, or derivatives thereof to a subject in need thereof are known. The route of administration of an anti-Aβ antibody, or antigen-binding fragment, variant, or derivative thereof, can be, for example, peripheral, oral, central (e.g., intrathecal, intracranial), parenteral, by inhalation or topical.

As discussed herein, anti-Aβ antibodies, or antigen-binding fragments, variants, or derivatives thereof can be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an anti-Aβ antibody, or antigen-binding fragment, variant, or derivative thereof, shall be held to mean an amount sufficient to achieve effective binding to a target and to achieve a benefit, e.g., reduce brain amyloid plaques without affecting vascular amyloid, or minimizes the occurrence of microhemorrhage during chronic dosing of the anti-Aβ antibody or antigen-binding fragment thereof. In some embodiments, an anti-Aβ antibody or antigen-binding fragment, variant, or derivative thereof can cross the blood-brain barrier in an effective amount to reduce brain amyloid plaques.

The pharmaceutical compositions used in this disclosure comprise pharmaceutically acceptable carriers, including, e.g., ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

Prevention of the action of microorganisms can be achieved by various antibacterial and antifungal agents, for example, parabens, chlorobutanol, phenol, ascorbic acid, thimerosal and the like. In many cases, isotonic agents can be included, for example, sugars, polyalcohols or salts in the composition. Prolonged absorption of the injectable compositions can be brought about by including in the composition an agent which delays absorption, for example, aluminum monostearate and gelatin.

Parenteral formulations can be a single bolus dose, an infusion or a loading bolus dose followed with a maintenance dose. These compositions can be administered at specific fixed or variable intervals, e.g., once a day, or on an “as needed” basis.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like. Furthermore, the pharmaceutical composition of the disclosure can comprise further agents such as dopamine or psychopharmacologic drugs, depending on the intended use of the pharmaceutical composition.

The amount of an anti-Aβ antibody, or fragment, variant, or derivative thereof, to be combined with the carrier materials to produce a single dosage form will vary depending upon the host treated and the particular mode of administration. The composition can be administered as a single dose, multiple doses or over an established period of time in an infusion. Dosage regimens also can be adjusted to provide the optimum desired response (e.g., a therapeutic or prophylactic response).

The term “peripheral administration” as used herein includes, e.g., intravenous, intraarterial, intraperitoneal, intramuscular, subcutaneous, intranasal, intra-ocular/vitreal, rectal, or vaginal administration. While all these forms of administration are clearly contemplated as being within the scope of the disclosure, an example of a form for administration would be a solution for injection, in particular for subcutaneous, intravenous or intraarterial injection or drip. A suitable pharmaceutical composition for injection can comprise a buffer, a surfactant, optionally a stabilizer agent, etc. Preparations for peripheral administration include sterile aqueous or non-aqueous solutions, suspensions, and emulsions. Preservatives and other additives can also be present such as, for example, antimicrobials, antioxidants, chelating agents, and inert gases and the like.

Therapeutic compositions of the disclosure are typically substantially pure from undesired contaminants. This means that the agent is typically at least 50% w/w pure of interfering proteins and other contaminants arising from its production or purification but does not exclude the possibility that the agent is combined with an excess of pharmaceutical acceptable carrier(s) or other vehicle intended to facilitate its use. Sometimes monoclonal antibodies (or other therapeutic agents) are at least 60%, 70%, 80%, 90%, 95% or 99% w/w pure of interfering proteins and contaminants from production or purification.

### Pharmacokinetic Endpoints

The present disclosure provides dosing regimens for anti-amyloid β antibodies that are designed to achieve drug exposure profiles in subjects that are suitable for the clearance of amyloid plaque and/or treatment of neurodegenerative diseases (e.g., Alzheimer's disease). To this end, the present disclosure provides methods of administering anti-amyloid β antibodies to achieve particular pharmacokinetic endpoints in subjects, including, for example, values of the following parameters that are suitable for plaque clearance and/or treatment of the disease: average concentration over the dosing interval (Cₐᵥₑ), steady state concentration over the dosing interval (Cₛₛ), maximum concentration over the dosing interval (Cₘₐₓ), area under the concentration-time curve from time zero to infinity AUC0-0, and the area under the concentration-time curve for dosing interval (AUC₀₋ₜₐᵤ). In various embodiments, these pharmacokinetic endpoints can be assessed in a number of bodily fluids collected from the subject, including, for example, whole blood, blood serum, blood plasma, and/or CSF.

### Maximum Drug Concentration (Cₘₐₓ)

Thus, in another aspect, the present disclosure provides a method of treating Alzheimer's disease in a subject, comprising subcutaneously administering to the subject a dose of an anti-Aβ antibody sufficient to achieve a Cₘₐₓ value (steady state Cₘₐₓ value) of about 30 μg/mL to about 60 μg/mL. In another aspect, the present disclosure provides a method of reducing amyloid plaque in a subject, comprising subcutaneously administering to the subject a dose of an anti-Aβ antibody sufficient to achieve a Cₘₐₓ value of about 30 μg/mL to about 60 μg/mL. In another aspect, the present disclosure provides a method of converting a subject from amyloid positive to amyloid negative, comprising subcutaneously administering to the subject a dose of an anti-Aβ antibody sufficient to achieve a Cₘₐₓ value of about 30 μg/mL to about 60 μg/mL. In example embodiments, the anti-Aβ antibody comprises a heavy chain of SEQ ID NO: 101, with or without the C-terminal lysine, and a light chain of SEQ ID NO: 102.

In some embodiments, treating comprises achieving a Cₘₐₓ of anti-amyloid β the anti-amyloid β antibody or antigen binding fragment thereof in the subject of about 30 μg/mL to about 60 μg/mL (e.g., about 35 μg/mL to about 60 μg/mL, about 40 μg/mL to about 60 μg/mL, about 45 μg/mL to about 60 μg/mL, about 30 μg/mL to about 55 μg/mL, about 35 μg/mL to about 55 μg/mL, about 30 μg/mL to about 50 μg/mL, or about 35 μg/mL to about 50 μg/mL). In some embodiments, the Cₘₐₓ value is a steady state serum Cₘₐₓ value. In some embodiments, the Cₘₐₓ value is a steady state plasma Cₘₐₓ value.

### Average Drug Concentration (Cₐᵥₑ)

In another aspect, the present disclosure provides a method of treating Alzheimer's disease in a subject, comprising subcutaneously administering to the subject a dose of an anti-Aβ antibody sufficient to achieve a serum Cₐᵥₑ value of about 20 g/mL to about 40 μg/mL In another aspect, the present disclosure provides a method of reducing amyloid plaque in a subject, comprising subcutaneously administering to the subject a dose of an anti-Aβ antibody sufficient to achieve a serum Cₐᵥₑ value of about 20 μg/mL to about 40 μg/mL. In another aspect, the present disclosure provides a method of converting a subject from amyloid positive to amyloid negative, comprising subcutaneously administering to the subject a dose of an anti-Aβ antibody sufficient to achieve a serum Cₐᵥₑ value of about 20 μg/mL to about 40 μg/mL In example embodiments, the anti-Aβ antibody comprises a heavy chain of SEQ ID NO: 101, with or without the C-terminal lysine, and a light chain of SEQ ID NO: 102.

In some embodiments, treating comprises achieving a Cₘₐₓ of the anti-amyloid β antibody or antigen binding fragment thereof in the subject of about 20 μg/mL to about 40 μg/mL (e.g., about 23 μg/mL to about 40 μg/mL, about 25 μg/mL to about 40 μg/mL, about 28 μg/mL to about 40 μg/mL, about 30 μg/mL to about 40 μg/mL, about 35 μg/mL to about 40 μg/mL, about 20 μg/mL to about 38 μg/mL, about 23 μg/mL to about 38 μg/mL, about 25 μg/mL to about 38 μg/mL, about 28 μg/mL to about 38 μg/mL, about 20 μg/mL to about 35 μg/mL, about 20 μg/mL to about 30 μg/mL, or about 25 μg/mL to about 30 μg/mL). In some embodiments, the Cₐᵥₑ value is a steady state serum Cₐᵥₑ value. In some embodiments, the Cₐᵥₑ value is a steady state plasma Cₐᵥₑ value.

Area Under the Concentration-Time Curve for the Dosing Interval (AUC₀₋ₜₐᵤ)

In another aspect, the present disclosure provides a method of treating Alzheimer's disease in a subject, comprising subcutaneously administering to the subject a dose of an anti-Aβ antibody sufficient to achieve an area under the concentration-time curve for the dosing interval (AUC₀₋ₜₐᵤ) value of about 15,000 hr*ug/mL to about 30,000 hr*ug/mL. In another aspect, the present disclosure provides a method of reducing amyloid plaque in a subject, comprising subcutaneously administering to the subject a dose of an anti-Aβ antibody sufficient to achieve a AUC₀₋ₜₐᵤ value of about 15,000 hr*ug/mL to about 30,000 hr*ug/mL. In another aspect, the present disclosure provides a method of converting a subject from amyloid positive to amyloid negative, comprising subcutaneously administering to the subject a dose of an anti-Aβ antibody sufficient to achieve a AUC₀₋ₜₐᵤ value of about 15,000 hr*ug/mL to about 30,000 hr*ug/mL. In example embodiments, the anti-Aβ antibody comprising a heavy chain of SEQ ID NO: 101, with or without the C-terminal lysine, and a light chain of SEQ ID NO: 102.

In some embodiments, treating comprises achieving an AUC₀₋ₜₐᵤ value (steady state AUC₀₋ₜₐᵤ value) of the anti-amyloid β antibody or antigen binding fragment thereof in the subject is about 15,000 hr*ug/mL to about 30,000 hr*ug/mL (e.g., 16,000 hr*ug/mL to about 30,000 hr*ug/mL, 18,000 hr*ug/mL to about 30,000 hr*ug/mL, 20,000 hr*ug/mL to about 30,000 hr*ug/mL, 22,000 hr*ug/mL to about 30,000 hr*ug/mL, or 25,000 hr*ug/mL to about 30,000 hr*ug/mL). In some embodiments, treating comprises achieving an AUC₀₋ₜₐᵤ value (steady state AUC₀₋ₜₐᵤ value) of the anti-amyloid β antibody or antigen binding fragment thereof in the subject is about 15,000 hr*ug/mL to about 25,000 hr*ug/mL (e.g., 16,000 hr*ug/mL to about 25,000 hr*ug/mL, 18,000 hr*ug/mL to about 25,000 hr*ug/mL, 20,000 hr*ug/mL to about 25,000 hr*ug/mL, or 22,000 hr*ug/mL to about 25,000 hr*ug/mL). In some embodiments, the AUC₀₋ₜₐᵤ value is a steady state serum AUC₀₋ₜₐᵤ value. In some embodiments, the AUC₀₋ₜₐᵤ value is a steady state plasma AUC₀₋ₜₐᵤ value.

### Amyloid Plaque Clearance

The present disclosure further provides the use of anti-amyloid β antibodies to reduce amyloid plaque in a subject. Amyloid plaque reduction has been shown to correlate with slowing of cognitive decline during treatment with anti-amyloid β antibodies. See, e.g., M. Shi, et al., Impact of Anti-amyloid-β Monoclonal Antibodies on the Pathology and Clinical Profile of Alzheimer's Disease: A Focus on Aducanumab and Lecanemab. 14 FRONT. AGING NEUROSCI. 1 (2022); C. H. van Dyck, et al., Lecanemab in Early Alzheimer's Disease 388 N. ENGL. J. MED. 9 (2023). Indeed, the FDA granted accelerated approval to both aducanumab and lecanemab based on plaque reduction data from clinical trials.

Thus, in another aspect, the present disclosure provides a method of treating Alzheimer's disease in a subject having amyloid plaque, the method comprising:
(a) administering to the subject a composition comprising from about 20 mg to about 200 mg of an anti-amyloid β antibody once about every 4 weeks, the anti-amyloid β antibody comprising a heavy chain of SEQ ID NO: 101, with or without the C-terminal lysine, and a light chain of SEQ ID NO: 102; and
(b) reducing the amyloid plaque in the subject.

Detection of brain amyloid plaques is conducted by methods known to one of skill in the art. In some embodiments, the method further comprises assessing the amyloid by Positron Emission Tomography (PET) imaging. In some embodiments, the amyloid reduction is determined by PET. PET imaging agents are known to one of skill in the art and include 18F-florbetapir, florbetaben F18, and flutemetamol F18. In some embodiments, amyloid plaque, as measured by PET, is quantified by a composite standard uptake value ratio (SUVR). In some embodiments, amyloid plaque, as measured by PET, is calculated using the Centiloid scale. In some embodiments, change in amyloid plaque burden is measured by change in SUVR over time. In some embodiments, change in amyloid plaque burden is measured by change in Centiloid over time. See, Navitsky M, Joshi A D, Kennedy I, et al., Standardization of amyloid quantitation with florbetapir standardized uptake value ratios to the Centiloid scale, Alzheimers Dement 2018 14:1565-71 and Oshi A D, Pontecorvo M J, Lu M, et al., A Semiautomated Method for Quantification of F 18 Florbetapir PET Images, J Nucl Med. 2015; 56 (11): 1736-41.

Thus, in another aspect, the present disclosure provides a method of reducing amyloid plaque in a subject, the method comprising:
(a) administering to the subject a composition comprising from about 20 mg to about 200 mg (e.g., about 45 mg, about 70 mg, about 150 mg, or about 200 mg) of an anti-amyloid β antibody once about every 4 weeks, the anti-amyloid β antibody comprising a heavy chain of SEQ ID NO: 101, with or without the C-terminal lysine, and a light chain of SEQ ID NO: 102; and
(b) reducing the amyloid plaque in the subject, as determined by PET.

In another aspect, the present disclosure provides a method of treating Alzheimer's disease in a subject, comprising:
(a) observing a first amyloid plaque value obtained from a first PET scan of the subject;
(b) subcutaneously administering to the subject a composition comprising from about 20 mg to about 200 mg (e.g., about 45 mg, about 70 mg, about 150 mg, or about 200 mg) of an anti-amyloid β antibody once about every 3-5 weeks, the anti-amyloid β antibody comprising a heavy chain of SEQ ID NO: 101, with or without the C-terminal lysine, and a light chain of SEQ ID NO: 102; or
(c) observing a second amyloid plaque value obtained from a second PET scan of the subject; and
(d) comparing the first amyloid plaque value to the second amyloid plaque value, thereby observing a reduction in amyloid plaque in the subject.

In another aspect, the present disclosure provides a method of treating Alzheimer's disease in a subject having amyloid plaque, the method comprising:
(a) performing a first PET scan on the subject, thereby observing a first amyloid plaque value;
(b) administering to the subject a composition comprising from about 20 mg to about 200 mg (e.g., about 45 mg, about 70 mg, about 150 mg, or about 200 mg) of an anti-amyloid β antibody once about every 3-5 weeks; the anti-amyloid β antibody comprising a heavy chain of SEQ ID NO: 101, with or without the C-terminal lysine, and a light chain of SEQ ID NO: 102;
(c) performing a second PET scan on the subject, thereby observing a second amyloid plaque value; and
(d) comparing the first amyloid plaque value to the second amyloid plaque value, thereby observing a reduction in amyloid plaque in the subject.

In another aspect, the present disclosure provides a method of reducing amyloid plaque in a subject, comprising:
(a) observing a first amyloid plaque value obtained from a first PET scan of the subject;
(b) subcutaneously administering to the subject a composition comprising from about 20 mg to about 200 mg (e.g., about 45 mg, about 70 mg, about 150 mg, or about 200 mg) of an anti-amyloid β antibody once about every 3-5 weeks, the anti-amyloid β antibody comprising a heavy chain of SEQ ID NO: 101, with or without the C-terminal lysine, and a light chain of SEQ ID NO: 102; or
(c) observing a second amyloid plaque value obtained from a second PET scan of the subject; and
(d) comparing the first amyloid plaque value to the second amyloid plaque value, thereby observing a reduction in amyloid plaque in the subject.

In another aspect, the present disclosure provides a method of reducing amyloid plaque in a subject having amyloid plaque, the method comprising:
(a) performing a first PET scan on the subject, thereby observing a first amyloid plaque value;
(b) administering to the subject a composition comprising from about 20 mg to about 200 mg (e.g., about 45 mg, about 70 mg, about 150 mg, or about 200 mg) of an anti-amyloid β antibody once about every 3-5 weeks; the anti-amyloid β antibody comprising a heavy chain of SEQ ID NO: 101, with or without the C-terminal lysine, and a light chain of SEQ ID NO: 102;
(c) performing a second PET scan on the subject, thereby observing a second amyloid plaque value; and
(d) comparing the first amyloid plaque value to the second amyloid plaque value, thereby observing a reduction in amyloid plaque in the subject.

In some embodiments, treating comprises a reduction in amyloid beta plaque (i.e., brain amyloid beta plaque) in the subject. In some embodiments, the treatment results in the subject achieving a reduction of brain amyloid beta plaque. In some embodiments, the subject achieves a reduction of brain amyloid beta plaque as assessed by PET.

In some embodiments, the treatment results in the patient achieving a reduction of brain amyloid beta plaque. In some embodiments, the patient achieves a reduction of brain amyloid beta plaque as assessed by Positron Emission Tomography (PET). In some embodiments, the reduction of brain amyloid beta plaque comprises a reduction by at least about 10 centiloids (e.g, at least about 15 centiloids, at least about 20 centiloids, at least about 25 centiloids, or at least about 30 centiloids) compared to baseline. In some embodiments, the reduction of brain amyloid beta plaque comprises a reduction by at least about 30 centiloids (e.g, at least about 35 centiloids, at least about 40 centiloids, at least about 45 centiloids, at least about 50 centiloids) compared to baseline. In some embodiments, the reduction of brain amyloid beta plaque occurs after about 6 months (e.g., after about 24 weeks) of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction of brain amyloid beta plaque comprises a reduction by at least about 10 centiloids (e.g, at least about 15 centiloids, at least about 20 centiloids, at least about 25 centiloids, or at least about 30 centiloids). In some embodiments, the reduction of brain amyloid beta plaque comprises a reduction by at least about 30 centiloids after 6 months of treatment (e.g, at least about 35 centiloids, at least about 40 centiloids, at least about 45 centiloids, at least about 50 centiloids, at least about 55 centiloids, or at least about 60 centiloids). In some embodiments, the reduction of brain amyloid beta plaque is achieved after about 6 months of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction of brain amyloid beta plaque comprises a reduction by about 10 centiloids to about 90 centiloids (e.g, about 20 centiloids to about 90 centiloids, about 30 centiloids to about 90 centiloids, about 40 centiloids to about 90 centiloids, or about 50 centiloids to about 90 centiloids). In some embodiments, the reduction of brain amyloid beta plaque comprises a reduction by about 10 centiloids to about 80 centiloids (e.g., about 20 centiloids to about 80 centiloids, about 30 centiloids to about 80 centiloids, about 40 centiloids to about 80 centiloids, or about 50 centiloids to about 80 centiloids). In some embodiments, the reduction of brain amyloid beta plaque comprises a reduction by about 10 centiloids to about 70 centiloids (e.g., about 20 centiloids to about 70 centiloids, about 30 centiloids to about 70 centiloids, about 40 centiloids to about 70 centiloids, or about 50 centiloids to about 70 centiloids). In some embodiments, the reduction of brain amyloid beta plaque comprises a reduction by about 10 centiloids to about 60 centiloids (e.g., about 20 centiloids to about 60 centiloids, about 30 centiloids to about 60 centiloids, about 40 centiloids to about 60 centiloids, or about 50 centiloids to about 60 centiloids). In some embodiments, the reduction of brain amyloid beta plaque is achieved after about 6 months of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction of brain amyloid beta plaque comprises a reduction by at least about 10 centiloids after about 6 months of treatment (e.g, at least about 15 centiloids, at least about 20 centiloids, at least about 25 centiloids, or at least about 30 centiloids). In some embodiments, the reduction of brain amyloid beta plaque comprises a reduction by at least about 30 centiloids after about 6 months of treatment (e.g, at least about 35 centiloids, at least about 40 centiloids, at least about 45 centiloids, or at least about 50 centiloids). In some embodiments, the reduction of brain amyloid beta plaque comprises a reduction by about 10 centiloids, about 15 centiloids, about 20 centiloids, about 25 centiloids, about 30 centiloids, about 35 centiloids, about 40 centiloids, about 45 centiloids, about 50 centiloids, about 55 centiloids, about 60 centiloids, about 65 centiloids, about 70 centiloids, about 75 centiloids, about 80 centiloids, about 85 centiloids, about 90 centiloids or about 95 centiloids. In some embodiments, the reduction of brain amyloid beta plaque is achieved after about 6 months of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction of brain amyloid beta plaque comprises a reduction by about 10 centiloids to about 80 centiloids after about 6 months of treatment (e.g, about 20 centiloids to about 80 centiloids, about 30 centiloids to about 80 centiloids, about 10 centiloids to about 60 centiloids, or about 10 centiloids to about 50 centiloids). In some embodiments, the reduction of brain amyloid beta plaque comprises a reduction by about 30 centiloids to about 70 centiloids after about 6 months of treatment (e.g., about 40 centiloids to about 70 centiloids, about 50 centiloids to about 70 centiloids, about 30 centiloids to about 60 centiloids, or about 30 centiloids to about 50 centiloids).

In some embodiments, the reduction of brain amyloid beta plaque comprises a reduction by at least about 25 centiloids after about 12 months of treatment (e.g, at least about 35 centiloids, at least about 40 centiloids, at least about 45 centiloids, or at least about 50 centiloids). In some embodiments, the reduction of brain amyloid beta plaque comprises a reduction by at least about 40 centiloids after about 12 months of treatment (e.g, at least about 45 centiloids, at least about 50 centiloids, at least about 55 centiloids, or at least about 60 centiloids).

In some embodiments, the reduction of brain amyloid beta plaque comprises a reduction by about 20 centiloids to about 90 centiloids after about 12 months of treatment (e.g, about 30 centiloids to about 90 centiloids, about 40 centiloids to about 90 centiloids, about 20 centiloids to about 80 centiloids, or about 20 centiloids to about 70 centiloids). In some embodiments, the reduction of brain amyloid beta plaque comprises a reduction by about 45 centiloids to about 80 centiloids after about 12 months of treatment (e.g., about 50 centiloids to about 80 centiloids, about 55 centiloids to about 80 centiloids, about 45 centiloids to about 75 centiloids, or about 45 centiloids to about 70 centiloids).

In some embodiments, the reduction of brain amyloid beta plaque comprises a reduction by at least about 35 centiloids after about 18 months of treatment (e.g, at least about 40 centiloids, at least about 45 centiloids, at least about 50 centiloids, or at least about 55 centiloids). In some embodiments, the reduction of brain amyloid beta plaque comprises a reduction by at least about 50 centiloids after about 18 months of treatment (e.g, at least about 55 centiloids, at least about 60 centiloids, at least about 65 centiloids, or at least about 70 centiloids).

In some embodiments, the reduction of brain amyloid beta plaque comprises a reduction by about 30 centiloids to about 100 centiloids after about 18 months of treatment (e.g, about 40 centiloids to about 100 centiloids, about 50 centiloids to about 100 centiloids, about 30 centiloids to about 95 centiloids, or about 30 centiloids to about 90 centiloids). In some embodiments, the reduction of brain amyloid beta plaque comprises a reduction by about 50 centiloids to about 85 centiloids after about 18 months of treatment (e.g., about 65 centiloids to about 85 centiloids, about 70 centiloids to about 85 centiloids, about 50 centiloids to about 80 centiloids, or about 50 centiloids to about 75 centiloids).

In some embodiments, the treatment results in the reduction of brain amyloid beta plaque in the patient comprises a reduction by at least 20% (e.g. at least 22%, at least 25%, at least 27%, at least 30%, at least 32%, at least 35%, or at least 37%) compared to baseline. at least 40% (e.g. at least 41%, at least 42%, at least 43%, at least 44%, at least 45%, at least 46%, at least 47%, at least 48%, at least 49%, or at least 50%) compared to baseline.

In some embodiments, treating comprises a reduction in brain amyloid beta plaque. In some embodiments, the reduction in brain amyloid beta plaque comprises a reduction of at least about 30% (e.g., at least about 40%, at least about 45%, at least about 50%, at least about 55%, at least about 60%, at least about 65%, or at least about 70%). In some embodiments, the reduction of brain amyloid beta plaque comprises a reduction of about 30% to about 100% (e.g., about 40% to about 100%, about 50% to about 100%, about 60% to about 100%, or about 70% to about 100%). In some embodiments, reduction of brain amyloid beta plaque in the subject comprises a reduction of about 30% to about 90% (e.g., about 40% to about 90%, about 50% to about 90%, about 60% to about 90%, or about 70% to about 90%,). In some embodiments, the reduction of brain amyloid beta plaque comprises a reduction of about 30% to about 80% (e.g., about 40% to about 80%, about 50% to about 80%, or about 60% to about 80%). In some embodiments, the reduction of brain amyloid beta plaque comprises a reduction of about 30% to about 70% (e.g., about 40% to about 70%, or about 50% to about 70%). In some embodiments, the reduction of brain amyloid beta plaque comprises a reduction of about 25%, about 30%, about 35%, about 40%, about 45%, about 50%, about 55%, about 60%, about 65%, about 70%, about 75%, about 80%, about 85%, about 90%, about 95%, or about 100%. In some embodiments, the reduction of brain amyloid beta plaque is achieved after about 6 months of treatment. In some embodiments, the reduction of brain amyloid beta plaque is achieved after about 12 months of treatment. In some embodiments, the reduction of brain amyloid beta plaque is achieved after about 18 months of treatment.

In some embodiments, the reduction in brain amyloid beta plaque comprises a reduction of at least about 20% after about 6 months of treatment (e.g., at least 25%, at least about 30%, at least about 35%, at least about 40%, at least about 45%, or at least about 50%). In some embodiments, the reduction of brain amyloid beta plaque comprises a reduction of about 20% to about 90% after about 6 months of treatment (e.g., about 30% to about 90%, about 40% to about 90%, or about 50% to about 90%). In some embodiments, reduction of brain amyloid beta plaque in the subject comprises a reduction of about 30% to about 70% after about 6 months of treatment (e.g., about 35% to about 70%, about 40% to about 70%, about 30% to about 65%, or about 30% to about 60%).

In some embodiments, the reduction in brain amyloid beta plaque comprises a reduction of at least about 30% after about 12 months of treatment (e.g., at least 35%, at least about 40%, at least about 45%, at least about 50%, at least about 55%, or at least about 60%). In some embodiments, the reduction of brain amyloid beta plaque comprises a reduction of about 30% to about 100% after about 12 months of treatment (e.g., about 40% to about 100%, about 50% to about 100%, or about 50% to about 90%). In some embodiments, reduction of brain amyloid beta plaque in the subject comprises a reduction of about 60% to about 100% after about 12 months of treatment (e.g., about 65% to about 100%, about 70% to about 100%, about 65% to about 95%, or about 65% to about 90%).

In some embodiments, the reduction in brain amyloid beta plaque comprises a reduction of at least about 40% after about 18 months of treatment (e.g., at least 45%, at least about 50%, at least about 55%, at least about 60%, at least about 65%, or at least about 70%). In some embodiments, the reduction of brain amyloid beta plaque comprises a reduction of about 40% to about 100% after about 18 months of treatment (e.g., about 50% to about 100%, about 60% to about 100%, or about 50% to about 90%). In some embodiments, reduction of brain amyloid beta plaque in the subject comprises a reduction of about 65% to about 100% after about 18 months of treatment (e.g., about 70% to about 100%, about 75% to about 100%, about 65% to about 95%, or about 65% to about 90%).

In some embodiments, the treatment results in the reduction of brain amyloid beta plaque in the patient comprises a reduction by at least 0.05 PET Standard Update Value Ratio (“SUVr”) units (e.g., at least 0.10 PET SUVr units, at least 0.15 PET SUVr units, at least 0.20 PET SUVr units, or at least 0.25 PET SUVr unit) compared to baseline. In some embodiments, the reduction of brain amyloid beta plaque comprises a reduction by at least 0.25 PET SUVr units (e.g., at least 0.30 PET SUVr units, at least 0.35 PET SUVr units, or at least 0.40 PET SUVr units, or at least 0.45 PET SUVr) compared to baseline. In some embodiments, the reduction of brain amyloid beta plaque comprises a reduction by at least 0.50 PET SUVr units (e.g., at least 0.55 PET SUVr units, at least 0.60 PET SUVr units, or at least 0.65 PET SUVr units, or at least 0.70 PET SUVr) compared to baseline.

In some embodiments, the reduction of brain amyloid beta plaque occurs after about 6 months (e.g., after about 24 weeks) of treatment. In some embodiments, the reduction of brain amyloid beta plaque occurs after about 12 months (e.g., after about 48 weeks) of treatment. In some embodiments, the reduction of brain amyloid beta plaque occurs after about 18 months (e.g., after about 72 weeks) of treatment.

In some embodiments, the treatment results in the patient achieving amyloid negative status. In some embodiments, the patient is converted from amyloid positive status to amyloid negative status.

In another aspect, the present disclosure provides a method of converting a subject from amyloid positive to amyloid negative, the method comprising:
(a) administering to the subject a composition comprising from about 20 mg to about 200 mg of an anti-amyloid β antibody once about every 4 weeks, the anti-amyloid β antibody comprising a heavy chain of SEQ ID NO: 101, with or without the C-terminal lysine, and a light chain of SEQ ID NO: 102; and
(b) reducing the amyloid plaque in the subject, such that the subject is amyloid negative, as determined by PET.

In some embodiments, treating comprises at least about 10% (e.g., at least about 15%, at least about 20%, at least about 25%, at least about 30%, at least about 45%, or at least about 50%) of subjects being converted from amyloid positive status to amyloid negative status. In some embodiments, treating comprises about 10% to about 90% (e.g., about 20% to about 90%, about 30% to about 90%, about 40% to about 90%, about 10% to about 80%, about 10% to about 70%, or about 10% to about 60%) of subjects being converted from amyloid positive status to amyloid negative status. In some embodiments, treating comprises about 30% to about 80% (e.g., about 40% to about 80%, about 50% to about 80%, about 30% to about 70%, or about 30% to about 60%) of subjects being converted from amyloid positive status to amyloid negative status. In some embodiments, amyloid positive and/or amyloid negative status is assessed by PET. In some embodiments, the achievement of amyloid negative status occurs after about 6 months (e.g., about 24 weeks) of treatment. In some embodiments, the achievement of amyloid negative status occurs after about 12 months (e.g., about 48 weeks) of treatment. In some embodiments, the achievement of amyloid negative status occurs after about 18 months (e.g., about 72 weeks) of treatment.

In some embodiments, treating comprises at least about 5% (e.g., at least about 10%, at least about 15%, at least about 20%, or at least about 25%) of subjects being converted from amyloid positive status to amyloid negative status after about 6 months. In some embodiments, treating comprises about 5% to about 50% (e.g., about 10% to about 50%, about 20% to about 50%, about 10% to about 45%, or about 10% to about 40%) of subjects being converted from amyloid positive status to amyloid negative status after about 6 months. In some embodiments, treating comprises about 10% to about 40% (e.g., about 15% to about 40%, about 20% to about 40%, about 10% to about 35%, or about 10% to about 30%) of subjects being converted from amyloid positive status to amyloid negative status after about 6 months.

In some embodiments, treating comprises at least about 15% (e.g., at least about 20%, at least about 25%, at least about 30%, or at least about 35%) of subjects being converted from amyloid positive status to amyloid negative status after about 12 months. In some embodiments, treating comprises about 15% to about 80% (e.g., about 20% to about 80%, about 30% to about 80%, about 15% to about 75%, or about 15% to about 70%) of subjects being converted from amyloid positive status to amyloid negative status after about 12 months. In some embodiments, treating comprises about 30% to about 60% (e.g., about 35% to about 60%, about 40% to about 60%, about 30% to about 55%, or about 30% to about 50%) of subjects being converted from amyloid positive status to amyloid negative status after about 12 months.

In some embodiments, treating comprises at least about 25% (e.g., at least about 30%, at least about 35%, at least about 40%, or at least about 45%) of subjects being converted from amyloid positive status to amyloid negative status after about 18 months. In some embodiments, treating comprises about 25% to about 100% (e.g., about 30% to about 100%, about 40% to about 100%, about 25% to about 95%, or about 25% to about 90%) of subjects being converted from amyloid positive status to amyloid negative status after about 18 months. In some embodiments, treating comprises about 40% to about 80% (e.g., about 45% to about 80%, about 50% to about 80%, about 40% to about 75%, or about 40% to about 70%) of subjects being converted from amyloid positive status to amyloid negative status after about 18 months.

In some embodiments, brain amyloid beta plaque in a subject is measured about 3 months, 6 months, 12 months, and/or 18 months after treatment is initiated. In some embodiments, brain amyloid beta plaque in a subject is measured, about once a month, about once every 3 months, about once every 6 months, or about once every year after treatment is initiated.

### Cognitive Decline

Cognitive decline is a feature of neurodegenerative diseases, including Alzheimer's disease. Initial stages of cognitive decline in Alzheimer's disease may manifest as forgetfulness. However, as the disease progresses, the effects of cognitive decline more broadly affect the patient's life and behavior, affecting executive function, language skills, and visuospatial processing. This, in turn, may lead to issues with decision-making, problem-solving, and independent living. Finally, Alzheimer's disease culminates in significant cognitive decline and dementia, leading to impairments in basic memory retention affected and simple daily activities.

Recent evidence has demonstrated a link between reduction in brain amyloid load and slowing of cognitive decline in Alzheimer's patients. See, e.g., Y. Zhang, et al., Amyloid β-based therapy for Alzheimer's disease: challenges, successes and future, 8 SIGNAL TRANSDUCTION AND TARGETED THERAPY 248 (2023). For example, a phase 2 clinical trial of donanemab showed both a reduction in brain amyloid load and a slowing of cognitive decline. More recently, a phase 3 clinical trial of lecanemab also reported both a reduction in brain amyloid plaque and a slowing of cognitive decline. In light of this recent clinical evidence, a causal link is emerging between clearing amyloid plaque and slowing cognitive decline in patients.

In some embodiments, treating Alzheimer's disease may include the slowing, halting, and/or reversing of cognitive decline in subjects. In some embodiments, treating comprises slowing, halting, and/or reversing a decline in cognitive function. In another embodiment, treating comprises a reduction (e.g., slowing or halting) in a decline in cognitive function. In another embodiment, treating comprises slowing a decline in cognitive function. In another embodiment, treating comprises halting a decline in cognitive function. In another embodiment, treating comprises reversing a decline in cognitive function.

Various cognitive assessment tools are available and can be used in conjunction with methods of the present disclosure, including, for example, Mini-Mental State Exam (MMSE), Alzheimer's Disease Composite Score (ADCOMS), Alzheimer's Disease Assessment Scale-Cognitive (ADAS-COG) (including, for example a 14-item Alzheimer's Disease Assessment Scale-Cognitive (ADAS-Cog14)), Activities of Daily Living for Mild Cognitive Impairment (ADCS-ADL-MCI), Clinician Interview-Based Impression (CIBI), Neurological Test Battery (NTB), Disability Assessment for Dementia (DAD), Clinical Dementia Rating-sum of boxes (CDR-SB), Neuropsychiatric Inventory (NPI). In some embodiments, treating comprises slowing, halting and/or reversing of cognitive decline as assessed using one of these cognitive assessment tools. In another embodiment, methods of the present disclose further comprise monitoring the subject by at least one of these cognitive assessment tools.

In some embodiments, cognitive function is measured by at least one of the following CRD-SB, ADAS-Cog14, ADCOMS, and ADCS MCI-ADL. In some embodiments, cognitive function is measured using CRD-SB. In some embodiments, cognitive function in measured using ADAS-Cog14. In some embodiments, cognitive function is measured using ADCOMS. In some embodiments, cognitive function is measured using ADCS MCI-ADL.

In some embodiments, cognitive function is measured on multiple occasions, such as before administering the dosage and at week 4, week 16, 6 months, and/or 1 year after administering the dosage. In some embodiments, cognitive function is measured about 3 months, 6 months, 12 months, and/or 18 months after treatment is initiated. In some embodiments, cognitive function is measured, about once a month, about once every 3 months, about once every 6 months, or about once every year after treatment is initiated.

### Biomarker Modulation

In another aspect, the present disclosure provides a method of modulating a biomarker in a subject, comprising administering to the subject a composition comprising from about 20 mg to about 200 mg of an anti-amyloid β antibody once about every 3-5 weeks, the anti-amyloid β antibody comprising a heavy chain of SEQ ID NO: 101, with or without the C-terminal lysine, and a light chain of SEQ ID NO: 102. In another embodiment, the present disclosure provides a method of modulating a biomarker in a subject, comprising administering to the subject a composition comprising from about 100 mg to about 200 mg of an anti-amyloid β antibody twice about every 3-5 weeks (e.g., once about every 2 weeks), the anti-amyloid β antibody comprising a heavy chain of SEQ ID NO: 101, with or without the C-terminal lysine, and a light chain of SEQ ID NO: 102. In another embodiment, the present disclosure provides a method of modulating a biomarker in a subject, comprising administering to the subject a composition comprising from about 100 mg to about 200 mg of an anti-amyloid β antibody twice about every 4 weeks (e.g., once about every 2 weeks), the anti-amyloid β antibody comprising a heavy chain of SEQ ID NO: 101, with or without the C-terminal lysine, and a light chain of SEQ ID NO: 102.

### Amyloid Beta Ratio

In some embodiments, the biomarker comprises the ratio of Aβ 42/40 in the subject. The Aβ₄₂/Aβ₄₀ ratio (e.g., the ratio of amyloid β₄₂ to amyloid β₄₀ in CSF and/or blood) has been demonstrated to be associated with well-established indicators of AD, including amyloid PET and CSF biomarkers, with lower Aβ₄₂/Aβ₄₀ ratios (e.g., an Aβ42/40 ratio <0.150) corresponding to higher amyloid plaque burden. See, e.g., C. Delaby, et al., The Aβ₁₋₄₂/Aβ1-40 ratio in CSF is more strongly associated to tau markers and clinical progression than Aß1-42 alone, 14 ALZHEIMER'S RESEARCH & THERAPY 20 (2022); X. Chang, et al., A Review of Application of Aβ42/40 Ratio in Diagnosis and Prognosis of Alzheimer's Disease. 90 J. ALZHEIMER'S DISEASE 495 (2002). For example, total plasma Aβ₄₂/Aβ₄₀ ratio has demonstrated value in the identification of individuals suffering from mild cognitive impairment (MCI), in the prediction of progression to dementia, and in the detection of underlying AD pathology revealed by FDG-PET, Amyloid-PET and CSF biomarkers. See, e.g., V. Perez-Grijalba, et al., Plasma Aβ42/40 Ratio Detects Early Stages of Alzheimer's Disease and Correlates with CSF and Neuroimaging Biomarkers in the Aβ255 Study. 6 J. PREVENTION OF ALZHEIMER'S DISEASE 34, (2019). Thus, changes in Aβ₄₂/Aβ₄₀ ratio may be useful in following individuals throughout the course of treatment, e.g., with an increase in the Aβ₄₂/Aβ₄₀ ratio signaling a reduction in amyloid plaque burden during treatment.

Further, an emerging trend in the treatment of Alzheimer disease (AD) is the shift of the therapeutic target population from people with dementia or MCI to cognitively healthy people at risk of AD. This population of at-risk people is difficult to identify if β-amyloid (Aβ) positivity is the primary criterion for eligibility in clinical trials. Using this metric, the screening rate of failure (SRF) rises to >70% in this population. See, e.g., J. D. Doecke, et al., Total Aβ₄₂/Aβ₄₀ ratio in plasma predicts amyloid-PET status, independent of clinical AD diagnosis. 94 NEUROLOGY 1580 (2020). Aβ₄₂/Aβ₄₀ ratio may be useful in identifying this population and following them throughout treatment (e.g., prophylactic treatment).

Thus, the present disclosure provides method of modulating a ratio of Aβ 42/40 in a subject, comprising administering to the subject a composition comprising from about 20 mg to about 200 mg of an anti-amyloid β antibody once every 3-5 weeks, the anti-amyloid β antibody comprising a heavy chain of SEQ ID NO: 101, with or without the C-terminal lysine, and a light chain of SEQ ID NO: 102. In some embodiments, the modulating comprises increasing the ratio of Aβ 42/40 in the subject.

In another aspect, the present disclosure provides a method of increasing a ratio of Aβ 42/40 in a subject, comprising administering to the subject a composition comprising from about 20 mg to about 200 mg of an anti-amyloid β antibody once about every 3-5 weeks, the anti-amyloid β antibody comprising a heavy chain of SEQ ID NO: 101, with or without the C-terminal lysine, and a light chain of SEQ ID NO: 102.

In another aspect, the present disclosure provides a method of increasing a ratio of Aβ 42/40 in a subject, comprising administering to the subject a composition comprising from about 100 mg to about 200 mg of an anti-amyloid β antibody twice about every 3-5 weeks (e.g., once about every 2 weeks), the anti-amyloid β antibody comprising a heavy chain of SEQ ID NO: 101, with or without the C-terminal lysine, and a light chain of SEQ ID NO: 102.

A method of increasing a ratio of Aβ 42/40 in a subject, the method comprising:
(a) administering to the subject a composition comprising from about 20 mg to about 200 mg of an anti-amyloid β antibody once about every 3-5 weeks, the anti-amyloid β antibody comprising a heavy chain of SEQ ID NO: 101, with or without the C-terminal lysine, and a light chain of SEQ ID NO: 102; and
(b) determining an Aβ 42/40 ratio value derived from a sample collected from the subject, wherein the Aβ 42/40 ratio value demonstrates an increase in the ratio of Aβ 42/40 in the subject.

A method of increasing a ratio of Aβ 42/40 in a subject, the method comprising:
(a) administering to the subject a composition comprising from about 100 mg to about 200 mg of an anti-amyloid β antibody twice about every 3-5 weeks, the anti-amyloid β antibody comprising a heavy chain of SEQ ID NO: 101, with or without the C-terminal lysine, and a light chain of SEQ ID NO: 102; and
(b) determining an Aβ 42/40 ratio value derived from a sample collected from the subject, wherein the Aβ 42/40 ratio value demonstrates an increase in the ratio of Aβ 42/40 in the subject.

In some embodiments, the Aβ 42/40 ratio value increases at least about 10% (e.g., about 15%, about 20%, about 25%, about 30%, about 35%, or about 40%). In some embodiments, the Aβ 42/40 ratio value increases about 10% to about 150% (e.g., about 20% to about 150%, about 30% to about 150%, about 10% to about 120%, about 20% to about 120%, about 30% to about 120%, about 10% to about 100%, or about 20% to about 100%). In some embodiments, the Aβ 42/40 ratio value increases about 25% to about 100% (e.g., about 30% to about 100%, about 35% to about 100%, about 40% to about 100%, about 25% to about 90%, about 25% to about 80%, about 35% to about 90%, or about 35% to about 80%). In some embodiments, the Aβ 42/40 ratio value increases compared to baseline.

### Phospho-Tau

Phospho-tau (p-tau) species have emerged as the most promising biomarkers of Alzheimer's disease. See, e.g., S. Janelidze, et al., Head-to-head comparison of 10 plasma phospho-tau assays in prodromal Alzheimer's disease. 19 BRAIN 1591-1601 (2023). Hyperphosphorylation of tau is a hallmark of Alzheimer's disease pathology, leading to self-aggregation of p-tau bundles in diseased subjects. See, e.g., C.-X. Gong, K. Iqbal, Hyperphosphorylation of Microtubule-Associated Protein Tau: A Promising Therapeutic Target for Alzheimer Disease, 15 CURRENT MED. CHEM. 2331 (2009). Phospho-tau levels (e.g., in blood) correlate with amyloid beta (Aβ) pathology and disease severity, as well as with established cerebrospinal fluid (CSF) and neuroimaging biomarkers. See, e.g., Kac, P. R., et al. Diagnostic value of serum versus plasma phospho-tau for Alzheimer's disease. 14 ALZ RES THERAPY 65 (2022). Phospho-tau further differentiates biomarker-positive AD dementia from other dementias as well as Aβ-negative controls, thereby demonstrating specificity to AD versus non-AD neurodegenerative diseases. Thus, p-tau levels can inform clinical diagnosis and eligibility for therapies, including treatment with anti-amyloid β antibodies. Further, p-tau species have the capacity to help expand access to AD diagnostics worldwide, as p-tau species can be measured in blood samples, which, in contrast to cerebrospinal fluid, do not require lumbar puncture to acquire. See, e.g., F. Gonzalez-Ortiz, et al., Plasma phospho-tau in Alzheimer's disease: towards diagnostic and therapeutic trial applications. 18 MOL. NEURODEGENERATION 18 (2023).

Several phospho-tau species have been identified as biomarkers for Alzheimer's disease, including p181-tau, p212-tau, p217-tau, p231-tau, and p235-tau. For example, plasma values of p-tau181, p-tau217 and p-tau231 have demonstrated associations with in vivo pathological hallmarks and autopsy-verified diagnosis. Several of these p-tau species are highly accurate at detecting brain amyloidosis and predicting whether patients will progress to cognitive impairment and neurodegeneration. Further, p-tau levels have been shown to change in subjects undergoing anti-amyloid β therapy, correlating with amyloid clearance. See, e.g., F. Gonzalez-Ortiz (2023).

In some aspects, the present disclosure provides a method of modulating an amount of phospho-tau in a subject, comprising administering to the subject a composition comprising from about 20 mg to about 200 mg of an anti-amyloid β antibody thereof once about every 3-5 weeks, the anti-amyloid β antibody comprising a heavy chain of SEQ ID NO: 101, with or without the C-terminal lysine, and a light chain of SEQ ID NO: 102. In some aspects, the present disclosure provides a method of modulating an amount of phospho-tau in a subject, comprising administering to the subject a composition comprising from about 100 mg to about 200 mg of an anti-amyloid β antibody thereof twice about every 3-5 weeks (e.g., once about every 2 weeks), the anti-amyloid β antibody comprising a heavy chain of SEQ ID NO: 101, with or without the C-terminal lysine, and a light chain of SEQ ID NO: 102. In some embodiments, the modulating comprises increasing the amount of phospho-tau in the subject.

Thus, in some embodiments, the biomarker comprises a phospho-tau value. In some embodiments, the phospho-tau value comprises at least one of the following: a p181-tau value, a p212-tau value, p217-tau value, a p231-tau value, and a p235-tau value. In some embodiments, the phospho-tau value comprises a p181-tau value. In some embodiments, the phospho-tau value comprises a p212-tau value. In some embodiments, the phospho-tau value comprises a p217-tau value. In some embodiments, the phospho-tau value comprises a p231-tau value. In some embodiments, the phospho-tau value comprises a p235-tau value.

In some embodiments, the phospho-tau value decreases about 5% to about 50% (e.g., about 10% to about 50%, about 15% to about 50%, about 20% to about 50%, about 10% to about 45%, about 10% to about 40%, or about 10% to about 35%). In some embodiments, the phospho-tau value decreases about 10% to about 30% (e.g., about 15% to about 30%, about 20% to about 30%, about 10% to about 25%, about 15% to about 25%, or about 20% to about 30%). In some embodiments, the phospho-tau value decreases compared to baseline.

In some embodiments, the p181-tau value decreases about 5% to about 50% (e.g., about 10% to about 50%, about 15% to about 50%, about 20% to about 50%, about 10% to about 45%, about 10% to about 40%, or about 10% to about 35%). In some embodiments, the p181-tau value decreases about 10% to about 30% (e.g., about 15% to about 30%, about 20% to about 30%, about 10% to about 25%, about 15% to about 25%, or about 20% to about 30%). In some embodiments, the p181-tau value decreases compared to baseline.

In some embodiments, the p217-tau value decreases about 5% to about 50% (e.g., about 10% to about 50%, about 15% to about 50%, about 20% to about 50%, about 10% to about 45%, about 10% to about 40%, or about 10% to about 35%). In some embodiments, the p217-tau value decreases about 10% to about 30% (e.g., about 15% to about 30%, about 20% to about 30%, about 10% to about 25%, about 15% to about 25%, or about 20% to about 30%). In some embodiments, the p217-tau value decreases compared to baseline.

### Amyloid Related Imaging Abnormalities (ARIA)

Treatment with amyloid β-targeted passive immunotherapy with (including, for example, aducanumab (Aduhelm), lecanemab, donanemab, and gantenerumab) comes with a significant risk of amyloid-related imaging abnormality (ARIA). See, e.g., M. Filippi, et al., Amyloid-Related Imaging Abnormalities and β-Amyloid-Targeting Antibodies A Systematic Review. 79 JAMA NEUROL. 291 (2022). AIRA is the most common side effect of anti-amyloid β antibodies, and can be classified as ARIA-E (cerebral edema, involving the breakdown of the tight endothelial junctions of the blood-brain barrier and subsequent accumulation of fluid) and ARIA-H (cerebral microhemorrhages (mH), small haemorrhages on the brain that are often accompanied by hemosiderosis). ARIA-E can be associated with acute neuroinflammation and overwhelming of the perivascular clearance systems, and ARIA-H may be related to vascular amyloid clearance and subsequence weakening and/or rupture of small blood vessels. See, e.g., H. Hampel, et al., Amyloid-related imaging abnormalities (ARIA): radiological, biological and clinical characteristics, 146 BRAIN 4414 (2023).

While often asymptomatic and detected only via MRI, in some instances ARIA may be symptomatic. For example, ARIA has been shown to include a number of side effects, such as headache, worsening confusion, dizziness, visual disturbances, nausea, and seizures. Further, at least one fatality related to ARIA-E during aducanumab treatment and at least one fatality due to ARIA-H during donanemab treatment has been reported to date. See, e.g., C. G. Withington & R. S. Turner, Amyloid-Related Imaging Abnormalities With Anti-amyloid Antibodies for the Treatment of Dementia Due to Alzheimer's Disease. 13 FRONTIERS IN NEUROLOGY 1 (2022). The risk of ARIA-H increases with age and cerebrovascular disease, and ARIA rates are generally higher in ApoE4 homozygous patients (ApoE4 carriers) compared to either ApoE4 non-carriers or ApoE4 heterozygous patients Furthermore, increased risk of ARIA-E has been observed at treatment initiation, and corresponds with higher dosage and with >4 of microhemorrhages on a baseline MRI.

In some embodiments, the treatment comprises a risk of ARIA-E that is less than about 70% (e.g., less than about 65%, less than about 60%, less than about 55%, or less than about 50%). In some embodiments, the treatment comprises a risk of ARIA-E that is less than about 45% (e.g., less than about 40%, less than about 35%, less than about 30%, less than about 25%, or less than about 20%). In some embodiments, the treatment comprises a risk of ARIA-E that is less than about 15% (e.g., less than about 14%, less than about 13%, less than about 12%, less than about 11%, or less than about 10%).

In some embodiments, the treatment results in less than about 45% (e.g., less than about 40%, less than about 35%, less than about 30%, less than about 25%, or less than about 20%) of subjects experiencing symptomatic ARIA-E. In some embodiments, the treatment results less than about 15% (e.g., less than about 14%, less than about 13%, less than about 12%, less than about 11%, or less than about 10%) of subjects experiencing symptomatic ARIA-E. In some embodiments, the treatment results in less than about 10% (e.g., less than about 9%, less than about 8%, less than about 7%, less than about 6%, or less than about 5%) of subjects experiencing symptomatic ARIA-E.

In some embodiments, the risk of ARIA-E is a risk of severe ARIA-E. In some embodiments, the risk of ARIA-E is a risk of moderate+ ARIA-E. In some embodiments, the risk of ARIA-E is a risk of moderate ARIA-E. In some embodiments, the risk of ARIA-E is a risk of mild+ ARIA-E. In some embodiments, the risk of ARIA-E is a risk of mild ARIA-E. In some embodiments, the risk of ARIA-E comprises a risk of FLAIR hyper-intensity at more than one location, wherein each FLAIR location has an extent of 5-10 cm. In some embodiments, the risk of ARIA-E comprises a risk of FLAIR hyper-intensity at one location, wherein the FLAIR location has an extent of 5-10 cm. In some embodiments, the risk of ARIA-E comprises a risk of FLAIR hyper-intensity at more than one location, wherein each FLAIR location has an extent of less than 5 cm, and wherein each FLAIR location is confined to the sulcus, cortex, and/or subcortical white matter. In some embodiments, the risk of ARIA-E comprises a risk of FLAIR hyper-intensity at one location, wherein the FLAIR location has an extent of less than 5 cm, and wherein the FLAIR location is confined to the sulcus, cortex, and/or subcortical white matter.

In some embodiments, the patient is an APOE4 homozygous patient. In some embodiments, the treatment comprises a risk of ARIA-E that is less than about 70% (e.g., less than about 65%, less than about 60%, less than about 55%, or less than about 50%) in the APOE4 homozygous patient. In some embodiments, the treatment comprises a risk of ARIA-E that is less than about 40% (e.g., less than about 35%, less than about 30%, or less than about 25%) in the APOE4 homozygous patient.

In some embodiments, the subject is an APOE4 homozygous subject and the treatment comprises a risk of ARIA-E that is less than about 75% (e.g., less than about 70%, less than about 65%, less than about 60%, less than about 55%, or less than about 50%) in the APOE4 homozygous subject. In some embodiments, the subject is an APOE4 homozygous subject and the treatment comprises a risk of symptomatic ARIA-E that is less than about 30% (e.g., less than about 25%, less than about 20%, or less than about 15%) in the APOE4 homozygous subject

In some embodiments, the patient is an APOE4 heterozygous patient or an APOE4 negative patient. In some embodiments, the treatment comprises a risk of ARIA-E that is less than about 40% (e.g., less than about 35%, less than about 30%, less than about 25%, or less than about 20%) in the APOE4 heterozygous patient or the APOE4 negative patient. In some embodiments, the treatment comprises a risk of ARIA-E that is less than about 15% (e.g., less than about 14%, less than about 13%, less than about 12%, less than about 11%, or less than about 10%) in the APOE4 heterozygous patient or the APOE4 negative patient.

In some embodiments, the subject is an APOE4 heterozygous subject or an APOE4 negative subject and the treatment comprises a risk of ARIA-E that is less than about 45% (e.g., less than about 40%, less than about 35%, less than about 30%, less than about 25%, or less than about 20%) in the APOE4 heterozygous subject or the APOE4 negative subject. In some embodiments, the subject is an APOE4 heterozygous subject or an APOE4 negative subject and the treatment comprises a risk of symptomatic ARIA-E that is less than about 15% (e.g., less than about 14%, less than about 13%, less than about 12%, less than about 11%, or less than about 10%) in the APOE4 heterozygous subject or the APOE4 negative subject. In some embodiments, the risk of ARIA-E is the risk after about 6 months (e.g., about 24 weeks) of treatment. In some embodiments, the risk of ARIA-E is the risk after about 12 months (e.g., about 48 weeks) of treatment. In some embodiments, the risk of ARIA-E is the risk after about 18 months (e.g., about 72 weeks) of treatment.

In some embodiments, the treatment comprises a risk of ARIA-H that is less than about 25% (e.g., less than about 22%, less than about 20%, less than about 18%, or less than about 16%). In some embodiments, the treatment comprises a risk of ARIA-H that is less than about 15% (e.g., less than about 14%, less than about 13%, less than about 12%, less than about 11%, or less than about 10%).

In some embodiments, the risk of ARIA-H is a risk of severe ARIA-H. In some embodiments, the risk of ARIA-H is a risk of moderate ARIA-H. In some embodiments, the risk of ARIA-H is a risk of mild ARIA-H. In some embodiments, the risk of ARIA-H comprises a risk of ≤4 new incidents of microhemorrhages and/or a risk of ≤1 focal area of superficial siderosis. In some embodiments, the risk of ARIA-H comprises a risk of ≤9 new incidents of microhemorrhages and/or a risk of ≤2 focal area of superficial siderosis.

In some embodiments, the risk of ARIA-H is the risk after about 6 months (e.g., about 24 weeks) of treatment. In some embodiments, the risk of ARIA-H is the risk after about 12 months (e.g., about 48 weeks) of treatment. In some embodiments, the risk of ARIA-H is the risk after about 18 months (e.g., about 72 weeks) of treatment.

In some embodiments, the treatment results in less than about 45% (e.g., less than about 40%, less than about 35%, less than about 30%, less than about 25%, or less than about 20%) of subjects experiencing ARIA-E. In some embodiments, the treatment results less than about 15% (e.g., less than about 14%, less than about 13%, less than about 12%, less than about 11%, or less than about 10%) of subjects experiencing ARIA-E. In some embodiments, the treatment results in less than about 10% (e.g., less than about 9%, less than about 8%, less than about 7%, less than about 6%, or less than about 5%) of subjects experiencing ARIA-E.

In some embodiments, the treatment results in less than about 15% (e.g., less than about 14%, less than about 13%, less than about 12%, less than about 11%, less than about 10%, less than about 9%, less than about 8%, less than about 7%, less than about 6%, or less than about 5%) of subjects experiencing symptomatic ARIA-E.

In some embodiments, the treatment results in less than about 25% (e.g., less than about 22%, less than about 20%, less than about 18%, or less than about 16%) of subjects experiencing ARIA-H. In some embodiments, the treatment results in less than about 15% (e.g., less than about 14%, less than about 13%, less than about 12%, less than about 11%, or less than about 10%, less than about 9%, less than about 8%, less than about 7%, less than about 6%, or less than about 5%) of subjects experiencing ARIA-H.

In some embodiments, the ARIA-E and/or ARIA-H levels (e.g., % of subjects experiencing ARIA-E and/or ARIA E) are the ARIA-E and/or ARIA H levels after about 6 months (e.g., about 24 weeks) of treatment. In some embodiments the ARIA-E and/or ARIA-H levels are the ARIA-E and/or ARIA H levels after about 12 months (e.g., about 48 weeks) of treatment. In some embodiments, the ARIA-E and/or ARIA-H are the ARIA-E and/or ARIA H levels after about 18 months (e.g., about 72 weeks) of treatment.

In some embodiments, the patient does not experience symptomatic ARIA, as assessed by Magnetic Resonance Imaging (MRI). In some embodiments, the patient does not experience symptomatic ARIA-E, as assessed by MRI. In some embodiments, the patient does not experience symptomatic ARIA-H, as assessed by MRI. In some embodiments, the patient is an APOE4 heterozygous patient or an APOE4 negative patient. In some embodiments, the patient is an APOE4 homozygous patient.

### Treatment Amenable Patients

The present disclosure is also directed to treatment of Alzheimer's and other amyloidogenic diseases by administration of the antibodies, fragments and pharmaceutical compositions of the disclosure generate a beneficial therapeutic response in a patient (e.g., induction of phagocytosis of Aβ, reduction of plaque burden, inhibition of plaque formation, reduction of neuritic dystrophy, neutralization of soluble, toxic Aβ species, improving cognitive function, and/or reversing, treating or preventing cognitive decline) in the patient, for example, for the prevention or treatment of an amyloidogenic disease. The disclosure is also directed to use of the disclosed antibodies and fragments in the manufacture of a medicament for the treatment or prevention of an amyloidogenic disease.

In one aspect, the disclosure provides methods of preventing or treating a disease associated with amyloid deposits of Aβ in a patient. In one aspect, the amyloid deposits are in the brain or other CNS areas. Such diseases include Alzheimer's disease, Down's syndrome, age-related macular degeneration (AMD), and cognitive impairment. The latter can occur with or without other characteristics of an amyloidogenic disease. Some methods of the disclosure entail administering an effective dosage of an antibody that specifically binds to a component of an amyloid deposit to the patient. Such methods are useful for preventing or treating Alzheimer's disease in human patients

The methods can be used on both asymptomatic patients and those currently showing symptoms of disease. The antibodies used in such methods can be humanized, human or fragments thereof (e.g., antigen binding fragments) and can be monoclonal or polyclonal, as described herein. In yet another aspect, the disclosure features administering antibodies prepared from a human immunized with Aβ peptide, which human can be the patient to be treated with antibody.

In another aspect, the disclosure features administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. Optionally, the polynucleotide encodes heavy and light chains of the antibody. The polynucleotide is expressed to produce the heavy and light chains in the patient. In exemplary embodiments, the patient is monitored for level of administered antibody in the blood of the patient.

Patients amenable to treatment include individuals at risk of disease but not showing symptoms, as well as patients presently showing symptoms. In the case of Alzheimer's disease, potentially anyone who lives long enough is at risk of Alzheimer's disease. Thus, the present methods include administering prophylactically to the general population without the need for any assessment of the risk of the subject patient. The present methods are especially useful for individuals who have a known genetic risk of Alzheimer's disease. Such individuals include those having relatives who have experienced this disease, and those whose risk is determined by analysis of genetic or biochemical markers. Genetic markers of risk toward Alzheimer's disease include mutations in the APP gene, particularly mutations at position 717 and positions 670 and 671 referred to as the Hardy and Swedish mutations, respectively. Other markers of risk are mutations in the presenilin genes, PS1 and PS2, and ApoE4, family history of AD, hypercholesterolemia or atherosclerosis. Individuals presently suffering from Alzheimer's disease can be recognized from characteristic dementia, as well as the presence of risk factors described above. In addition, a number of diagnostic tests are available for identifying individuals who have AD. These include measurement of CSF tau and Aß42 levels. Elevated tau and decreased Aβ42 levels signify the presence of AD. Individuals suffering from Alzheimer's disease can also be diagnosed by ADRDA criteria as discussed in the Examples section.

Treatment in asymptomatic patients can begin at any age (e.g., 10, 20, 30). Usually, however, it is not necessary to begin treatment until a patient reaches 40, 50, 60, or 70. Treatment typically entails multiple dosages over a period of time. Treatment can be monitored by assaying antibody levels over time. If the response falls, a booster dosage is indicated. In the case of potential Down's syndrome patients, treatment can begin antenatally by administering therapeutic agent to the mother or shortly after birth.

### APOE4 Status

Subjects carrying the apolipoprotein E 84 allele (APOE4) are at increased risk of ARIA during treatment with anti-amyloid therapies. See, e.g., C. Dagostin, et al., Efficacy of anti-amyloid-β monoclonal antibody therapy in early Alzheimer's disease: a systematic review and meta-analysis. NEOLOGICAL SCIENCES (2023). Subjects homozygous for APOE4 are at highest risk, due in part to their increased burden of aggregated β amyloid in cerebral microvessels. Furthermore, ongoing research suggests that, during treatment, APOE4 carriers exhibit a larger perivascular Aβ clearance, leading to greater vascular permeability and extravasation of fluid and erythrocytes, thereby resulting higher in ARIA-E and ARIA-H rates. See, e.g., M. Roytman, et al., Amyloid-Related Imaging Abnomaliities: An Update. 220 AM. J. ROENTGENOLOGY 562 (2023).

In some embodiments, the subject is an APOE4 heterozygous subject or an APOE4 negative subject. In some embodiments, the subject is an APOE4 homozygous subject. In some embodiments, the subject is an APOE4 heterozygous subject. In some embodiments, the subject is an APOE4 negative subject (non-carrier).

### In Vivo Detection

In another aspect, the disclosure provides methods for detecting amyloid plaques and deposits in a patient having or at risk of developing an amyloidogenic disease. Such methods are useful for diagnosing or confirming amyloidogenic disease or susceptibility to it. For example, the methods can be used in patients with dementia symptoms, wherein observation of abnormal amyloid deposits likely indicates Alzheimer's disease. The methods can also be used in asymptomatic patients. The presence of abnormal deposits of amyloid indicates susceptibility to future symptomatic disease.

In some embodiments, the method comprises administering to a subject/patient an antibody or fragment thereof of the disclosure and detecting the antibody or fragment thereof bound to Aβ.

Antibody and/or antibody fragments thereof can be administered by any suitable means that results in delivery to the tissue to be visualized, e.g., administered directly into the brain by intravenous injection into the patient's body or by intracranial injection. Dosage of the antibody and/or fragment thereof can comprise a therapeutic dose, subtherapeutic dose or a supratherapeutic dose. In some embodiments the antibody or fragment thereof is labeled, comprising a fluorescent label, a paramagnetic label, or a radioactive label. The choice of label depends on the means of detection. For example, fluorescent labels are suitable for visual detection. The use of paramagnetic labels is suitable for tomographic detection without surgical intervention. In some embodiments, the radioactive label is detected using positron emission tomography (PET) or single-photon emission computed tomography (SPECT).

In another aspect, the disclosure provides methods for measuring the efficacy of treatment in a subject being treated for an amyloidogenic disease. In some embodiments, a first level of amyloid plaque in a subject is measured prior to treatment by administering an antibody or fragment thereof of the disclosure and detecting a first amount of the antibody or fragment thereof bound to Aβ in the subject. A treatment can then be administered to the subject, followed by measuring a second level of amyloid plaque in the subject, and detecting the antibody or fragment thereof bound to Aβ in the subject. In some embodiments, a decrease in the level of amyloid plaque indicates a positive response to treatment, and in some embodiments, no change in the level of amyloid plaque or a small increase in amyloid plaque indicates a positive response to treatment. In some embodiments, levels of amyloid plaque can be measured utilizing the methods of detecting amyloid plaques described herein.

In some embodiments, diagnosis of an amyloidogenic disease can be performed, for example, by comparing the number, size and/or intensity of labeled positions from a measured first level (i.e., baseline) to a subsequent second level of amyloid plaque in a subject. An increase over time indicates disease progression, no change indicates, and fewer or less intense amyloid plaques over time indicates remission.

Detection of brain amyloid plaques is conducted by methods known to one of skill in the art. In some embodiments, amyloid plaque burden is measured in a patient by positron emission tomography (PET) imaging. PET imaging agents are known to one of skill in the art and include 18F-florbetapir, florbetaben F18, and flutemetamol F18. In some embodiments, amyloid plaque, as measured by PET, is quantified by a composite standard uptake value ratio (SUVR). In some embodiments, amyloid plaque, as measured by PET, is calculated using the Centiloid scale. In some embodiments, change in amyloid plaque burden is measured by change in SUVR over time. In some embodiments, change in amyloid plaque burden is measured by change in Centiloid over time. See, Navitsky M, Joshi A D, Kennedy I, et al., Standardization of amyloid quantitation with florbetapir standardized uptake value ratios to the Centiloid scale, Alzheimers Dement 2018 14:1565-71 and Oshi A D, Pontecorvo M J, Lu M, et al., A Semiautomated Method for Quantification of F 18 Florbetapir PET Images, J Nucl Med. 2015; 56 (11): 1736-41.

### Uses

In various aspects, the disclosure is directed to pharmaceutical compositions comprising the anti-amyloid β antibody or an antigen-binding fragment as described herein for treating Alzheimer's disease in a subject. In the various aspects, the treatment includes administering to the subject about 20 mg to about 200 mg of the antibody or antigen binding fragment thereof once about every 3-5 weeks. In aspects, the intermediate doses as described herein can be used for subcutaneous administration.

In various aspects, the disclosure is directed to pharmaceutical compositions comprising an anti-amyloid β antibody or an antigen-binding fragment for reducing amyloid plaque in a subject. In the various aspects, the treatment of the subject includes administering to the subject about 20 mg to about 200 mg of an anti-amyloid β antibody or the antigen-binding fragment thereof once about every 3-5 weeks. In aspects, the intermediate doses as described herein can be used for subcutaneous administration.

In various aspects, the disclosure is directed to pharmaceutical compositions comprising an anti-amyloid β antibody or an antigen-binding fragment as described herein for converting a subject from amyloid positive to amyloid negative. In the various aspects, treatment of the subject includes administering to the subject about 20 mg to about 200 mg of the anti-amyloid β antibody or the antigen-binding fragment thereof once about every 3-5 weeks. In aspects, the intermediate doses as described herein can be used for subcutaneous administration.

In embodiments of various pharmaceutical compositions, the administration includes, for example, subcutaneously administering to the subject about 45 mg of an anti-amyloid β antibody once about every 4 weeks, subcutaneously administering to the subject about 70 mg of an anti-amyloid β antibody once about every 4 weeks, or subcutaneously administering to the subject about 200 mg of an anti-amyloid β antibody once about every 4 weeks. In embodiments, the pharmaceutical compositions include pharmaceutically acceptable excipients for administration, including, for example, subcutaneous administration.

In various aspects, the disclosure is directed to the use of an anti-amyloid antibody or an antigen-binding fragment as described herein for the manufacture of a medicament for treating Alzheimer's disease in a subject. In various aspects, the medicament is for administration to the subject at about 20 mg to about 200 mg of the anti-amyloid β antibody or an antigen-binding fragment once about every 3-5 weeks. In aspects, the intermediate doses as described herein can be used for subcutaneous administration.

In various aspects, the disclosure is directed to the use of an anti-amyloid β antibody or an antigen-binding fragment as described herein for the manufacture of a medicament for reducing amyloid plaque in a subject. In various aspects, the medicament is for administration to the subject about 20 mg to about 200 mg of the anti-amyloid β antibody or the antigen-binding fragment thereof once about every 3-5 weeks. In aspects, the intermediate doses as described herein can be used for subcutaneous administration.

In various aspects, the disclosure is directed to the use of an anti-amyloid β antibody or an antigen-binding fragment as described herein for the manufacture of a medicament for converting a subject from amyloid positive to amyloid negative. In the various aspects, the medicament is for administration to the subject to the subject at about 20 mg to about 200 mg of the anti-amyloid β antibody or the antigen-binding fragment thereof once about every 3-5 weeks. In aspects, the intermediate doses as described herein can be used for subcutaneous administration.

In embodiments of the uses of the anti-amyloid β antibody or the antigen-binding fragment thereof, the administration includes, for example, subcutaneously administering to the subject about 45 mg of an anti-amyloid β antibody or binding fragment once about every 4 weeks, subcutaneously administering to the subject about 70 mg of an anti-amyloid β antibody or binding fragment once about every 4 weeks, or subcutaneously administering to the subject about 200 mg of an anti-amyloid β antibody or binding fragment once about every 4 weeks.

The present disclosure will be more fully described by the following non-limiting examples.

SEQ ID NO: 40: huIgG1 Constant
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKENWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 41: huKappa Constant
RTVAAPSVFIFPPSDEQLKSGTASVVCLLNNFY PREAKVQWKVDNALQSGNSQESVTEQDSK
DSTYSLSSTLTLSKADYEKHKVYACEVTHQGLSSPVTKSENRGEC
SEQ ID NO: 42: h2726_VH (Variable Heavy) Nucleotide Sequence
GAAGTGCAGCTTCTGGAGAGCGGGGGCGGCCTGGTGCAGCCGGGCGGATCCCTGAGACTGTC
CTGTGCCGCGTCCGGTTTTACCTTCTCCAACTACGGAATGTCATGGGTCCGCCAAGCACCCG
GAAAGGGATTGGAATGGGTGGCTTCGATCCGGTCCGGCTCGGGACGGACCTACTACTCCGAT
AACGTCAAGGGCAGATTCACTATTAGCCGGGACAACAGCAAGAATACCCTGTACCTCCAAAT
GAACTCCCTGAGGGCCGAGGACACCGCCGTGTATTACTGCGTGCGCTACGACCACTACTCGG
GTTCCTCTGATTACTGGGGACAGGGGACCCTCGTGACTGTGTCAAGC
SEQ ID NO: 43: h2726_VL (Variable Light) Nucleotide Sequence
GATGTCGTGATGACCCAGTCACCACTGTCCCTTCCTGTGACTCCCGGAGAACCGGCGTCCAT
TTCGTGCAAGAGCAGCCAGTCCCTGCTCGATTATGACGGAAAGACCTACCTGAACTGGTTGC
TCCAAAAGCCTGGGCAGAGCCCCCAGAGACTGATCTACAAAGTGTCCAACAGGGACTCGGGC
GTGCCGGACCGCTTCTCGGGGTCCGGTTCCGGTACCGACTTTACGCTGAAGATCTCACGGGT
GGAAGCCGAGGACGTGGGAGTGTACTACTGTTGGCAGGGCACTCACTTCCCGCGGACCTTCG
GACAAGGCACCAAGGTCGAGATCAAG
SEQ ID NO: 44: h2931_VH (Variable Heavy) Nucleotide Sequence
GAAGTGCAGCTCCTGGAGTCCGGGGGTGGACTGGTGCAGCCCGGGGGCAGCCTGAGGCTGAG
CTGCGCCGCGTCAGGATTCACCTTCTCCAACTTCGGAATGTCCTGGGTCAGACAGGCCCCGG
GAAAGGGCCTTGAATGGGTGGCTAGCGTGCGCTCCGGTTCCGGACGGACCTACTACTCGGAC
AACGTGAAGGGCCGGTTTACTATCTCCCGGGACAATTCGAAGAACACCCTGTACCTCCAAAT
GAACTCCTTGCGCGCCGAGGATACCGCAGTGTATTACTGCGTGCGCTACGACCACTACTCTG
GCACTAGCGATTACTGGGGCCAGGGAACTCTGGTCACCGTGTCGTCA
SEQ ID NO: 45: h2931_VL (Variable Light) Nucleotide Sequence
GATGTCGTGATGACTCAGTCACCTCTGTCCCTGCCTGTGACCCTTGGGGAACCCGCCTCGAT
CTCGTGCAAGAGCTCCCAGAGCCTGCTCGACTATGATGGAAAGACCTACCTGAACTGGTTGC
TCCAAAAGCCGGGCCAGAGCCCCCAGAGGCTGATCTACCGCGTGACCAACCGCGACACCGGG
GTGCCGGACCGGTTCTCCGGATCCGGCAGCGGCACTGACTTCACCCTGAAAATTTCCAGAGT
GGAAGCCGAGGACGTGGGAGTGTACTACTGTTGGCAGGGTACTCACTTTCCACGGTCCTTCG
GTCAAGGAACCAAGGTCGAGATCAAG
SEQ ID NO: 46: h2731_VH (Variable Heavy) Nucleotide Sequence
GAAGTGCAGCTTCTGGAGAGCGGGGGCGGCCTGGTGCAGCCGGGCGGATCCCTGAGACTGTC
CTGTGCCGCGTCCGGTTTTACCTTCTCCAACTACGGAATGTCATGGGTCCGCCAAGCACCCG
GAAAGGGATTGGAATGGGTGGCTTCGATCCGGTCCGGCTCGGGACGGACCTACTACTCCGAT
AACGTCAAGGGCAGATTCACTATTAGCCGGGACAACAGCAAGAATACCCTGTACCTCCAAAT
GAACTCCCTGAGGGCCGAGGACACCGCCGTGTATTACTGCGTGCGCTACGACCACTACTCGG
GTTCCTCTGATTACTGGGGACAGGGGACCCTCGTGACTGTGTCAAGC
SEQ ID NO: 47: h2731_VL (Variable Light) Nucleotide Sequence
GATGTCGTGATGACTCAGTCACCTCTGTCCCTGCCTGTGACCCTTGGGGAACCCGCCTCGAT
CTCGTGCAAGAGCTCCCAGAGCCTGCTCGACTATGATGGAAAGACCTACCTGAACTGGTTGC
TCCAAAAGCCGGGCCAGAGCCCCCAGAGGCTGATCTACCGCGTGACCAACCGCGACACCGGG
GTGCCGGACCGGTTCTCCGGATCCGGCAGCGGCACTGACTTCACCCTGAAAATTTCCAGAGT
GGAAGCCGAGGACGTGGGAGTGTACTACTGTTGGCAGGGTACTCACTTTCCACGGTCCTTCG
GTCAAGGAACCAAGGTCGAGATCAAG
SEQ ID NO: 48: h2831_VH (Variable Heavy) Nucleotide Sequence
GAAGTGCAGCTGCTGGAGTCTGGCGGCGGACTGGTGCAGCCCGGGGGATCCCTGCGGCTTTC
CTGCGCCGCATCCGGCTTCACCTTTTCAAACTTCGGAATGTCGTGGGTCAGACAGGCCCCGG
GAAAGGGTCTGGAATGGGTGGCCTCAGTGCGGTCCGGATCGGGTAGAACCTACTACAGCGAT
AACGTGAAGGGCCGGTTCACGATCTCCCGCGACAACTCCAAGAACACCCTGTACTTGCAAAT
GAATAGCCTCAGGGCTGAGGATACCGCGGTCTACTACTGTGTGCGCTATGACCACTACACTG
GAACTAGCGACTACTGGGGCCAGGGGACCCTCGTGACTGTGTCGTCC
SEQ ID NO: 49: h2831_VL (Variable Light) Nucleotide Sequence
GATGTCGTGATGACTCAGTCACCTCTGTCCCTGCCTGTGACCCTTGGGGAACCCGCCTCGAT
CTCGTGCAAGAGCTCCCAGAGCCTGCTCGACTATGATGGAAAGACCTACCTGAACTGGTTGC
TCCAAAAGCCGGGCCAGAGCCCCCAGAGGCTGATCTACCGCGTGACCAACCGCGACACCGGG
GTGCCGGACCGGTTCTCCGGATCCGGCAGCGGCACTGACTTCACCCTGAAAATTTCCAGAGT
GGAAGCCGAGGACGTGGGAGTGTACTACTGTTGGCAGGGTACTCACTTTCCACGGTCCTTCG
GTCAAGGAACCAAGGTCGAGATCAAG
SEQ ID NO: 50: h2926_VH (Variable Heavy) Nucleotide Sequence
GAAGTGCAGCTCCTGGAGTCCGGGGGTGGACTGGTGCAGCCCGGGGGCAGCCTGAGGCTGAG
CTGCGCCGCGTCAGGATTCACCTTCTCCAACTTCGGAATGTCCTGGGTCAGACAGGCCCCGG
GAAAGGGCCTTGAATGGGTGGCTAGCGTGCGCTCCGGTTCCGGACGGACCTACTACTCGGAC
AACGTGAAGGGCCGGTTTACTATCTCCCGGGACAATTCGAAGAACACCCTGTACCTCCAAAT
GAACTCCTTGCGCGCCGAGGATACCGCAGTGTATTACTGCGTGCGCTACGACCACTACTCTG
GCACTAGCGATTACTGGGGCCAGGGAACTCTGGTCACCGTGTCGTCA
SEQ ID NO: 51: h2926_VL (Variable Light) Nucleotide Sequence
GATGTCGTGATGACCCAGTCACCACTGTCCCTTCCTGTGACTCCCGGAGAACCGGCGTCCAT
TTCGTGCAAGAGCAGCCAGTCCCTGCTCGATTATGACGGAAAGACCTACCTGAACTGGTTGC
TCCAAAAGCCTGGGCAGAGCCCCCAGAGACTGATCTACAAAGTGTCCAACAGGGACTCGGGC
GTGCCGGACCGCTTCTCGGGGTCCGGTTCCGGTACCGACTTTACGCTGAAGATCTCACGGGT
GGAAGCCGAGGACGTGGGAGTGTACTACTGTTGGCAGGGCACTCACTTCCCGCGGACCTTCG
GACAAGGCACCAAGGTCGAGATCAAG
SEQ ID NO: 52: h4921G VH (Variable Heavy) Nucleotide Sequence
GAGGTGCAGCTGCTGGAGTCGGGGGGGGGACTCGTGCAGCCCGGGGGCTCCCTGAGACTCTC
TTGTGCCGCCTCCGGCTTCACTTTTTCAAACTTCGGAATGTCCTGGGTCCGCCAAGCACCGG
GAAAGGGTCTGGAATGGGTCGCCAGCGTGCGGTCCGGCGGCGGACGGACTTACTACTCCGAC
AACGTGAAGGGCCGGTTCACCATCTCAAGGGATAACTCCAAGAATACTCTGTACTTGCAAAT
GAACTCGCTGCGCGCTGAAGATACCGCGGTGTACTATTGCGTGCGCTACGACCACTACTCCG
GTACCAGCGACTACTGGGGACAGGGAACCCTTGTGACCGTGTCGAGC
SEQ ID NO: 53: h4921G VL (Variable Light) Nucleotide Sequence
GATGTCGTGATGACTCAGTCGCCCCTCTCCCTGCCTGTGACTCTGGGGGAACCCGCGTCCAT
TTCGTGCAAGAGCAGCCAGTCCCTGTTGGACTCAGACGGAAAGACCTACCTTAACTGGCTGC
TGCAAAAGCCAGGACAGAGCCCGCAGAGGCTGATCTACCGCGTGACCAACCGGGATACGGGA
GTGCCGGACAGATTCAGCGGCTCGGGTTCCGGCACCGACTTCACCCTCAAAATCTCCCGCGT
CGAGGCCGAGGACGTGGGCGTGTATTACTGTTGGCAGGGAACCCACTTTCCTCGGACCTTCG
GTCAAGGGACTAAGGTCGAAATCAAG
SEQ ID NO: 54: h2826 VH (Variable Heavy) Nucleotide Sequence
GAAGTGCAGCTGCTGGAGTCTGGCGGCGGACTGGTGCAGCCCGGGGGATCCCTGCGGCTTTC
CTGCGCCGCATCCGGCTTCACCTTTTCAAACTTCGGAATGTCGTGGGTCAGACAGGCCCCGG
GAAAGGGTCTGGAATGGGTGGCCTCAGTGCGGTCCGGATCGGGTAGAACCTACTACAGCGAT
AACGTGAAGGGCCGGTTCACGATCTCCCGCGACAACTCCAAGAACACCCTGTACTTGCAAAT
GAATAGCCTCAGGGCTGAGGATACCGCGGTCTACTACTGTGTGCGCTATGACCACTACACTG
GAACTAGCGACTACTGGGGCCAGGGGACCCTCGTGACTGTGTCGTCC
SEQ ID NO: 55: h2826 VL (Variable Light) Nucleotide Sequence
GATGTCGTGATGACCCAGTCACCACTGTCCCTTCCTGTGACTCCCGGAGAACCGGCGTCCAT
TTCGTGCAAGAGCAGCCAGTCCCTGCTCGATTATGACGGAAAGACCTACCTGAACTGGTTGC
TCCAAAAGCCTGGGCAGAGCCCCCAGAGACTGATCTACAAAGTGTCCAACAGGGACTCGGGC
GTGCCGGACCGCTTCTCGGGGTCCGGTTCCGGTACCGACTTTACGCTGAAGATCTCACGGGT
GGAAGCCGAGGACGTGGGAGTGTACTACTGTTGGCAGGGCACTCACTTCCCGCGGACCTTCG
GACAAGGCACCAAGGTCGAGATCAAG
SEQ ID NO: 56: h2929 VH (Variable Heavy) Nucleotide Sequence
GAAGTGCAGCTCCTGGAGTCCGGGGGTGGACTGGTGCAGCCCGGGGGCAGCCTGAGGCTGAG
CTGCGCCGCGTCAGGATTCACCTTCTCCAACTTCGGAATGTCCTGGGTCAGACAGGCCCCGG
GAAAGGGCCTTGAATGGGTGGCTAGCGTGCGCTCCGGTTCCGGACGGACCTACTACTCGGAC
AACGTGAAGGGCCGGTTTACTATCTCCCGGGACAATTCGAAGAACACCCTGTACCTCCAAAT
GAACTCCTTGCGCGCCGAGGATACCGCAGTGTATTACTGCGTGCGCTACGACCACTACTCTG
GCACTAGCGATTACTGGGGCCAGGGAACTCTGGTCACCGTGTCGTCA
SEQ ID NO: 57: h2929 VL (Variable Light) Nucleotide Sequence
GATGTCGTGATGACCCAAAGCCCCCTGTCCCTCCCTGTGACTCCTGGAGAGCCGGCGTCCAT
TTCCTGCCGGTCAAGCCAGTCCTTGGTGGACTACGACGGAAAGACCTACCTCAACTGGCTGC
TGCAGCGCCCCGGGCAGTCGCCGCAGCGGCTTATCTACAAAGTGTCCAACCGCGACTCGGGC
GTGCCGGATAGGTTTTCGGGTTCCGGAAGCGGCACCGACTTCACCCTGAAAATCTCCAGAGT
GGAAGCCGAGGACGTGGGAGTGTACTACTGTTGGCAGGGTTCTCACTTCCCACGGTCATATG
GCCAAGGGACTAAGGTCGAAATCAAG
SEQ ID NO: 58: h3818G VH (Variable Heavy) Nucleotide Sequence
GAAGTGCAGCTCCTGGAGTCCGGCGGTGGACTGGTGCAGCCGGGCGGATCCCTGAGACTGTC
CTGCGCCGCGTCGGGCTTTACTTTCGCAAATTACGGCATGAGCTGGGTCAGACAGGCCCCCG
GGAAGGGTCTGGAATGGGTGGCCAGCGTCCGGAGCGGGGGATCCCGGACCTATTACTCCGAC
AACGTGAAGGGCCGCTTCACCATCTCAAGGGACAACTCCAAGAACACCCTGTACTTGCAAAT
GAACAGCCTTCGGGCTGAGGATACTGCCGTGTACTACTGCGTGCGCTACGACCACTACTCCG
GATCCTCGGATTACTGGGGACAGGGAACCCTCGTGACCGTGTCATCG
SEQ ID NO: 59: h3818G VL (Variable Light) Nucleotide Sequence
GATGTCGTGATGACTCAGTCGCCCCTCTCCCTGCCTGTGACTCTGGGGGAACCCGCGTCCAT
TTCGTGCAAGAGCAGCCAGTCCCTGATGGACACCGACGGAAAGACCTACCTTAACTGGCTGC
TGCAAAAGCCAGGACAGAGCCCGCAGAGGCTGATCTACAAAGTGTCAAACCGGGAGTCCGGA
GTGCCGGACAGATTCAGCGGCTCGGGTTCCGGCACCGACTTCACCCTCAAAATCTCCCGCGT
CGAGGCCGAGGACGTGGGCGTGTATTACTGTTGGCAGGGAACCCACTTTCCTCGGACCTTCG
GTCAAGGGACTAAGGTCGAAATCAAG
SEQ ID NO: 60: h2927 VH (Variable Heavy) Nucleotide Sequence
GAAGTGCAGCTCCTGGAGTCCGGGGGTGGACTGGTGCAGCCCGGGGGCAGCCTGAGGCTGAG
CTGCGCCGCGTCAGGATTCACCTTCTCCAACTTCGGAATGTCCTGGGTCAGACAGGCCCCGG
GAAAGGGCCTTGAATGGGTGGCTAGCGTGCGCTCCGGTTCCGGACGGACCTACTACTCGGAC
AACGTGAAGGGCCGGTTTACTATCTCCCGGGACAATTCGAAGAACACCCTGTACCTCCAAAT
GAACTCCTTGCGCGCCGAGGATACCGCAGTGTATTACTGCGTGCGCTACGACCACTACTCTG
GCACTAGCGATTACTGGGGCCAGGGAACTCTGGTCACCGTGTCGTCA
SEQ ID NO: 61: h2927 VL (Variable Light) Nucleotide Sequence
GATGTCGTGATGACTCAGTCACCGCTCTCCCTCCCTGTGACCCCGGGCGAACCAGCGTCGAT
CTCCTGCAAGAGCAGCCAATCATTGCTGGACTACGACGGAAAGACCTATCTTAACTGGCTGC
TGCAGAAGCCCGGGCAGAGCCCGCAGCGCCTGATCTACAAAGTGTCCAACAGAGACTCCGGA
GTGCCTGATAGGTTCTCGGGTTCCGGCTCCGGTACCGACTTCACTCTGAAAATTTCCCGGGT
GGAAGCCGAGGACGTGGGAGTGTACTACTGTTGGCAGGGCACCCACTTCCCCCGGTCGTTTG
GACAAGGGACCAAGGTCGAGATCAAG
SEQ ID NO: 62: h49K3G VH (Variable Heavy) Nucleotide Sequence
GAGGTGCAGCTGCTGGAGTCGGGGGGGGGACTCGTGCAGCCCGGGGGCTCCCTGAGACTCTC
TTGTGCCGCCTCCGGCTTCACTTTTTCAAACTTCGGAATGTCCTGGGTCCGCCAAGCACCGG
GAAAGGGTCTGGAATGGGTCGCCAGCGTGCGGTCCGGCGGCGGACGGACTTACTACTCCGAC
AACGTGAAGGGCCGGTTCACCATCTCAAGGGATAACTCCAAGAATACTCTGTACTTGCAAAT
GAACTCGCTGCGCGCTGAAGATACCGCGGTGTACTATTGCGTGCGCTACGACCACTACTCCG
GTACCAGCGACTACTGGGGACAGGGAACCCTTGTGACCGTGTCGAGC
SEQ ID NO: 63: h49K3G VL (Variable Light) Nucleotide Sequence
GATGTCGTGATGACTCAGTCGCCCCTCTCCCTGCCTGTGACTCTGGGGGAACCCGCGTCCAT
TTCGTGCAAGAGCAGCCAGTCCCTGTTGGACTCAGACGGAAAGACCTACCTTAACTGGCTGC
TGCAAAAGCCAGGACAGAGCCCGCAGAGGCTGATCTACAAAGTGTCAAACCGGGATTCCGGA
GTGCCGGACAGATTCAGCGGCTCGGGTTCCGGCACCGACTTCACCCTCAAAATCTCCCGCGT
CGAGGCCGAGGACGTGGGCGTGTATTACTGTTGGCAGGGAACCCACTTTCCTCGGACCTTCG
GTCAAGGGACTAAGGTCGAAATCAAG
SEQ ID NO: 64: h4917G VH (Variable Heavy) Nucleotide Sequence
GAGGTGCAGCTGCTGGAGTCGGGGGGGGGACTCGTGCAGCCCGGGGGCTCCCTGAGACTCTC
TTGTGCCGCCTCCGGCTTCACTTTTTCAAACTTCGGAATGTCCTGGGTCCGCCAAGCACCGG
GAAAGGGTCTGGAATGGGTCGCCAGCGTGCGGTCCGGCGGCGGACGGACTTACTACTCCGAC
AACGTGAAGGGCCGGTTCACCATCTCAAGGGATAACTCCAAGAATACTCTGTACTTGCAAAT
GAACTCGCTGCGCGCTGAAGATACCGCGGTGTACTATTGCGTGCGCTACGACCACTACTCCG
GTACCAGCGACTACTGGGGACAGGGAACCCTTGTGACCGTGTCGAGC
SEQ ID NO: 65: h4917G VL (Variable Light) Nucleotide Sequence
GATGTCGTGATGACTCAGTCGCCCCTCTCCCTGCCTGTGACTCTGGGGGAACCCGCGTCCAT
TTCGTGCAAGAGCAGCCAGTCCCTGTTGGACTCAGACGGAAAGACCTACCTTAACTGGCTGC
TGCAAAAGCCAGGACAGAGCCCGCAGAGGCTGATCTACAAAGTGACCAACCGGGAGTCCGGA
GTGCCGGACAGATTCAGCGGCTCGGGTTCCGGCACCGACTTCACCCTCAAAATCTCCCGCGT
CGAGGCCGAGGACGTGGGCGTGTATTACTGTTGGCAGGGAACCCACTTTCCTCGGTCATTCG
GTCAAGGGACTAAGGTCGAAATCAAG
SEQ ID NO: 66: h2727 VH (Variable Heavy) Nucleotide Sequence
GAAGTGCAGCTTCTGGAGAGCGGGGGCGGCCTGGTGCAGCCGGGCGGATCCCTGAGACTGTC
CTGTGCCGCGTCCGGTTTTACCTTCTCCAACTACGGAATGTCATGGGTCCGCCAAGCACCCG
GAAAGGGATTGGAATGGGTGGCTTCGATCCGGTCCGGCTCGGGACGGACCTACTACTCCGAT
AACGTCAAGGGCAGATTCACTATTAGCCGGGACAACAGCAAGAATACCCTGTACCTCCAAAT
GAACTCCCTGAGGGCCGAGGACACCGCCGTGTATTACTGCGTGCGCTACGACCACTACTCGG
GTTCCTCTGATTACTGGGGACAGGGGACCCTCGTGACTGTGTCAAGC
SEQ ID NO: 67: h2727 VL (Variable Light) Nucleotide Sequence
GATGTCGTGATGACTCAGTCACCGCTCTCCCTCCCTGTGACCCCGGGCGAACCAGCGTCGAT
CTCCTGCAAGAGCAGCCAATCATTGCTGGACTACGACGGAAAGACCTATCTTAACTGGCTGC
TGCAGAAGCCCGGGCAGAGCCCGCAGCGCCTGATCTACAAAGTGTCCAACAGAGACTCCGGA
GTGCCTGATAGGTTCTCGGGTTCCGGCTCCGGTACCGACTTCACTCTGAAAATTTCCCGGGT
GGAAGCCGAGGACGTGGGAGTGTACTACTGTTGGCAGGGCACCCACTTCCCCCGGTCGTTTG
GACAAGGGACCAAGGTCGAGATCAAG
SEQ ID NO: 68: h4918G VH (Variable Heavy) Nucleotide Sequence
GAGGTGCAGCTGCTGGAGTCGGGGGGGGGACTCGTGCAGCCCGGGGGCTCCCTGAGACTCTC
TTGTGCCGCCTCCGGCTTCACTTTTTCAAACTTCGGAATGTCCTGGGTCCGCCAAGCACCGG
GAAAGGGTCTGGAATGGGTCGCCAGCGTGCGGTCCGGCGGCGGACGGACTTACTACTCCGAC
AACGTGAAGGGCCGGTTCACCATCTCAAGGGATAACTCCAAGAATACTCTGTACTTGCAAAT
GAACTCGCTGCGCGCTGAAGATACCGCGGTGTACTATTGCGTGCGCTACGACCACTACTCCG
GTACCAGCGACTACTGGGGACAGGGAACCCTTGTGACCGTGTCGAGC
SEQ ID NO: 69: h4918G VL (Variable Light) Nucleotide Sequence
GATGTCGTGATGACTCAGTCGCCCCTCTCCCTGCCTGTGACTCTGGGGGAACCCGCGTCCAT
TTCGTGCAAGAGCAGCCAGTCCCTGATGGACACCGACGGAAAGACCTACCTTAACTGGCTGC
TGCAAAAGCCAGGACAGAGCCCGCAGAGGCTGATCTACAAAGTGTCAAACCGGGAGTCCGGA
GTGCCGGACAGATTCAGCGGCTCGGGTTCCGGCACCGACTTCACCCTCAAAATCTCCCGCGT
CGAGGCCGAGGACGTGGGCGTGTATTACTGTTGGCAGGGAACCCACTTTCCTCGGACCTTCG
GTCAAGGGACTAAGGTCGAAATCAAG
SEQ ID NO: 70: Aducanumab Heavy Chain:
QVQLVESGGGVVQPGRSLRLSCAASGFAFSSYGMHWVRQAPGKGLEWVAVIWEDGTKKYYTD
SVKGRFTISRDNSKNTLYLQMNTLRAEDTAVYYCARDRGIGARRGPYYMDVWGKGTTVTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKRVEPKSCDKTHTCPPCPAPELLGGPSVF
LFPPKPKDTLMISRTPEVTCVVVDVSHEDPEVKENWYVDGVEVHNAKTKPREEQYNSTYRVV
SVLTVLHQDWLNGKEYKCKVSNKALPAPIEKTISKAKGQPREPQVYTLPPSREEMTKNQVSL
TCLVKGFYPSDIAVEWESNGQPENNYKTTPPVLDSDGSFFLYSKLTVDKSRWQQGNVESCSV
MHEALHNHYTQKSLSLSPGK
SEQ ID NO: 71: Aducanumab Light Chain:
DIQMTQSPSSLSASVGDRVTITCRASQSISSYLNWYQQKPGKAPKLLIYAASSLQSGVPSRF
SGSGSGTDFTLTISSLQPEDFATYYCQQSYSTPLTFGGGTKVEIKRTVAAPSVFIFPPSDEQ
LKSGTASVVCLLNNFYPREAKVQWKVDNALQSGNSQESVTEQDSKDSTYSLSSTLTLSKADY
EKHKVYACEVTHQGLSSPVTKSENRGEC
SEQ ID NO: 72: Bapineuzumab HC (Heavy Chain)
EVQLLESGGGLVQPGGSLRLSCAASGFTFSNYGMSWVRQAPGKGLEWVASIRSGGGRTYYSD
NVKGRFTISRDNSKNTLYLQMNSLRAEDTAVYYCVRYDHYSGSSDYWGQGTLVTVSSASTKG
PSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPK
PKDTLMISRTPEVTCVVVDVSHEDPEVKENWYVDGVEVHNAKTKPREEQYNSTYRVVSVLTV
LHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEAL
HNHYTQKSLSLSPGK
SEQ ID NO: 73: Bapineuzumab VH (Variable Heavy)
EVQLLESGGGLVQPGGSLRLSCAASGFTFSNYGMSWVRQAPGKGLEWVASIRSGGGRTYYSD
NVKGRFTISRDNSKNTLYLQMNSLRAEDTAVYYCVRYDHYSGSSDYWGQGTLVTVSS
SEQ ID NO: 16: VH CDR1
GFTFSNYGMS
SEQ ID NO: 17: VH CDR2
SIRSGGGRTYYSNDYNVKG
SEQ ID NO: 18: VH CDR3
YDHYSGSSDY
SEQ ID NO: 77: Bapineuzumab LC (Light Chain)
DVVMTQSPLSLPVTPGEPASISCKSSQSLLDSDGKTYLNWLLQKPGQSPQRLIYLVSKLDSG
VPDRFSGSGSGTDFTLKISRVEAEDVGVYYCWQGTHEPRTFGQGTKVEIKRTVAAPSVFIFP
PSDEQLKSGTASVVCLLNNFYPREAKVQWKVDNALQSGNSQESVTEQDSKDSTYSLSSTLTL
SKADYEKHKVYACEVTHQGLSSPVTKSENRGEC
SEQ ID NO: 78: Bapineuzumab VL (Variable Light)
DVVMTQSPLSLPVTPGEPASISCKSSQSLLDSDGKTYLNWLLQKPGQSPQRLIYLVSKLDSG
VPDRFSGSGSGTDFTLKISRVEAEDVGVYYCWQGTHEPRTFGQGTKVEIK
SEQ ID NO: 26: VL CDR1
KSSQSLLDSDGKTYLN
SEQ ID NO: 27: VL CDR2
LVSKLDS
SEQ ID NO: 28: VL CDR3
WQGTHFPRT
SEQ ID NO: 82: Gantenerumab HC amino acid sequence:
QVELVESGGGLVQPGGSLRLSCAASGFTFSSYAMSWVRQAPGKGLEWVSAINASGTRTYYAD
SVKGRFTISRDNSKNTLYLQMNSLRAEDTAVYYCARGKGNTHKPYGYVRYFDVWGQGTLVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVDKSRWQQGNVFSC
SVMHEALHNHYTQKSLSLSPGK
SEQ ID NO 83: Gantenerumab LC amino acid sequence:
DIVLTQSPATLSLSPGERATLSCRASQSVSSSYLAWYQQKPGQAPRLLIYGASSRATGVPAR
FSGSGSGTDFTLTISSLEPEDFATYYCLQIYNMPITFGQGTKVEIKRTVAAPSVFIFPPSDE
QLKSGTASVVCLLNNFYPREAKVQWKVDNALQSGNSQESVTEQDSKDSTYSLSSTLTLSKAD
YEKHKVYACEVTHQGLSSPVTKS ENRGEC
SEQ ID NO: 84: Amyloid Beta (Aβ) 1-42:
DAEFRHDSGYEVHHQKLVFFAEDVGSNKGAIIGLMVGGVVIA
SEQ ID NO: 85: Amyloid Beta (Aβ) Precursor Protein:
MLPGLALLLLAAWTARALEVPTDGNAGLLAEPQIAMFCGRLNMHMNVQNGKWDSDPSGTKTC
IDTKEGILQYCQEVYPELQITNVVEANQPVTIQNWCKRGRKQCKTHPHFVIPYRCLVGEFVS
DALLVPDKCKFLHQERMDVCETHLHWHTVAKETCSEKSTNLHDYGMLLPCGIDKFRGVEFVC
CPLAEESDNVDSADAEEDDSDVWWGGADTDYADGSEDKVVEVAEEEEVAEVEEEEADDDEDD
EDGDEVEEEAEEPYEEATERTTSIATTTTTTTESVEEVVREVCSEQAETGPCRAMISRWYFD
VTEGKCAPFFYGGCGGNRNNFDTEEYCMAVCGSAMSQSLLKTTQEPLARDPVKLPTTAASTP
DAVDKYLETPGDENEHAHFQKAKERLEAKHRERMSQVMREWEEAERQAKNLPKADKKAVIQH
FQEKVESLEQEAANERQQLVETHMARVEAMLNDRRRLALENYITALQAVPPRPRHVENMLKK
YVRAEQKDRQHTLKHFEHVRMVDPKKAAQIRSQVMTHLRVIYERMNQSLSLLYNVPAVAEEI
QDEVDELLQKEQNYSDDVLANMISEPRISYGNDALMPSLTETKTTVELLPVNGEFSLDDLQP
WHSFGADSVPANTENEVEPVDARPAADRGLTTRPGSGLTNIKTEEISEVKMDAEFRHDSGYE
VHHQKLVFFAEDVGSNKGAIIGLMVGGVVIATVIVITLVMLKKKQYTSIHHGVVEVDAAVTP
EERHLSKMQQNGYENPTYKFFEQMQN
SEQ ID NO: 86: huIgG1 Constant Nucleotide Sequence
GCCAGCACTAAGGGGCCTAGCGTCTTTCCGCTGGCCCCGTCCTCCAAGTCCACTTCGGGTGG
AACCGCGGCACTGGGGTGCCTCGTGAAGGACTACTTCCCCGAGCCGGTCACCGTGTCCTGGA
ACTCGGGAGCCCTGACCTCCGGAGTGCATACTTTCCCTGCGGTGCTGCAGTCCTCCGGGCTC
TACTCGCTGTCAAGCGTGGTCACCGTCCCGAGCTCATCCCTGGGTACTCAGACCTACATTTG
CAACGTGAACCACAAACCTTCCAACACCAAGGTCGACAAGAAAGTGGAGCCTAAGAGCTGCG
ACAAGACCCACACCTGTCCCCCGTGTCCCGCCCCTGAGCTGCTGGGCGGCCCCAGCGTGTTC
CTCTTCCCGCCTAAGCCGAAGGACACTCTGATGATCTCGAGAACCCCTGAAGTGACCTGTGT
GGTGGTGGATGTGTCCCACGAGGATCCGGAAGTGAAGTTCAATTGGTACGTGGACGGAGTGG
AAGTCCATAACGCCAAGACCAAGCCCCGCGAGGAACAGTACAACTCAACTTACCGGGTGGTG
TCAGTGCTGACCGTGCTGCACCAAGATTGGCTGAACGGGAAGGAGTACAAGTGCAAAGTCTC
CAACAAGGCGCTGCCGGCCCCCATTGAAAAGACCATCAGCAAGGCTAAGGGCCAGCCCCGGG
AACCACAGGTCTACACCTTGCCCCCTTCCCGGGAGGAAATGACCAAGAACCAAGTGTCGCTG
ACGTGCCTGGTCAAGGGCTTTTATCCATCTGACATCGCCGTGGAGTGGGAAAGCAACGGCCA
GCCGGAAAACAACTACAAGACTACCCCGCCTGTGCTGGACTCCGACGGCTCGTTCTTCCTGT
ATTCCAAGCTCACCGTGGATAAGTCCAGATGGCAGCAGGGCAATGTGTTCAGCTGCAGCGTG
ATGCATGAGGCCCTGCACAACCACTACACTCAGAAATCACTGTCCCTTTCCCCCGGAAAGTA
A
SEQ ID NO: 87: huKappa Constant Nucleotide Sequence
CGAACTGTGGCTGCACCATCTGTCTTCATCTTCCCGCCATCTGATGAGCAGTTGAAATCTGG
AACTGCCTCTGTTGTGTGCCTGCTGAATAACTTCTATCCCAGAGAGGCCAAAGTACAGTGGA
AGGTGGATAACGCCCTCCAATCGGGTAACTCCCAGGAGAGTGTCACAGAGCAGGACAGCAAG
GACAGCACCTACAGCCTCAGCAGCACCCTGACGCTGAGCAAAGCAGACTACGAGAAACACAA
AGTCTACGCCTGCGAAGTCACCCATCAGGGCCTGAGCTCGCCCGTCACAAAGAGCTTCAACA
GGGGAGAGTGTTAA
SEQ ID NO: 101: h2731 Complete Heavy Chain Amino Acid Sequence
EVQLLESGGGLVQPGGSLRLSCAASGFTFSNYGMSWVRQAPGKGLEWVASIRSGSGRTYY
SDNVKGRFTISRDNSKNTLYLQMNSLRAEDTAVYYCVRYDHYSGSSDYWGQGTLVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPGK
SEQ ID NO: 102: h2731 Complete Light Chain Amino Acid Sequence
DVVMTQSPLSLPVTLGEPASISCKSSQSLLDYDGKTYLNWLLQKPGQSPQRLIYRVTNRD
TGVPDRFSGSGSGTDFTLKISRVEAEDVGVYYCWQGTHFPRSFGQGTKVEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSENRGEC
SEQ ID NO: 103: h2731 Complete Heavy Chain Nucleotide Sequence
GAGGTGCAGCTCTTGGAGTCTGGGGGAGGCTTGGTGCAGCCAGGGGGGTCCCTAAGACTCTC
CTGTGCAGCCTCTGGATTCACCTTCTCCAACTATGGCATGTCCTGGGTCCGCCAGGCTCCAG
GGAAGGGACTGGAGTGGGTCGCTTCTATTCGCTCCGGTAGTGGTAGGACATACTACTCAGAT
AACGTGAAGGGCCGGTTCACAATCTCCAGAGACAATTCCAAGAACACGCTGTATCTGCAAAT
GAACAGCCTGAGAGCCGAGGATACGGCCGTTTATTACTGTGTGCGCTACGACCATTACTCTG
GATCCTCTGACTACTGGGGCCAAGGCACCCTTGTCACAGTCTCCTCAGCCTCCACCAAGGGC
CCATCGGTCTTCCCCCTGGCACCCTCTTCCAAGAGCACCTCTGGGGGCACAGCGGCCCTGGG
CTGCCTTGTCAAGGACTACTTCCCCGAACCGGTGACGGTGTCGTGGAACTCAGGCGCCCTGA
CCAGCGGCGTGCACACCTTCCCGGCTGTCCTACAGTCCTCAGGACTCTACTCCCTCAGCAGC
GTGGTGACTGTGCCCTCCAGCAGCTTGGGCACCCAGACCTACATCTGCAACGTGAATCACAA
GCCCAGCAACACCAAGGTGGATAAGAAGGTTGAGCCCAAATCTTGTGACAAAACTCACACAT
GCCCACCGTGCCCAGCACCTGAACTCCTGGGGGGACCGTCAGTCTTCCTGTTCCCCCCAAAA
CCCAAGGACACCCTCATGATCTCCCGGACCCCTGAGGTCACATGCGTGGTGGTGGACGTGAG
CCACGAAGACCCAGAGGTCAAGTTCAACTGGTACGTGGACGGCGTGGAGGTGCATAATGCCA
AGACAAAGCCGAGAGAGGAGCAGTACAACAGCACGTACCGTGTGGTCAGCGTCCTCACCGTC
CTGCACCAGGACTGGCTGAATGGCAAGGAGTACAAGTGCAAGGTCTCCAACAAAGCCCTCCC
AGCCCCCATCGAGAAAACCATCTCCAAAGCCAAAGGGCAGCCCCGAGAACCACAGGTGTACA
CCCTGCCTCCATCCCGGGAGGAGATGACCAAGAACCAGGTCAGCCTGACCTGCCTGGTCAAA
GGCTTCTATCCCAGCGACATCGCCGTCGAGTGGGAGAGCAATGGGCAGCCGGAGAACAACTA
CAAGACCACGCCTCCCGTGCTGGACTCCGACGGCTCCTTCTTCCTCTATTCCAAGCTCACCG
TGGACAAGAGCAGGTGGCAGCAGGGGAACGTCTTCTCATGCTCCGTGATGCATGAGGCTCTG
CACAACCACTACACGCAGAAGAGCCTCTCCCTGTCTCCCGGGAAA
SEQ ID NO: 104: h2731 Complete Light Chain Nucleotide Sequence
GATGTTGTGATGACCCAGTCCCCACTCTCTTTGCCCGTTACCCTTGGAGAACCTGCCTCCAT
CTCTTGCAAGTCAAGTCAGAGCCTCTTAGATTACGATGGAAAGACATATTTGAATTGGTTGC
TGCAGAAGCCAGGCCAGTCTCCACAGCGCCTAATCTATCGGGTGACCAACCGGGACACTGGA
GTCCCTGACAGGTTCAGTGGCAGTGGATCAGGGACAGATTTCACACTGAAAATCAGCAGAGT
GGAGGCTGAGGATGTGGGAGTTTATTATTGCTGGCAAGGCACACATTTTCCGCGCTCTTTCG
GACAGGGGACCAAGGTGGAAATAAAACGAACTGTGGCTGCACCATCTGTCTTCATCTTCCCG
CCATCTGATGAGCAGCTTAAGTCCGGAACTGCTAGCGTTGTGTGCCTGCTGAATAACTTCTA
TCCCAGAGAGGCCAAAGTACAGTGGAAGGTGGATAACGCCCTCCAATCGGGAAACTCCCAGG
AGAGCGTCACAGAGCAGGACAGCAAAGACAGCACCTACAGCCTCAGCAGCACCCTGACGCTG
AGCAAAGCAGACTACGAGAAACACAAAGTCTACGCCTGCGAAGTCACCCATCAGGGCCTGAG
CTCGCCCGTCACAAAGAGCTTCAACAGGGGAGAGTGT

### EXAMPLES

The following examples have been included to illustrate modes disclosed herein. Certain aspects of the following examples are described in terms of techniques and procedures found or contemplated by the present co-inventors to work well in the practice disclosed herein. In light of the present disclosure and the general level of skill in the art, those of skill appreciate that the following examples are intended to be exemplary only and that numerous changes, modifications, and alterations may be employed without departing from the scope of the disclosure.

“Aducanumab” or “Adu” as used in these experiments refers to an antibody with heavy chain of SEQ ID NO: 70 and light chain of SEQ ID NO: 71, and as set forth in United States patent publication number US 2015/0315267 and PCT publication number WO 2014/089500.

“BAN-2401” and “gantenerumab” as used in these experiments refer to an antibody with heavy chain of SEQ ID NO: 82 and light chain of SEQ ID NO:83 as set forth, e.g., in European patent number EP 1960428B1.

In the following methods, antibody binding profiles to aggregated or fibrillar Aβ are characterized by ELISA, surface plasmon resonance (SPR) and immunohistochemistry (IHC). The ability to mediate phagocytic plaque clearance is evaluated ex vivo in APP/PS1 transgenic mouse brain as well as AD brain with primary murine microglia by immunofluorescence, ELISA and MSD quantification, and neutralization of Aβ oligomer neuronal binding is assessed in rat primary hippocampal cultures.

Results presented herein: relative to other N-terminal Aβ antibody therapies (bapineuzumab, aducanumab), mAbs of the description exhibited greater apparent affinity for aggregated and fibrillar Aβ in competition or standard binding ELISAs. The enhanced avidity of mAbs of the disclosure for fibrillar Aβ was confirmed by SPR equilibrium binding kinetics, indicating 5-11-fold higher avidity than aducanumab due to slower off-rate kinetics. IHC dose response assessments on frozen human AD brain sections showed greater apparent affinity and plaque area binding than aducanumab, regardless of the individual AD donor tissue tested. In ex vivo activity assays, mAbs of the disclosure were shown to significantly facilitate Aβ plaque reduction by microglial phagocytosis in APP/PS1 mouse tissue and to block soluble Aβ oligomer binding to rat primary neurons in a concentration-dependent manner. In ex vivo functional assays with human AD brain, mAbs from the description were shown to significantly facilitate clearance of pyroglutamyled Aβ, a post-translationally modified component of senile plaques.

### Example 1. Aβ Antibody Design

Aβ antibody bapineuzumab (hBP) is a humanized antibody developed from parental murine antibody 3D6. Here, a multipronged approach was applied to construct superior antibodies to hBP. Humanness of hBP was analyzed and a determination was made that light chain humanization could be optimized.

A search was made over the protein sequences in the PDB database [Deshpande et al, 2005] to find structures that would provide a rough structural model of hBP. The crystal structure of hBP fab PDB code 4HIX [Miles, et al., 2013] was utilized for both Vh and Vk structure as it had acceptable resolution (2.2 A) and an exact sequence match to hBP Vh and Vk, retaining the same canonical structures for the loops.

IMGT/DomainGapAlignment was performed for the hBP VL as input sequences. Human germ line VK gene sequence IGHV2-30*02 is the closest matched to hBP VL. The frameworks of hBP VL share a high degree of sequence similarity with the corresponding framework regions of IGHV2-30*02. Thus, the framework regions of IGHV2-30*02 VL were chosen as the guidance sequence for further optimization of the hBP framework regions. Additionally, three residues in CDR-L2 that do not make any direct contact with the antigen as per hBP 3D structure were also changed to germline sequence resulting in following changes, L50K, K53N and L54R (Kabat).

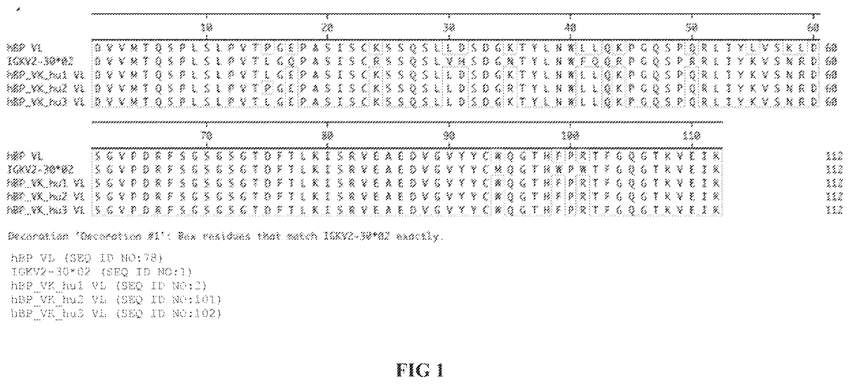

Three different versions of VL were designed by incorporating human germline framework residues into hBP VL sequence. Canonical or interface residues were not changed. An alignment of designed VK version designed is shown in FIG. 1.

Based on structural observation that P15 is located at a turn and the germline gene has Leu at this position, P15L was tested in one version of the variable light chain.

Based on the 3D structural observations, substitutions at a number of residues in the light chain and heavy chain CDRs and framework were designed. In total thirty-one light chain and thirty-two heavy chain mutant VL and VH versions were generated and tested for binding in the first round of rational design. Mutations that showed improved binding were combined in the second round of the rational design. Additionally, new mutations guided by further analysis of the structure were also incorporated into the design.

Rational design based mutagenesis was done for following positions within CDR-H1, T28, S30, N31, Y32 and G33 (Kabat). For CDR-H2 positions 151, G53, G54, T57, S60, D61 and N62 were also mutated (Kabat). CDR-H3 positions D96, H97, S99, S100a and Y102 were subjected to rational mutagenesis (Kabat).

For variable light chain, multiple substitutions were tried at CDR-L1 positions K24, L27c, D27d and S27e (Kabat). Light chain CDR-L2 positions K53 and L54 were subjected to directed and limited mutagenesis (Kabat). CDR-L3 positions were not subjected to substitutions.

A select few positions in the framework regions were also subjected to rational mutagenesis for heavy chain as well as light chain.

Fifty-seven additional heavy chain and thirty-three light chain variants were designed and analyzed with assistance of Atum GPSpro software, which analyzes database of human variable heavy and light chains and, based upon computer learning, suggests query sequence-specific changes.

For the variable heavy domain, a number of substitutions at positions A24, S25, G26, F27, T28, F29, S30, N31, Y32, G33 and M34 were designed and analyzed (Kabat). A majority of these positions were within CDR-H1. Similarly, many of the CDR-H2 residues were subjected to mutagenesis, such as positions A49, S50, 151, R52, S52a, G53, G54, G55, R56, T57, Y58, Y59, S60, D61, N62, V63 and K64 (Kabat). Additionally, multiple substitutions for the amino acids within CDR-H3 were made, for example, positions V93, R94, Y95, D96, H97, Y98, S99, G100, S100a, S100b, D101 and Y102 (Kabat).

Multiple substitutions were also designed for variable light chain CDR-L1 positions K24, S25, S26, Q27, S27a, L27b, L27c, D27d, S27e, D28, G29, K30, T31, Y32, L33 and N34 (Kabat). For CDR-L2, mutagenesis was performed at positions L50, V51, S52, K53, L54, D55 and S56 (Kabat). The majority of CDR-L3 positions such as Q90, G91, T92, H93, F94, P95, R96 and T97 were also rationally substituted with multiple amino acids (Kabat).

All variant antibodies resulting from rational as well as GPSpro design were analyzed for expression, melting point (Tm), affinity, and avidity. Eight antibodies from the rational design and six antibodies from the computer learning campaign were selected for further analysis based on the assays mentioned above.

### Example 2. IC₅₀ Ratio Determination by Competitive ELISA Assays

An assay based on the competition (inhibition) of binding of a labeled antibody to an antigen-coated plate was used to determine IC₅₀ for antibodies of the disclosure.

To generate fibrils, Aβ 1-42 polypeptides, previously treated with HFIP (hexafluoroisopropanol) and dried, were resuspended in DMSO to 5 mM, then further diluted to 100 μM with 10 mM HCl. Samples were incubated at 37° C. for 24 h, and then centrifuged to separate soluble and fibrillar species. The pellet was the resuspended in 1× D-PBS to the original volume and sonicated before use.

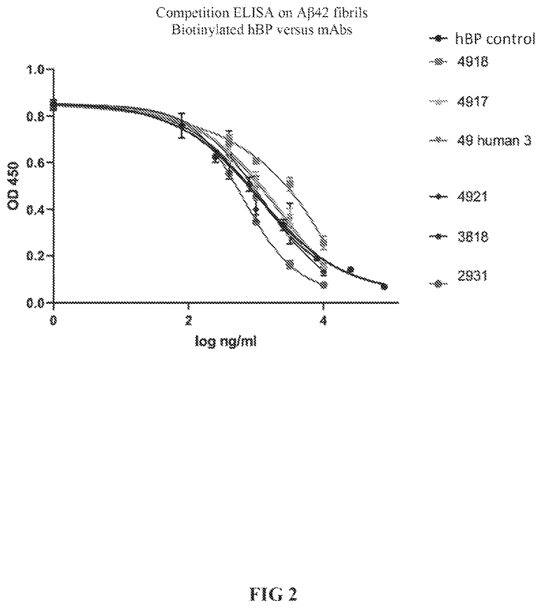

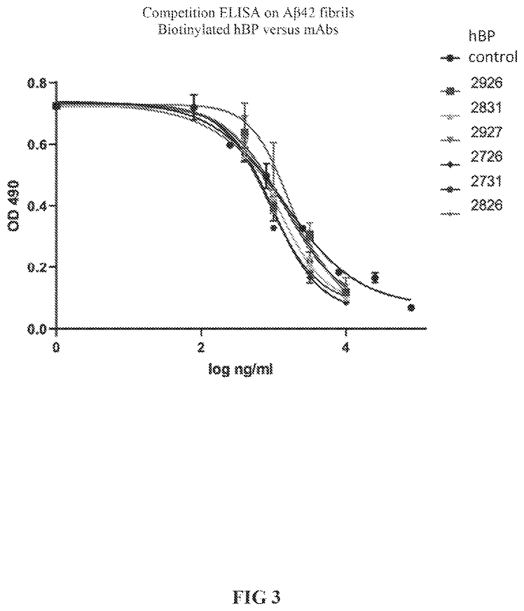

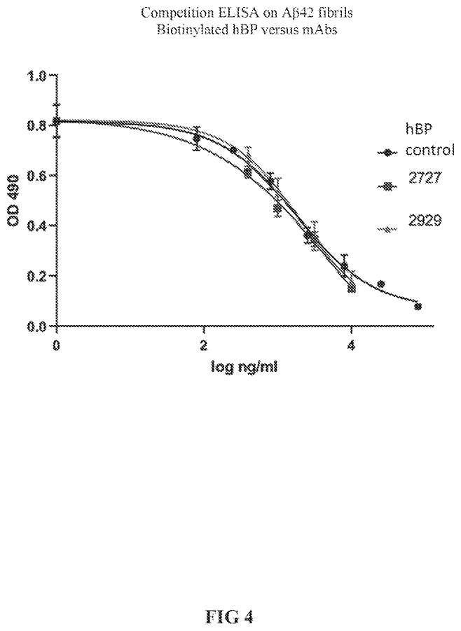

Plates were coated with 0.5 mg/ml of fibril Aβ 42 and blocked, e.g., with 1% BSA/PBS. Seven 3-fold dilutions of hBP starting at 150 μg/ml (75 μg/ml final concentration) and four 3-fold dilutions of test antibody starting at 20 μg/ml (10 μg/ml final concentration) prepared in 0.1% BSA/PBS were added to wells in triplicate, 50 μl per well. 50 ul of hBP-biotin at 0.75 μg/ml (0.35 μg/ml final concentration) prepared in 0.1% BSA/PBS was added to all wells and plates incubated 2 hours at room temperature then washed 3× with TTBS. 100 ul of GE Streptavidin HRP diluted 1/10,000 was then added and incubated for 30 minutes. Plates were then washed 6× with TTBS. Thermo Fisher o-phenylenediamine dihydrochloride (OPD) substrate was prepared fresh per manufacturers direction, and 100 ul per well was added. The reaction was incubated for 15 minutes and the reaction stopped with 50 ul 2N H₂SO₄. Samples were read 490 nM on Spectromax. FIG. 2, FIG. 3 and FIG. 4 illustrate competitive ELISA assay graphs for 4918, 4917, 4921, 3818, 49human3, 2931 and bapineuzumab control (FIG. 2), 2926, 2831, 2927, 2726, 2731, 2826 and bapineuzumab control (FIG. 3) and 2727, 2929 and bapineuzumab control (FIG. 4). IC₅₀ for each test antibody are divided by the IC₅₀ for hBP to yield an half maximal inhibitory concentration (IC₅₀) ratio. A ratio of less than one indicates better performance than hBP. See Table 3A.

TABLE 3A

| | Competition ELISA on |
|---|---|
| | fibril Aβ42 IC₅₀ ratio |
| Antibody | (test:hBP) |
| h2931 | 0.59 |
| h2731 | 0.61 |
| h2726 | 0.68 |
| h2831 | 0.77 |
| h2926 | 0.99 |
| h4921 | 1.01 |
| h2826 | 1.10 |
| h2929 | 1.16 |
| h3818 | 1.18 |
| h2927 | 1.60 |
| h49_hum3 | 2.16 |
| h49_VK17 | 2.69 |
| h2727 | 3.06 |
| h4918 | ND |
| hBP | 1 |

### Example 3. Monoclonal Antibody Potency Determination by Competitive ELISA

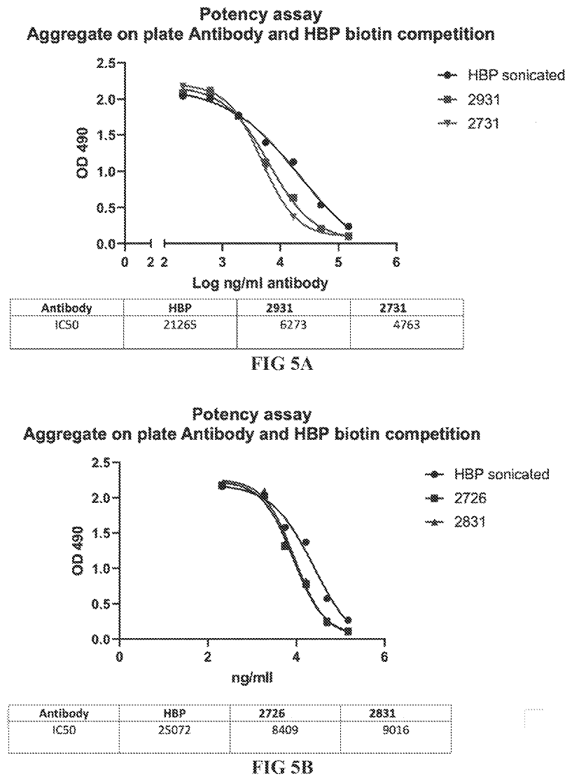

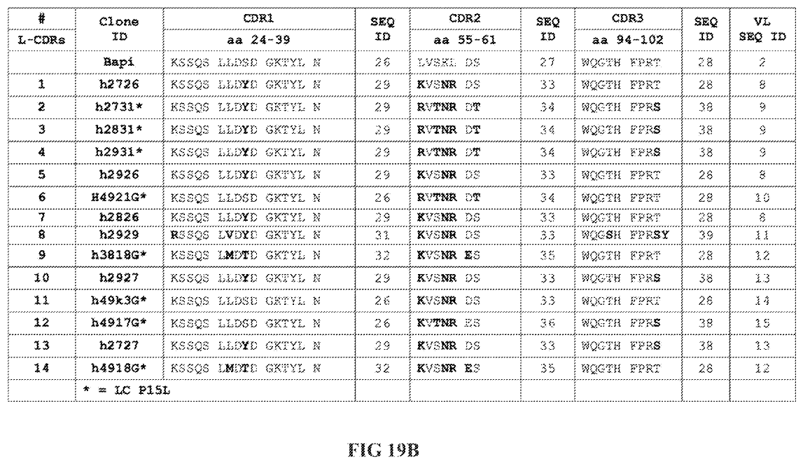

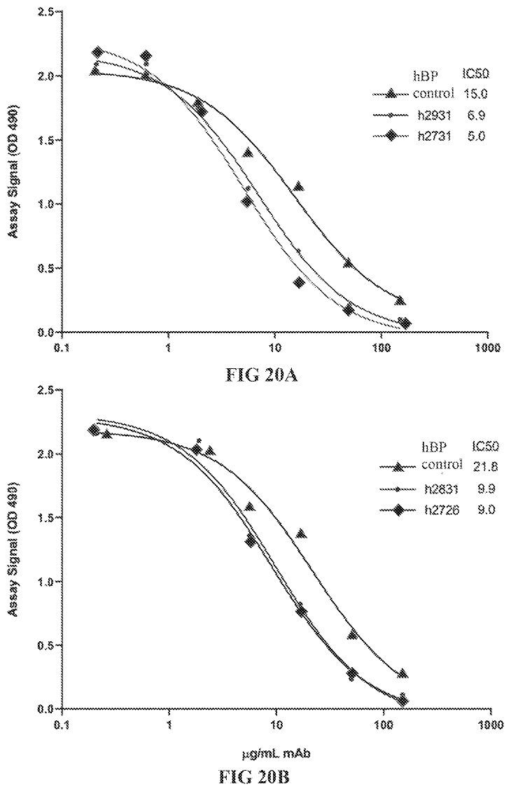

The binding potency of certain monoclonal antibodies of the disclosure and hBP was measured by their ability to compete with biotinylated-bapineuzumab bound to aggregated Aβ42 was assessed by competition ELISA. One mg of Aβ 42 was added to 1 ml of diH2O and was vigorously vortexed and placed on a nutator for 48 hours at room temperature. Plates were coated with 0.5 mg/ml of the heterogeneous Aβ 42 aggregate mixture and blocked, e.g., with 1% BSA/PBS. Seven 3-fold dilutions of hBP starting at 150 μg/ml (75 μg/ml after dilution with hBP-Biotin) and four 3-fold dilutions of test antibody starting at 20 μg/ml (10 μg/ml after dilution with hBP-Biotin) were added to wells in triplicate, 50 ul per well. 50 ul of hBP-biotin at 0.75 μg/ml (0.35 μg/ml after dilution) was added to all wells and plates incubated 2 hours at room temperature then washed 3× with TTBS. 100 ul of GE Streptavidin HRP diluted 1/10,000 was then added and incubated for 30 minutes. Plates were washed six times with TTBS. Thermo Fisher o-phenylenediamine dihydrochloride (OPD) substrate was prepared fresh per manufacturers direction, and 100 μl per well was added. The reaction was incubated for 15 minutes and the reaction stopped with 50 ul 2N H₂SO₄. Samples were read 490 nM on Spectromax. FIG. 5A shows a competitive ELISA assay graph for 2931, 2731 and bapineuzumab control; FIG. 5B shows a competitive ELISA assay graph for 2726, 2831 and bapineuzumab control. FIG. 20A shows a competitive ELISA assay graph for 2931, 2731 and bapineuzumab control (data shown in Table 3B, rows 1-2); FIG. 20B shows a competitive ELISA assay graph for 2831, 2726 and bapineuzumab control (data shown in Table 3B, rows 4-5). For FIG. 20A and FIG. 20B, curves and resulting IC₅₀ estimations represent nonlinear three-parameter least squares fit of data. Individual points are the average of triplicate samples (coefficient of variation <20%).

TABLE 3B

| mAb | Bapi | h2931 | h2731 |
|---|---|---|---|
| IC₅₀ (μg/mL mAb) | 15.04 | 6.901 | 5.024 |
| mAb | Bapi | h2726 | h2831 |
| IC₅₀ (μg/mL mAb) | 21.83 | 9.049 | 9.907 |

Results show that antibodies 2931, 2731, 2726, and 2831 showed greater potency than hBP; ~2-4 lower IC₅₀ values than hBP.

### Example 4. Characterization of Humanized mAbs or Fabs by BIAcore

To compare the binding characteristics of humanized antibodies or humanized antigen-binding fragments (Fab) to recombinant Aβ₁₋₄₂ fibrils, analysis was performed using a BIAcore T200 (GE Life Sciences).

To generate fibrils, Aβ₁₋₄₂ polypeptides, previously treated with HFIP (hexafluoroisopropanol) and dried, were resuspended in DMSO to 5 mM, then further diluted to 100 uM with 10 mM HCl. Samples were incubated at 37° C. for 24 h, and then centrifuged to separate soluble and fibrillar species. The pellet was the resuspended in D-PBS to the original volume and sonicated before use.

Fibrils were immobilized on sensor chip CM5 (GE Healthcare Life Sciences) via amine coupling to a level to ensure a maximum binding of analyte of approximately 100 RU. Various concentrations of antibodies or Fabs (ranging from 1 nM to 100 nM) were passed over the coupled ligand at 30 μL/min in running buffer (HBS+0.05% P-20, 1 mg/mL BSA) for 300 s association time and 1200 s dissociation time. Regeneration of the chip surface was accomplished by 2 short injections of 10 mM Glycine-HCl at pH 1.7. Data was blank-subtracted to both a sensor not containing ligand and 0 nM analyte concentration. Analysis was performed using a global 1:1 fit with BIAcore Insight Evaluation software (v2.0) with bulk refractive index set to zero RU. Off-rate data (k_{diss}; kd) are shown in Table 4 (Fabs) and Table 6 (antibodies).

Similar, small dissociation constants can be seen for the h2726, h2731, h2831 and h2931 Fabs and antibodies in comparison to aducanumab, which demonstrated a significantly larger dissociation constant.

TABLE 4

| Injection | | | | |
|---|---|---|---|---|
| variables | | | | |
| Analyte 1 | 1:1 binding | | Apparent | Rmax |
| Solution | ka (1/Ms) | kd (1/s) | KD (M) | (RU) |
| h2726 | 1.29e+5 | 2.59e−4 | 2.01e−9 | 133.5 |
| h2731 | 1.29e+5 | 2.89e−4 | 2.24e−9 | 134.0 |
| h2831 | 1.08e+5 | 2.48e−4 | 2.31e−9 | 127.1 |
| h2931 | 1.23e+5 | 1.99e−4 | 1.62e−9 | 132.0 |
| hBP | 1.12e+5 | 6.00e−4 | 5.34e−9 | 116.1 |

### Example 5. Characterization of Humanized mAbs Affinity Apparent by BIAcore

Determination of binding affinity of anti-Aβ candidates to Aß₁₋₂₈ (Bachem, Torrance, CA) was performed using a Biacore T200. Anti-human Fc antibody was immobilized to a CM3 sensor chip (GE Healthcare Life Sciences) via amine coupling and used to capture Aß antibodies.

Various concentrations of Aß₁₋₂₈ (analyte, ranging from concentrations of 100 nM down to 0.39 nM, serial diluted 2-fold each dilution step) were passed over the captured ligand at 50 μl/min in running buffer (HBS+0.05% P-20, 1 mg/mL BSA) for 240 s association time and 900 s dissociation time. Data were blank subtracted to both an irrelevant sensor not containing ligand, and buffer runs containing 0 nM analyte concentration. Analysis was performed using a global 1:1 fit with Biacore Evaluation software (v3.0).

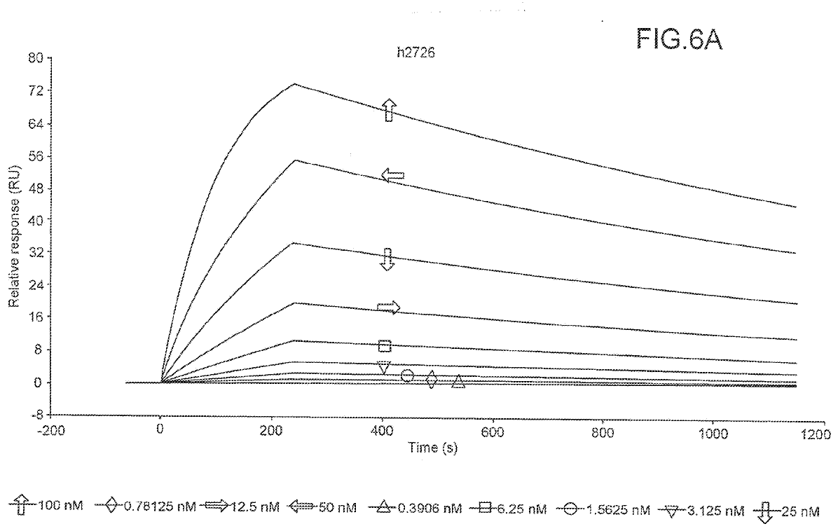

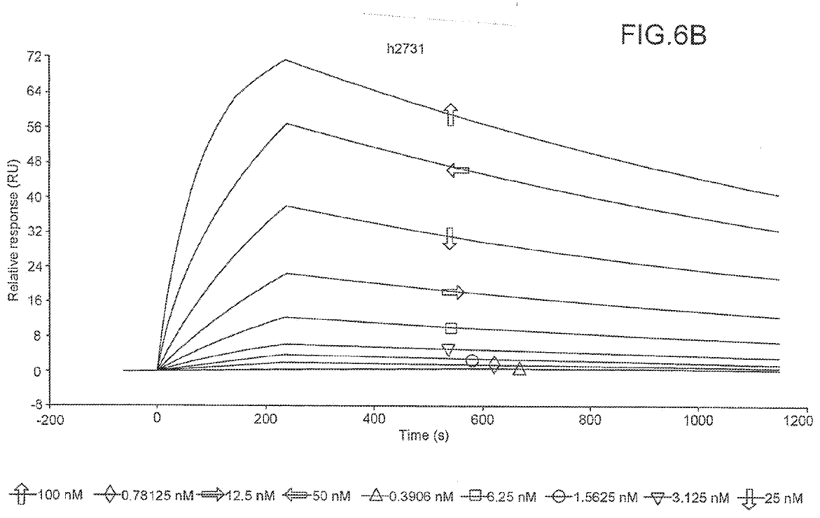

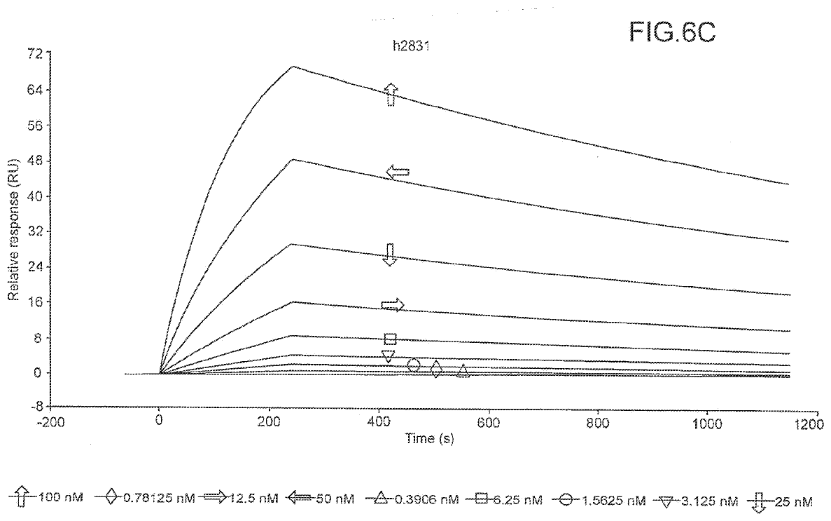

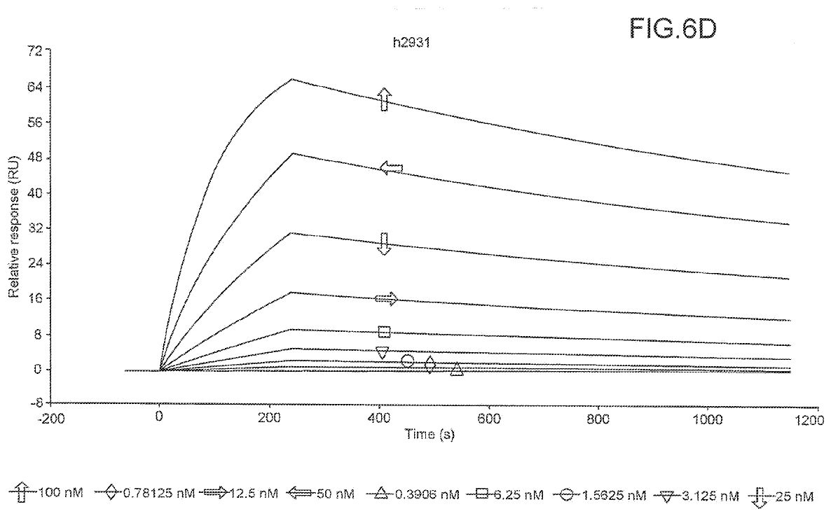

Apparent dissociation constants (KD) are shown in Table 5, where mAbs of the disclosure demonstrated 4-7 nM binding affinity for Aß₁₋₂₈ monomer. Sensorgrams of binding at concentrations from 0.39 nM through 100 nM are shown in FIG. 6A (h2726), FIG. 6B (h2731), FIG. 6C (h2831) and FIG. 6D (h2931).

TABLE 5

| Injection | | | | | |
|---|---|---|---|---|---|
| variables | | | | | |
| Capture | Analyte 1 | 1:1 binding | | Apparent | Rmax |
| Solution | Solution | ka (1/Ms) | kd (1/s) | KD (M) | (RU) |
| h2726 | Aβ₁₋₂₈ | 9.23e+4 | 5.55e−4 | 6.01e−9 | 87.0 |
| h2731 | Aβ₁₋₂₈ | 1.19e+5 | 5.95e−4 | 5.01e−9 | 78.3 |
| h2831 | Aβ₁₋₂₈ | 7.31e+4 | 5.08e−4 | 6.95e−9 | 88.0 |
| h2931 | Aβ₁₋₂₈ | 9.47e+4 | 4.12e−4 | 4.35e−9 | 76.1 |

### Example 6. Characterization of Humanized mAbs Affinity Apparent by BIAcore

To compare the binding characteristics of humanized antibodies to recombinant Aβ₁₋₄₂ fibrils, analysis was performed using a BIAcore T200.

To generate fibrils, Aβ₁₋₄₂ polypeptides, previously treated with HFIP (hexafluoroisopropanol) and dried, were resuspended in DMSO to 5 mM, then further diluted to 100 μM with 10 mM HCl. Samples were incubated at 37° C. for 24 h, and then centrifuged to separate soluble and fibrillar species. The pellet was the resuspended in 1× D-PBS to the original volume and sonicated before use.

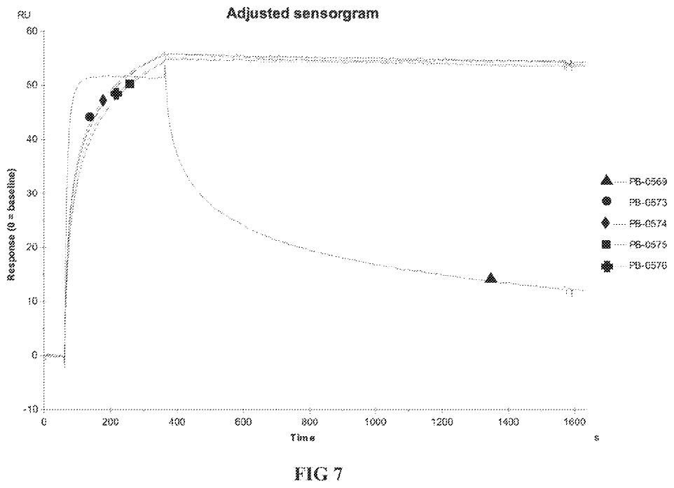

Fibrils were immobilized on sensor chip CM5 (GE Healthcare Life Sciences) via amine coupling to a level to ensure a maximum binding of analyte of approximately 50 RU. Various concentrations of antibodies (ranging from 0.411 nM to 100 nM) were passed over the coupled ligand at 30 μL/min in running buffer (HBS+0.05% P-20, 1 mg/mL BSA) for 300 s association time and 1200 s dissociation time. Regeneration of the chip surface was accomplished by 2 short injections of 10 mM Glycine-HCl pH 1.7. Data was blank subtracted to both a sensor not containing ligand and 0 nM analyte concentration. Analysis was performed using a global 1:1 fit with BIAcore Insight Evaluation software (v2.0) with bulk refractive index set to zero RU. Apparent dissociation constant (KD) are shown in Table 6 and a comparison sensorgram of binding at 100 nM is shown in FIG. 7.

TABLE 6

| | Injection | | | | |
|---|---|---|---|---|---|
| | variables | | | | |
| Immobilized | Analyte 1 | 1:1 binding | | Apparent | Rmax |
| ligand | Solution | ka (1/Ms) | kd (1/s) | KD (M) | (RU) |
| fibril Aβ 7.5 μg/mL | Adu | 2.96e+7 | 1.70e−2 | 5.74e−10 | 45.2 |
| Ace4.5 | | | | | |
| fibril Aβ 7.5 μg/mL | h2726 | 3.93e+5 | 2.12e−5 | 5.40e−11 | 51.0 |
| Ace4.5 | | | | | |
| fibril Aβ 7.5 μg/mL | h2731 | 3.72e+5 | 2.62e−5 | 7.04e−11 | 50.7 |
| Ace4.5 | | | | | |
| fibril Aβ 7.5 μg/mL | h2831 | 2.65e+5 | 2.94e−5 | 1.11e−10 | 50.2 |
| Ace4.5 | | | | | |
| fibril Aβ 7.5 μg/mL | h2931 | 3.35e+5 | 2.05e−5 | 6.12e−11 | 50.0 |
| Ace4.5 | | | | | |

Abeta, amyloid beta, Aβ; ka, association rate constant; kd, dissociation rate constant; KD, apparent equilibrium dissociation constant; mAb, monoclonal antibody; Rₘₐₓ, maximum response; SPR, surface plasmon resonance.

The enhanced relative avidity of monoclonal antibodies of the disclosure for fibrillar Aβ observed by ELISA was confirmed by SPR equilibrium binding kinetics (Table 6), which indicated a 5- to 11-fold greater avidity (apparent KD) than aducanumab.

This is explained by the different kinetic binding profiles observed in the SPR sensorgram (FIG. 7). Although aducanumab binds Aβ fibrils at a faster association rate (ka), the much slower dissociation rate (kd) of the monoclonal antibodies of the disclosure resulted in greater measured avidity (i.e., lower KD*) than aducanumab.

### Example 7. Aβ Fibril Binding by ELISA

The direct binding of certain monoclonal antibodies of the disclosure and aducanumab to Aβ₁₋₄₂ and Aβ_{pE3-42} fibrils was assessed by ELISA. To generate fibrils, Aβ₁₋₄₂ or Aβ_{pE3-42} polypeptides, previously treated with HFIP (hexafluoroisopropanol) and dried, were resuspended in DMSO to 5 mM, then further diluted to 100 μM with 10 mM HCl. Samples were incubated at 37° C. for 24 h, and then centrifuged to separate soluble and fibrillar species. The pellet was the resuspended in 1× D-PBS to the original volume and sonicated before use.

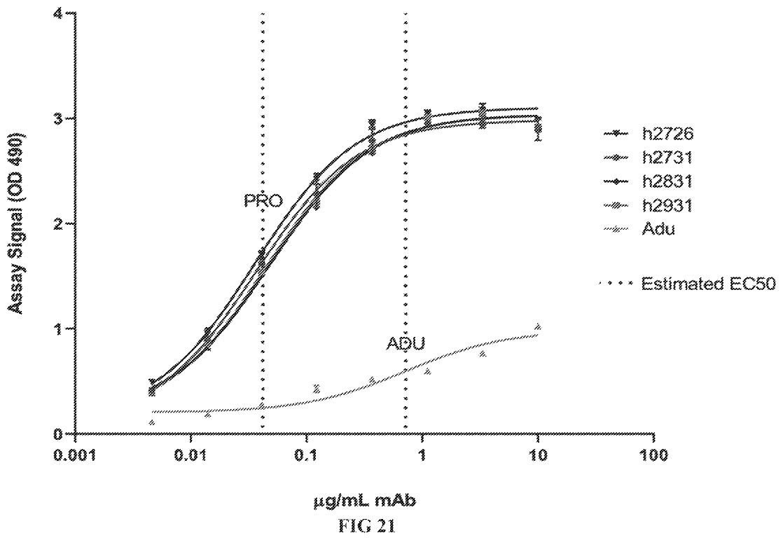

1.0 μg/ml or 2.5 μg/ml of Aβ fibrils in PBS were coated overnight at room temperature. Plates were blocked 1% BSA/PBS for 1 hour. Antibodies were serially diluted from 10 μg/ml to 4.8 ng/ml in 0.1% BSA-PBS and 0.1% Tween 20 and 100 μl of each dilution was added in duplicate to each antibody and incubated for 2 hrs at room temperature. Plates were washed four times with TBS/Tween 20 and 100 μl of goat anti Human IgG HRP (Jackson ImmunnoResearch Laboratories, Inc, West Grove, PA or Invitrogen, Carlsbad, CA) at 1/5000 dilution was added to each well and incubated 1 hour at room temperature. Plates were wash six times in TBS/Tween 20, and Thermo Fisher o-phenylenediamine dihydrochloride (OPD) tablets and ThermoFisher substrate buffer were prepared per manufacturer's instructions. 100 μl of substrate was added and incubated 15 min. Reaction was stopped with 50 μl H₂SO₄. Plates were read at 490 nm on a molecular devices spectromax. FIG. 9A, and FIG. 21. For FIG. 21, curves and resulting EC₅₀ estimations represent nonlinear three-parameter least squares fit of the data (data shown in Table 7).

TABLE 7

| mAb | h2726 | h2731 | h2831 | h2931 | Adu |
|---|---|---|---|---|---|
| EC₅₀ | 0.0359 | 0.03671 | 0.04894 | 0.04495 | 0.7241 |
| (μg/mL mAb) | | | | | |

Plates were coated with dilutions of Aβ fibrils in PBS from 10 μg/ml to 4.8 ng/ml overnight at room temperature. Plates were blocked 1% BSA/PBS 1 hour. Antibodies at 2 μg/ml in 0.1% BSA/PBS 0.1% Tween 20 were added in duplicate to the appropriate wells and incubated for 2 hrs at room temperature. Plates were washed 4× with TBS/Tween 20 and then 100 μl of Jackson Goat anti Human IgG HRP 1/5000 dilution was added to each well and incubated 1 hour at room temperature. Plates were wash six times in TBS/Tween 20, and Thermo Fisher o-phenylenediamine dihydrochloride (OPD) tablets and Thermofisher substrate buffer were prepared per manufacturer's instructions. 100 μl of substrate was and incubated 15 min. Reaction was stopped with 50 μl H₂SO₄. Plates were read at 490 nm on a molecular devices spectromax. FIG. 9B, right panel.

Antibodies h2726, h2731, h2831 and h2931, all demonstrated strong affinities to fibrils, with the difference between best and worst performer within 25%. Additionally, these four antibodies all demonstrated significantly greater avidity than aducanumab. For FIG. 21, a 3-fold increase in assay signal (OD490) and a 15 to 20-fold lower estimated EC₅₀ indicated increased overall binding and relative avidity of h2726, h2731, h2831 and h2931 mAbs to fibrillar Aβ relative to aducanumab.

### Example 8. H2931 Binding of Aβ Oligomer by ELISA

The direct binding of h2931 to Aβ oligomer was assessed by ELISA. To generate oligomers, first lyophilized biotinylated and unlabeled Aβ (Bachem) were each solubilized at 1 mg/mL in 1,1,1,3,3,3-hexafluoroisopropanol (HFIP, Sigma). HFIP was allowed to evaporate from the samples overnight in a fume hood at room temperature. Aliquots were then centrifuged in a speedvac at room temperature to remove all liquid to generate 250 μg aliquots of HFIP films, which were stored at −80° C. until further use.

Oligomers were prepared by solubilizing 250 μg of biotinylated and unlabeled Aβ HFIP pellets in dry DMSO (Sigma) to a final concentration of 5 mM. For unlabeled:biotinylated mixtures, samples were combined in a 9:1 ratio (unlabeled:biotinylated) in an sterile 1.5 mL low-binding microcentrifuge tube (Axygen). DMSO-solubilized samples were then diluted to 100 μM with cold phenol-free neurobasal media (Invitrogen) and incubated for 24 hours at 4° C. After incubation, the oligomers were separated from large insoluble material via centrifugation at 14,000 g for 15 minutes. The top 90% of the supernatant was carefully removed and placed in a new sterile low-binding microcentrifuge tube and stored on ice until use.

2.5 μg/mL of each preparation in PBS was coated 100 ul per well in Costar ELISA high bind plates overnight at room temperature. Plates were aspirated and then 200 μl of 1% BSA in PBS was added in each well and incubated 1 hour at room temperature. h2931 mAb was made at a starting concentration 10 μg/ml in 0.1% BSA/PBS 0.1% tween 20 buffer and serially diluted seven times (1:2 each time) with the same. The samples were incubated for 2 hours at room temperature. Plates were washed 4 times with TBS.0.1% tween 20. Goat anti-human (H+L) HRP (Jackson Immunoresearch, PA) was diluted 1/5000 in 0.1% BSA/PBS 0.1% tween 20, added at 100 μl/well and incubated 1 hour at room temperature. Plates were washed 4 times and o-phenylenediamine dihydrochloride tablets (ThermoFisher) were prepared as per manufacturer instructions. 100 μl was added per well and incubated for 15 minutes at room temperature. Reactions were stopped by the addition of 50 μl of H₂SO₄, and samples were read at 490 nM on a Molecular Devices SpectroMax. Curves and resulting EC₅₀ estimations represent nonlinear 3-parameter least-squares fit of data using GraphPad Prism software.

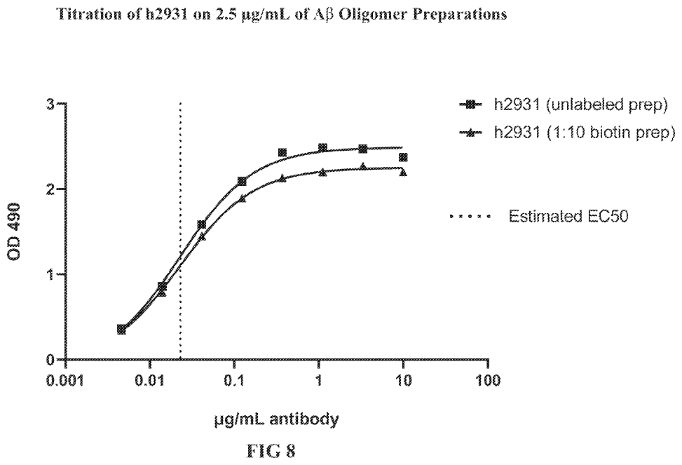

mAb h2931 was shown to bind soluble oligomers with high relative affinity, with an estimated EC₅₀ of 23 ng/mL or 0.15 nM. FIG. 8.

### Example 9. Anti-Aβ Antibodies Binding in AD Brain

Tissue samples. Frozen human AD brain samples were obtained from Banner Sun Health Research Institute, Sun City, AZ. The tissues are from donors who were confirmed to have high amount of Aβ pathology and staged according to the Braak system at the provider institution (Table 8). In addition, quality control was performed in-house on all tissue blocks to ascertain their pathology level and distribution.

TABLE 8
AD donor information

| Case ID | gender | Expired_age | PMI | Braak score |
|---|---|---|---|---|
| AD 13-75 | M | 77 | 3.62 | VI |
| AD 14-11 | M | 82 | 3.98 | V |
| AD 15-19 | F | 83 | 3.62 | V |
| AD 11-97 | F | 86 | 2.52 | V |

Tissue Sectioning and Fixation. The unfixed frozen brain tissue samples were embedded in Tissue-Tek OCT (Sakura Finetek) in cryomolds dipped in a mixture of 2-methylbutane and dry ice slurry (−60° C.) then stored at −80° C. until sectioning. Serial 10 μm thick cryosections were generated using a Leica 3050S cryostat. The sections were directly thaw-mounted on positively charged glass slides and were stored at −20° C. until use. Prior to immunohistochemistry IHC procedures, the slides were immersed in 10% neutral buffered formalin solution for 10 minutes at 4° C., rinsed in PBS, then incubated for an hour at 37° C. in a glucose oxidase solution (20 mM beta D (+) glucose, 2 mM sodium azide, and 2 units/mL glucose oxidase in 1× PBS). The slides were rinsed 3 times for 5 minutes in PBS before they were transferred onto staining racks for processing in an automated stainer.

Antibody biotinylation. The humanized IgG antibodies were biotinylated using a non-covalent method, by means of incubation with a biotin-conjugated goat anti-human monovalent fab fragment (Jackson ImmunoResearch) in a ratio of 1:4, for 1 hour at room temperature. Unbound excess Fab was absorbed by pre-incubation with human serum for an additional hour before use. The freshly prepared antibodies were then loaded into the stainer for immediate application to tissue sections.

Immunostaining. The staining was performed in an automated Leica Bond Rx Stainer (Leica Biosystems), using the Bond Research Kit (DS980, Leica Biosystems) and the avidin-biotin amplified immuno-peroxidase detection system. Each biotinylated anti-Aβ antibody, or a human IgG control, was applied to the sections, at specified concentrations, for one hour and the staining was visualized using the avidin-biotin amplification system (ABC Elite Standard, PK-6100; Vector Laboratories). Hematoxylin counter-staining of nuclei was subsequently applied to sections before dehydration in an ascending series of alcohols, clearing in xylene, cover-slipping, and air-drying.

Tissue imaging. The stained slides were digitally imaged using a Hamamatsu NanoZoomer 2.0HT slide scanner (Hamamatsu Corporation), and the images were captured in an .ndpi file format using the NanoZoomer Digital Pathology software (NDP.scan, Version 2.7.25). Images included in this report were captured directly from NDP.view and transferred without any enhancement. For morphometry, the digitized slides were analyzed using Halo software (V2.1.1537) to measure the percentage of stained tissue, and the results were plotted using GraphPad Prism 8.

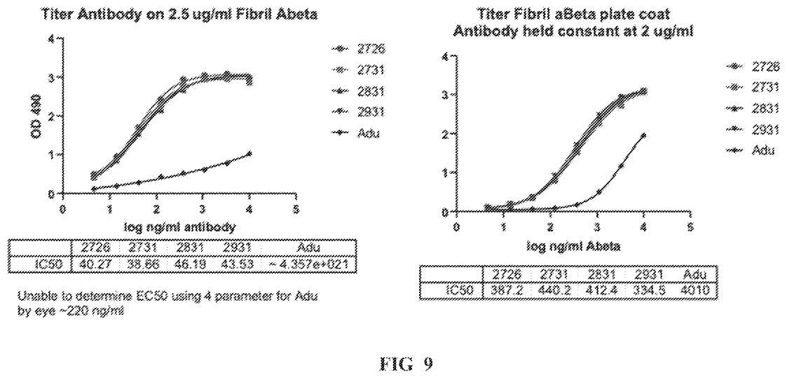

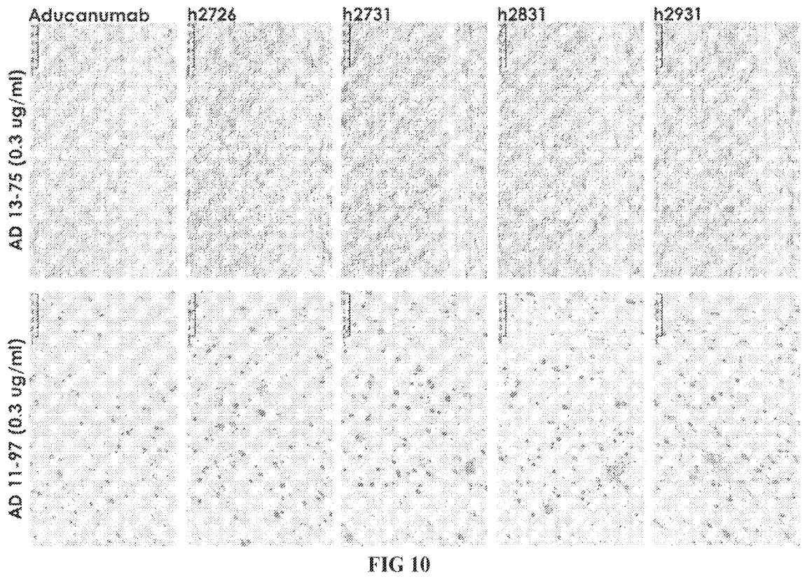

Results with h2726, h2731, h2831, h2931 and aducanumab. Four humanized anti-Aβ antibodies of the disclosure, h2726, h2731, h2831 and h2931, as well as aducanumab, were applied to all four AD brains at increasing concentrations: 0.03, 0.1, 0.3, 1, 3 and 9 μg/ml. As shown in FIG. 10 (0.3 μg/mL), the AD brain sections that were incubated with these antibodies exhibited immunopositive structures that are typical for Aβ pathology in AD. Brains AD 13-75 and AD 14-11 have high density of Aβ plaques while the pathology in brains AD 11-97 and AD 15-19 was comparatively sparse. In each brain, the staining produced by the four antibodies, h2726, h2731, h2831 and h2931, at a specific concentration, was comparable in intensity and distribution. Staining with aducanumab was the weakest among samples and concentrations. As exemplified in FIG. 11, sections from all four brains that were incubated with control human IgG isotype at 1 or 9 μg/ml had no pathology staining.

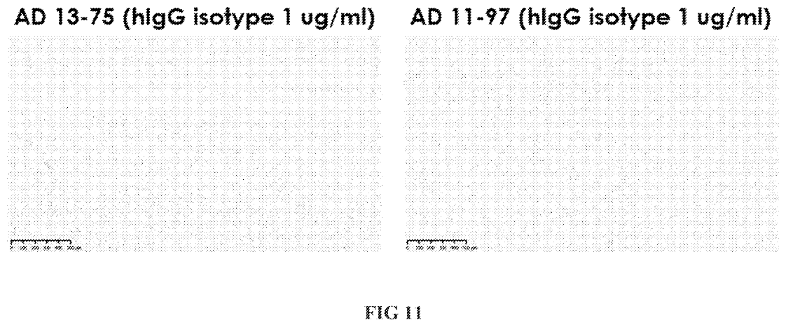

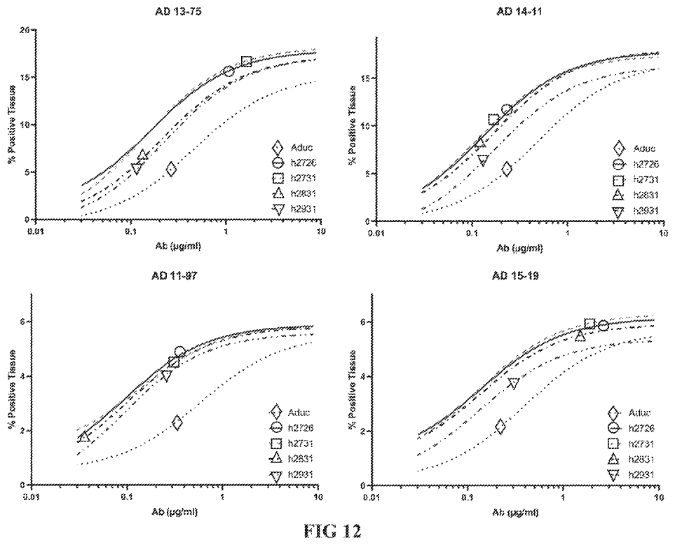

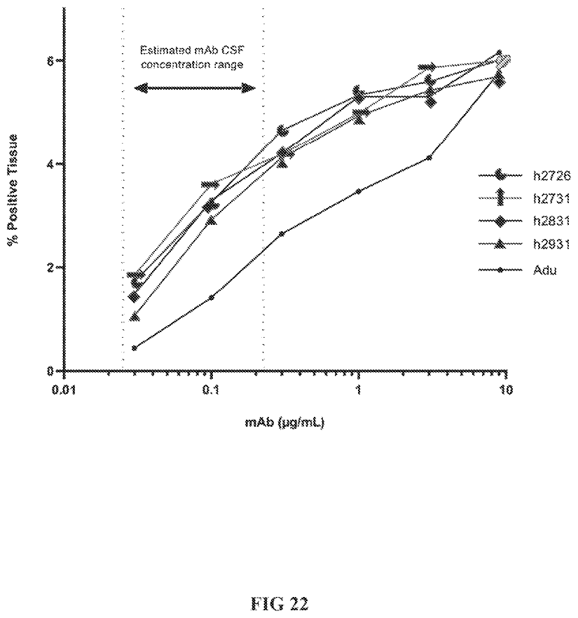

The graphs in FIG. 12 and FIG. 22 are plots of the quantification of staining by the five antibodies in all four AD brains. Measurements of the percentage of tissue surface area that was occupied by the stained pathology confirm that, in each AD brain, the four antibodies, h2726, h2731, h2831, h2931, have similar levels of binding, at all concentrations tested. Correspondingly, the data in Table 9 show that, with each brain, the area under the curve and EC₅₀ values remain comparable for the four antibodies. Values obtained with aducanumab were consistently lower among AD brains throughout the concentration range tested.

FIG. 22 showed greater plaque area binding (as a percentage positive tissue stained) than aducanumab, notably, at antibody concentrations that are estimated to be clinically relevant exposures in cerebrospinal fluid with 10 mg/kg aducanumab. Similar plaque area staining was observed at the highest concentration tested, suggesting saturation of binding at this level.

TABLE 9
Area under the curve and half maximal
effective concentration (EC₅₀)

| | h2726 | h2731 | h2831 | h2931 |
|---|---|---|---|---|
| Area Under | | | | |
| Curve | | | | |
| AD 11-97 | 50.18 | 50.58 | 49.21 | 47.70 |
| AD 15-19 | 52.08 | 52.71 | 49.73 | 44.81 |
| AD 13-75 | 149.3 | 150.4 | 138.7 | 139.1 |
| AD 14-11 | 149.1 | 149.2 | 148.9 | 134.5 |
| EC50 | | | | |
| AD 11-97 | 0.09163 | 0.1346 | 0.1019 | 0.08893 |
| AD 15-19 | 0.1356 | 0.1274 | 0.1328 | 0.1330 |
| AD 13-75 | 0.1615 | 0.1415 | 0.2144 | 0.2273 |
| AD 14-11 | 0.1325 | 0.1102 | 0.1625 | 0.1691 |

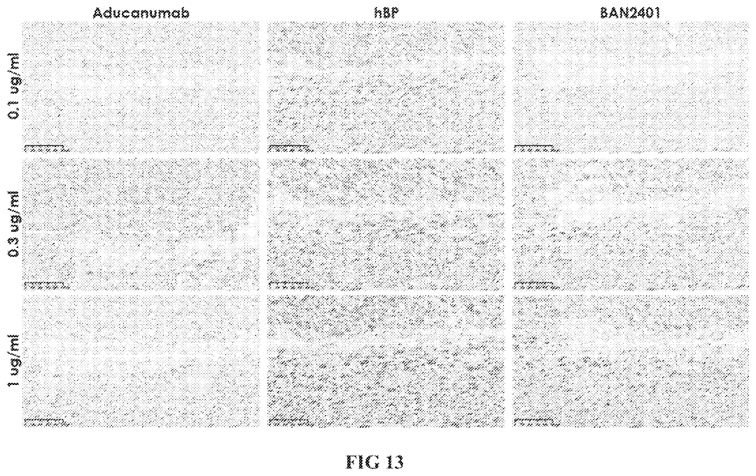

Results with bapineuzumab (hBP). Section from brain AD 13-75 were incubated with the humanized antibody hBP as well as aducanumab and BAN2401 at increasing concentrations: 0.03, 0.1, 0.3, 1, 3 and 9 μg/ml. As seen with antibodies h2726, h2731, h2831 and h2931, the level of staining with hBP increased in a dose dependent manner. In addition, hBP staining was stronger than that of aducanumab and BAN2401 at all concentrations tested, as shown in FIG. 13.

### Example 10. Ex Vivo Phagocytosis Assays for Determination of (Aβ₁₋₄₂ and Aβ_{pE3-42}) Plaque Clearance

In the early stages of AD, microglial function is neuroprotective, acting to clear apoptotic cells and pathological protein aggregates, as well as forming a barrier around plaques to restrict their growth and diffusion of synaptotoxic Aβ oligomers. Ex vivo phagocytosis assays quantitate the antibody-mediated microglial clearance response.

Primary microglial culture generation: For dissection of neonatal mouse brain tissue, P1 pups are quickly decapitated with sterile scissors. Meninges are removed and forebrain were immediately immersed into 1-5 ml dissection media (e.g., high glucose DMEM with 20% FBS, P/S) on ice until the desired number of pup brains has been dissected. Preferably limit total procedure time to within 10 minutes to minimize cellular damage.

Tissue was carefully aspirated twice consecutively with new sterile pipettes using a 22G needle, followed by a 25G needle. Sample were centrifuged at 2,500× g for five minutes at 4° C. Supernatant was carefully aspirated and 5 ml of fresh growth media was added (high-glucose DMEM, 10% FBS, P/S and 25 ng/ml recombinant mouse GM-CSF) to the cell pellets. The cell pellets are pipetted up and down approximately 10 times with a sterile 10 ml pipette to dissociate the pellets.

A cell strainer (100 μm pores) was placed onto a fresh 50 ml conical tube and the material was dispensed through the cell strainer into the conical tube. The cell strainer was rinsed with 4-5 ml of fresh media, followed by centrifuging 200× g for five minutes at 4° C.

Cells were plated at a density of two mouse brains per T-75 plastic culture flask. Carefully aspirate supernatant and add 3 ml of fresh growth medium (high-glucose DMEM, 10% FBS, P/S, and 25 ng/ml recombinant mouse GM-CSF) to each cell pellet with 10 ml sterile pipette. Pipette up and down 10 times with a 10 ml pipette to resuspend. Prepare 1 sterile T-75 flask by adding 6 ml of growth medium (high-glucose DMEM, 10% FBS, P/S and 25 ng/ml recombinant mouse granulocyte-monocyte colony-stimulating factor) into each flask, followed by the addition of 6 ml of resuspended cell pellets to obtain 12 ml final in a 5% CO₂ incubator at 37° C.

Flasks are incubated undisturbed for five days to allow cells to attach. On the fifth day, the culture media was replaced in each flask with 12 ml of fresh growth medium (high-glucose DMEM, 10% FBS, P/S and 25 ng/ml recombinant mouse GM-CSF). Approximately 10% of the mixed cells plated will attach and grow on the plastic surface. The media was changed twice per week (every 3-4 days) to achieve confluence. Such changes are carried out with very carefully without touching the bottom of the flasks where the cells are attached.

After 7-11 d the flasks were rotated at 200 rpm using a Lab-Line orbital shaker with a 19-mm orbit for 2 h at 37° C. Cell suspensions were centrifuged at 200× g and resuspended in assay medium (hybridoma-serum free medium H-SFM [Life Technologies] plus 1% FBS, glutamine, P/S, and 5 ng/ml recombinant mouse GM-CSF).

Ex vivo assays. Cryostat sections (10 μm in thickness; use the wide blades) of APP/PS1 mouse or human AD brains (postmortem interval, less than 3 h) were ‘thaw mounted’ onto polylysine-coated, round glass coverslips and placed in wells of 24-well tissue culture plates (CT −30C OT −20C). Tissue samples can be warmed with thumb in between sections or by reducing OT to −12C). The coverslips were washed twice with assay medium. Antibodies (control or against Aβ) were added at a 2× concentration 250 μl in assay medium (20 μg/ml final) for 1 h in tissue culture incubator.

Microglial cells were then seeded at a final density of 800,000 cells/ml (1,600,000 cells/ml stock) in assay medium 250 μl. The cultures were maintained in a humidified incubator at 37° C. in an atmosphere of 5% CO2 for 72 hrs.

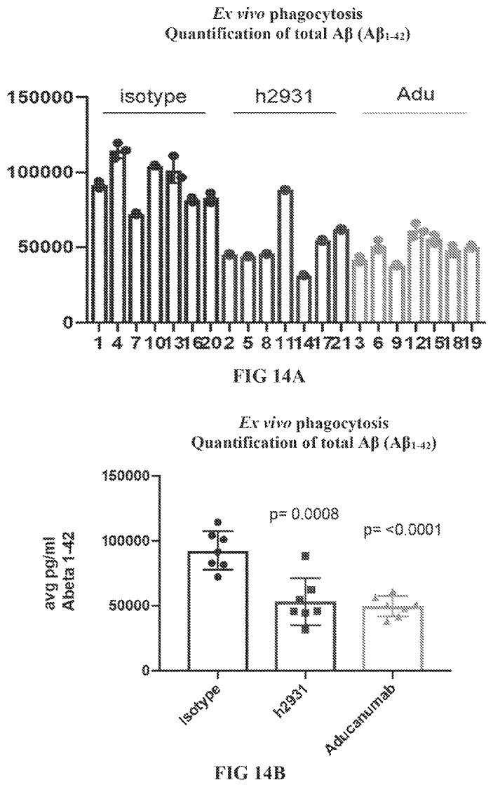

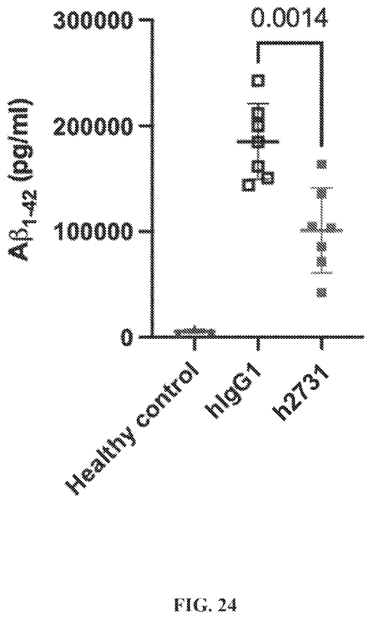

Quantification of total Aβ (Aβ₁₋₄₂). Media was carefully aspirated, followed by washing with ice cold PBS. 100 μl 8M urea was added and tissue resuspended by pipetting and scraped off with pipette tip. Suspension was then frozen at −20° C. until ready for analysis. Suspensions were thawed on ice, centrifuged 16,000× g 20 min at 4° C. before dilution and analysis using a V-PLEX Total Aβ42 Peptide (4G8) Kit (Meso Scale Discovery). Results are shown in FIG. 14A, FIG. 14B, and FIG. 24. FIG. 14A and FIG. 24 show Aβ level per brain section and FIG. 14B shows the same data as a scatter plot per treatment (data for FIG. 14B shown in Table 10; data for FIG. 24 shown in Table 11). h2731, h2931, and aducanumab demonstrated highly significant reductions in Aβ plaque species over isotype control.

TABLE 10

| mAb (Avg. | Isotype | | |
|---|---|---|---|
| pg/mL Aβ₁₋₄₂) | control | h2931 | Aducanumab |
| Mean | 92619 | 53113 | 49501 |
| SD | 14801 | 18239 | 7961 |

TABLE 11

| Condition | Aβ1-42 (pg/ml) | SD |
|---|---|---|
| Healthy control | 5797.25 | 2022.51 |
| AD brain + hIgG1 isotype | 185138.90 | 35888.64 |
| AD brain + h2731 | 101172.05 | 40194.48 |

Quantification of pyroglutamate-3 Aβ (Aβ_{pE3-42}). N-terminal truncated and pyroglutamate-modified Aβ (e.g., Aβ_{pE3-42}) has been described as a component of mature senile plaques in AD brain (Saido et al., Neuron 14, 1995). It was unknown whether pyroglutamate-modification of N-terminal Aβ would affect binding of N-terminal antibodies like h2731 and others described herein. Likewise, it was unknown whether these antibodies would have the ability to promote phagocytic-mediated clearance of Aβ_{pE3-42}.

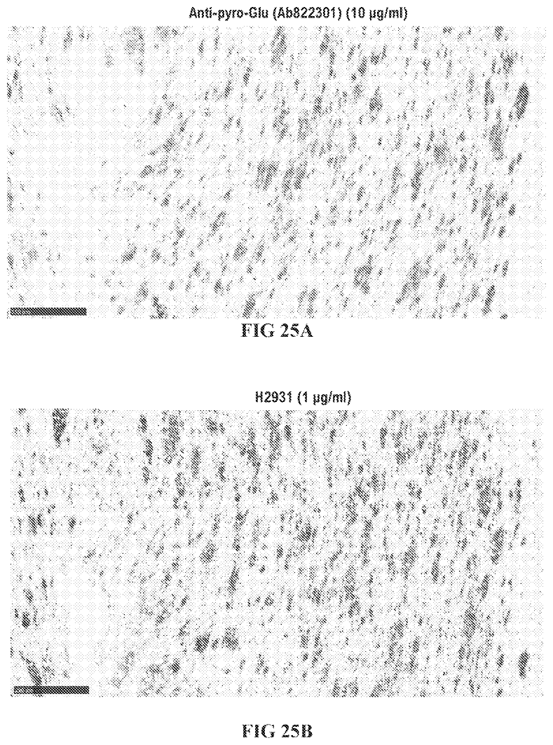

The presence of pyrogulatamate-3 Aβ in AD brain used for ex vivo experiments, as well as its similar staining pattern compared to h2931, was confirmed by immunohistochemistry (FIGS. 25A and 25B). To demonstrate removal of pyroglutamate-3 Aβ, a commercial ELISA method was used to measure its removal during ex vivo phagocytosis. Suspensions that were collected following methods above were thawed on ice, centrifuged 16,000× g 20 min at 4° C. before dilution and analysis using a commercial ELISA kit (Amyloid Beta N3pE Aβ, IBL America). Aβ_{pE3-42} ELISA assay is highly specific to Aβ_{pE3-42} when compared to unmodified Aβ₁₋₄₂ (data not shown).

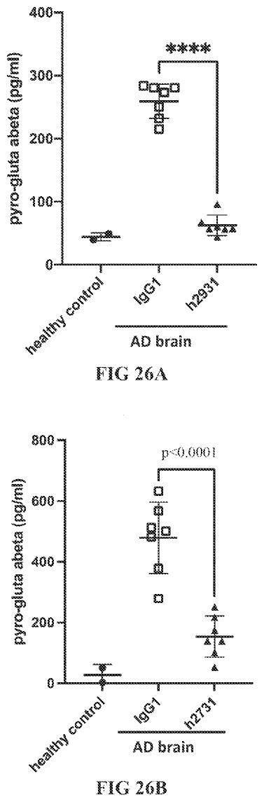

Results are shown in FIG. 26A and FIG. 26B (data shown in Table 12 and Table 13, respectively), which show levels of pyroglutamate-3 Aβ in brain sections after treatment with indicated antibodies, h2931 in FIG. 26A and h2731 in the FIG. 26B, each compared to a healthy control and compared to AD brain treated with IgG1 isotype control. Sections from different AD brains were used for each treatment. h2731 and h2931 both demonstrate highly significant reductions in pyroglutamate-3 Aβ over isotype control.

FIG. 24 and FIG. 26B, taken together, indicate anti-Aβ antibodies of the present invention (e.g., h2731) promote clearance of both Aβ₁₋₄₂ and Aβ_{pE3-42} protein when incubated on AD patient brain tissue sections with primary mouse microglia. These results confirm that these antibodies clear both Aβ₁₋₄₂ and Aβ_{pE3-42} in the human pathology setting.

The N-terminal-targeted anti-Aβ antibodies, facilitated abundant microglia-mediated clearance of Aβ plaque species, including pyroglutamate-modified Aβ, in brain tissue from AD patients. These data support further development of antibodies of the present invention as a subcutaneously administered antibody immunotherapy for Alzheimer's disease.

TABLE 12

| Condition | Ab_{pE3-42} (pg/ml) | Stdev |
|---|---|---|
| Healthy control | 44.20 | 6.39 |
| AD brain + hIgG1 isotype | 259.42 | 27.39 |
| AD brain + h2931 | 62.59 | 16.16 |

TABLE 13

| Condition | Ab_{pE3-42} (pg/ml) | Stdev |
|---|---|---|
| Healthy control | 26.75 | 34.83 |
| AD brain + hIgG1 isotype | 478.91 | 117.80 |
| AD brain + h2731 | 153.76 | 67.59 |

### Example 11. Blocking Oligomers in Hippocampal Binding Assay

Aß Binding Assay in Rat Hippocampal Neurons

E18 primary rat hippocampal neurons were cultured as described by Zago et al. (J. Neurosci 22 Feb. 2012, 32 (8) 2696-2702). Soluble Aß was pre-incubated with and without antibody on culture DIV14-21 to block neuritic binding to primary neurons.

Fresh unlabeled, biotinylated or (9:1) unlabeled:biotinylated soluble Aß was prepared one day prior and incubated overnight at 4° C. The Aß was spun down @ 14,000 RPM for 15 minutes before use.

Each dilution of Aß solution and antibody at (2×) of the final treatment concentration in one-half of final treatment volume using NeuroBasal-no phenol red (NB-NPR) or NbActiv4-NPR medium were prepared. After combining, the mixture was mixed 3-4 times then pre-incubated for 30 minutes at 37° C.

Immediately before binding assay, the neurons were rinsed with pre-warmed NB-NPR at 150 μL/well. The buffer was aspirated and then antibody/Aβ treatment was added to cells at 60 μL/well then incubated for 30-40 minutes at 37° C. under normal incubator conditions (5% CO2; 9% O2).

The neurons were rinsed twice in 150 μL/well NB-NPR then fixed in 4% paraformaldehyde in 1× DPBS for 20 minutes at room temperature.

The cells were permeabilized in 0.1% Triton X-100 in 1× DPBS for 5 minutes and then blocked in 10% normal goat serum (NGS) for 1 hour at room temperature (RT).

The samples were incubated with microtubule-associated protein 2 (MAP2) and neuronal nuclear protein (NeuN) primary antibodies in 100 μL/well 1× DPBS containing 1% BSA+1% NGS overnight at 4° C. On the next day, the samples were rinsed twice in 150 μL/well 1× DPBS for 5 minutes each wash. Secondary antibody was added for 1 hour (@) room temperature in 100 μL/well 1× DPBS+1% BSA+1% NGS.

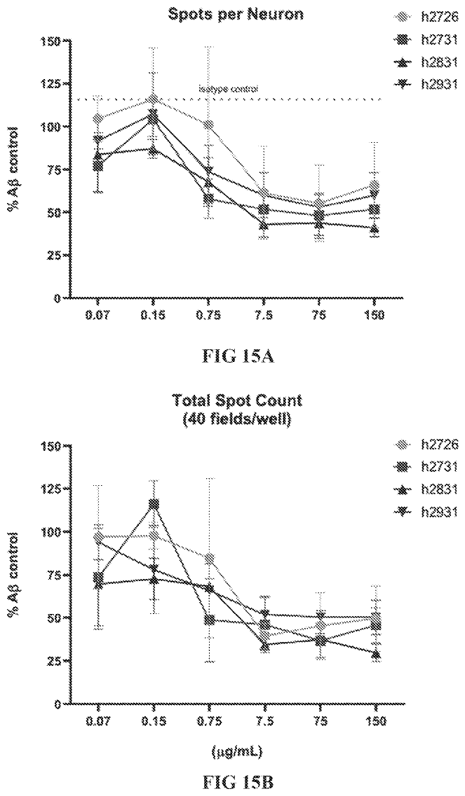

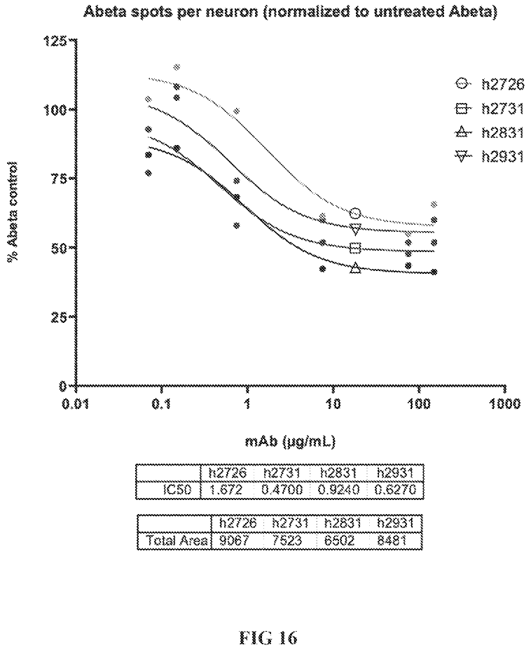

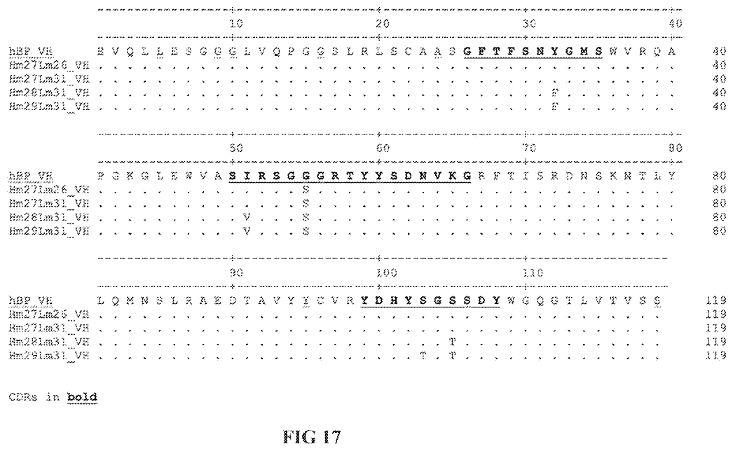

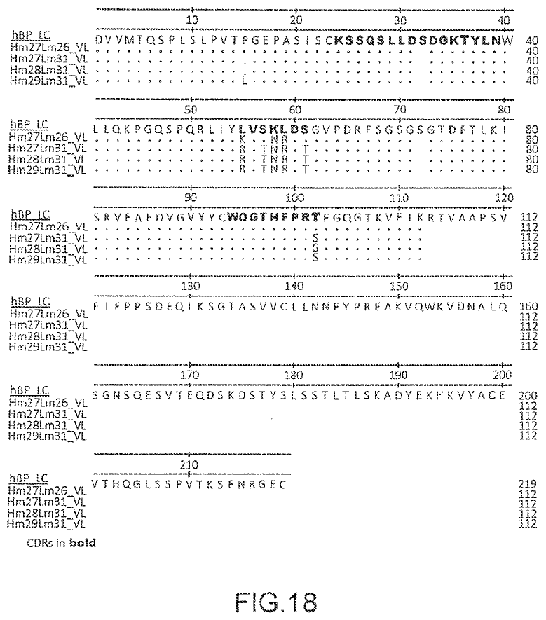

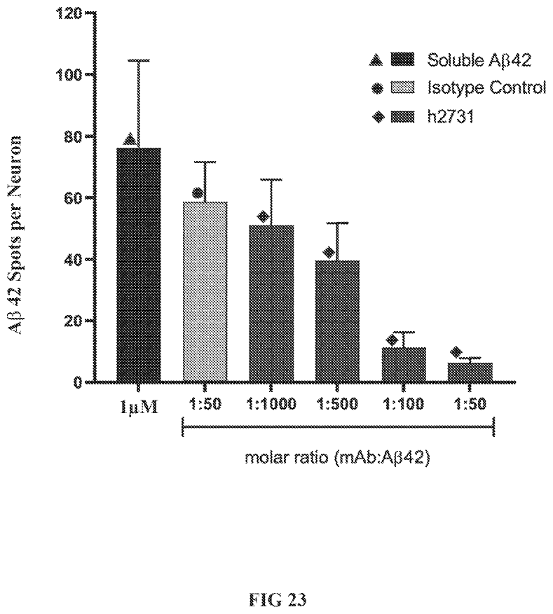

High-content imaging (HCl) analysis was performed to quantify soluble Aβ neuritic binding spots using Operetta HCl CLS instrument (Perkin Elmer; modified Neurite Outgrowth algorithm: 40× H₂O objective; 25-40 fields per well in microplate format; (n=3) per condition. MAP2 and NeuN neuronal markers were used to each trace neurite tree and count cell body number per optical field (e.g., with microtubule-associated protein 2 (Abcam; Cambridge, UK), and NeuN (EMD Millipore) primary antibodies followed by AlexaFluor (Thermo Fisher Scientific) secondary detection antibodies). Neuritic Aß spots were detected using various monoclonal and polyclonal Aß antibodies (e.g., mouse monoclonal anti-Aß antibody MabN254 (EMD Millipore)) followed by AlexaFluor (Thermo Fisher Scientific) secondary detection antibodies or streptavidin-AF488 for biotinylated Aß material. FIG. 15A and FIG. 15B show that increasing concentrations of anti-Aß antibody reduces the number of spots per neuron, indicating activity against Aß. FIG. 23 shows h2731 effectively blocked the binding of soluble Aβ aggregates to rat hippocampal synapses (Aß42 spots per neuron) in a concentration-dependent manner. The effect of h2731 was detected at molar mAb:Aβ42 ratios as low as 1:500 (p<0.05) and reached >90% blockade of binding at 1:50 molar ratios (p<0.001) relative to Aβ42 alone (no mAb preincubation). Data shown in Table 14.

TABLE 14

| | | Isotype | | | | |
|---|---|---|---|---|---|---|
| | Soluble Aβ | Control | h2731 | h2731 | h2731 | h2731 |
| | 1 μM | 1:50 | 1:1000 | 1:500 | 1:100 | 1:50 |
| Mean (Aβ spots | 76.3 | 58.7 | 51.0 | 39.7 | 11.3 | 6.3 |
| per neuron) | | | | | | |
| SD | 28.2 | 12.9 | 14.9 | 12.1 | 4.9 | 1.5 |

### Example 12. Anti-Aß Antibody Binding to Native and Modified Aß Species

Cryostat sections of human AD brain were thaw-mounted onto poly-D-lysine coated coverslips and placed in 24-well tissue culture plates and incubated with test antibodies for 1 hour at 37° C. 5% CO₂. Primary mouse microglial cells were then seeded at 800,000 cells/ml, and the cultures were maintained at 37° C. 5% CO₂ for 72 hours. Media was carefully aspirated, and sections washed with PBS. The sections were resuspended in 8M urea for quantification by ELISA for Aß_{pE3-42} (Immuno-Biological Laboratories, Minneapolis, MN), or MSD for Aß₁₋₄₂ (Meso Scale Diagnostics, Rockland, MD). The Immuno-Biological Laboratories Aß_{pE3-42} ELISA kit specifically detects the pE3-42 species with no detectable signal for full-length Aβ.

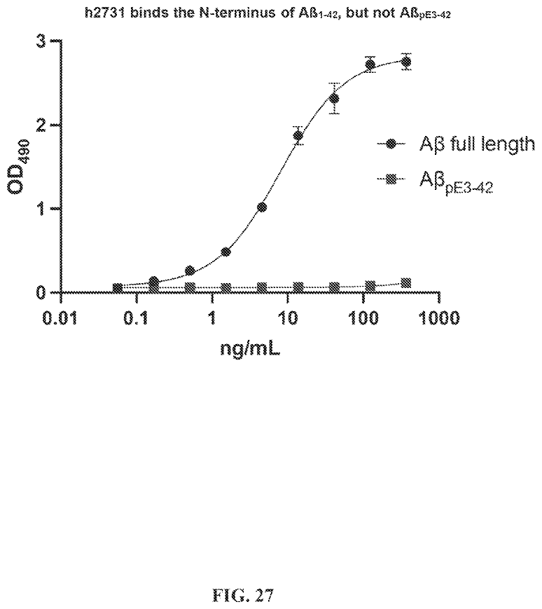

FIG. 27 demonstrates that h2731 binds with high apparent affinity to the N-terminus of full length Aβ but not directly to pyroglutamate-modified Aβ (Aβ_{pE3-42}). h2731 bound with a half-maximal effective concentration (EC₅₀) of 8.1 ng/ml (54 μM) to fibrillar Aβ species with an unmodified N-terminus (Aβ₁₋₄₂). h2731 demonstrated no detectable binding to Aβ_{pE3-42} up to 100 ng/ml.

### Example 13. In Vitro Phagocytic-Mediated Clearance-THP-1 Human Monocyte-Mediated Uptake of Aβ1-42 Protofibrils

Synthetic protofibrils of Aß₁₋₄₂ containing an S26C mutation were generated as described in Paranjape et al., ACS Chem. Neurosci. 2012, 3, 302-311. Briefly, Aβ peptides were dissolved in 100% hexafluoroisopropanol (HFIP) (SigmaAldrich, St. Louis, MO) at 1 mM, aliquoted into sterile microcentrifuge tubes, and evaporated uncovered at room temperature overnight in a fume hood. The following day, the aliquots were vacuum-centrifuged to remove any residual HFIP and stored in desiccant at −20° C. Some Aβ peptides were treated with 100% trifluoroacetic acid and vacuum centrifuged prior to HFIP treatment. Aβ oligomers and fibrils obtained directly from lyophilized aliquots were prepared by resuspending lyophilized Aβ peptide aliquots in sterile anhydrous dimethyl sulfoxide (DMSO) (Sigma-Aldrich, St. Louis, MO) at 5 mM. For oligomer preparation the sample was diluted to 100 μM in sterile ice-cold phenol red-free Ham's F-12 cell culture medium with L-glutamine (F-12, Bioworld, Dublin, OH) and incubated for 24 hours at 4° C. For fibril preparation, the sample was diluted to 100 μM in 10 mM HCl and incubated for 24 hours at 37° C. Aβ concentrations in these preparations were based on dry peptide weight.

Mature protofibrils were conjugated to pHrodo Red Maleimide (Thermo Fisher) before use in in vitro phagocytic-mediated clearance assays.

Antibodies at concentrations of 6.25, 3.13, 1.56, 0.78, 0.39, 0.20, 0.098, and 0.049 μg/ml were preincubated for 30 min at room-temperature with pHrodo-Aß₁₋₄₂ protofibrils, followed by the addition of THP-1 phagocytic cells. After a 3-hour incubation at 37° C. and 5% CO₂, antibody-mediated phagocytic-mediated clearance was assessed by measuring cellular pHrodo signal via flow cytometry.

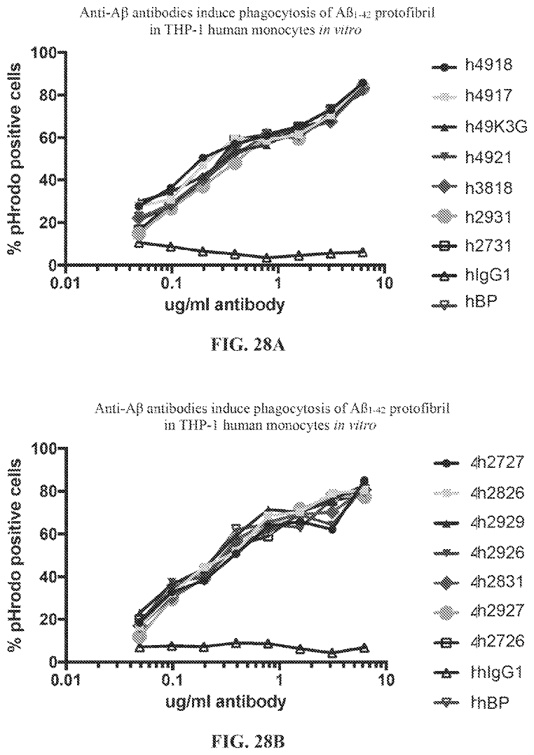

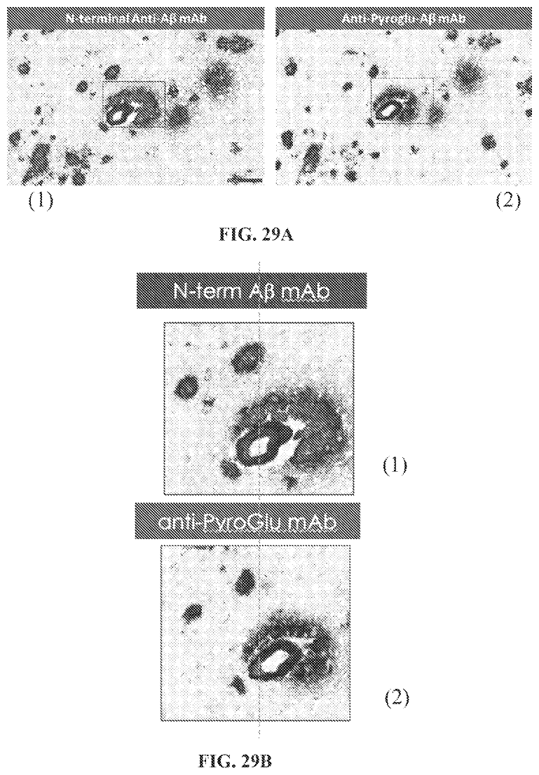

As shown in FIG. 28A and FIG. 28B, anti-Aß antibodies exhibited Aß₁₋₄₂ protofibril phagocytic activity in a concentration-dependent fashion. These results suggest that antibodies of the present invention may be able to drive Aß₁₋₄₂ clearance in brain tissue.

### Example 14. Distribution of Total and Pyroglutamate-Modified Aβ in Brain Tissue from Advanced AD Patients

Ex vivo IHC methods as described above and herein were conducted on AD brain tissue to determine the distribution of Aß_{1-XX} (detected with an N-terminal anti-Aß antibody) and anti-Aß_{pE3-42}.

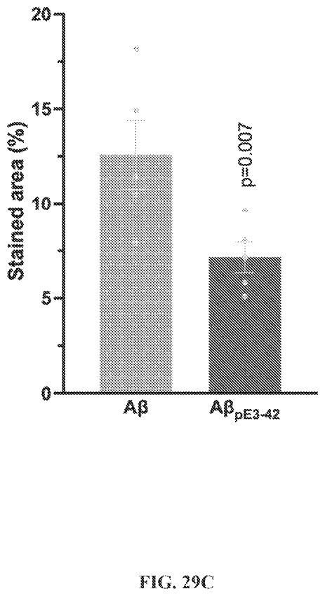

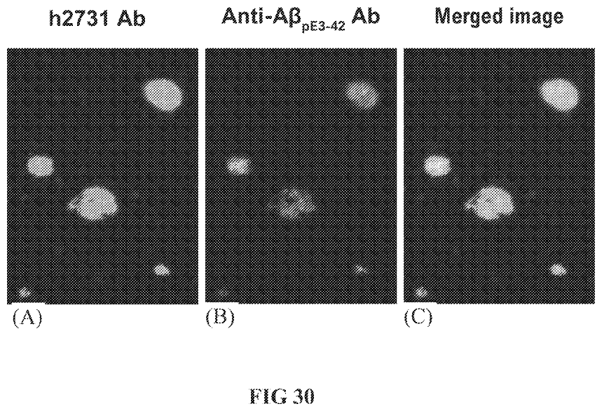

Evaluation of Aß_{1-XX} and Aß_{pE3-42} confirmed widespread distribution of both species in tissue from patients with advanced stage AD. The distribution pattern (FIG. 29A (1) and FIG. 29A (2) (and magnified FIG. 29B (1) and FIG. 29B (2), respectively)) and quantification (FIG. 29C) of the percent area covered by Aß_{1-XX} compared to Aß_{pE3-42} were consistent with prior studies, suggesting that AßpE3-42 represents a relatively smaller pool of modified Aß intermingled with the unmodified Aß targeted by N-terminal Aß antibodies. Aß_{pE3-42} is shown in FIG. 29A(2) and FIG. 29B(2), and intact N-terminal Aß is shown in FIG. 29A(1) and FIG. 29B(1). Anti-Aß_{pE3-42} antibody did not cross-react with Aß₁₋₄₂ (data not shown).

The box in FIG. 29A(1) and FIG. 29B(1) show an Aß plaque with intact N-terminal Aß and modified Aß_{pE3-42} proximal to blood vessel. Table 15 below reports the quantification of staining in plaques in FIG. 29B(1) and FIG. 29B(2) that is presented as graph in FIG. 29C. The difference between the mean values is statistically significant (p=0.007, paired two-tailed t-test).

TABLE 15

| | % Area Stained |
|---|---|
| Antibody | (Mean ± SD; N = 5) |
| anti-N-terminal Aβ antibody | 12.59 ± 4 |
| anti- Aβ_{pE3-42} Aβ antibody | 7.17 ± 1.8 |

### Example 15. Anti-Aβ Antibody h2731 Colocalizes with AßpE3-42 in AD Brain

Colocalization of h2731 immunostaining and Aß_{pE3-42} was assessed by immunofluorescent microscopy. An N-terminal anti-Aß antibody (in this case h2731) was pre-conjugated to a Cy3-secondary anti-human antibody (Jackson Laboratories) before application to tissues. Aß_{pE3-42} was detected using a mouse anti-Aß_{pE3-42} antibody with a 488-AlexaFluor-conjugated anti-mouse secondary antibody. Slides were imaged using a Metamorph-assisted IX81 Olympus microscope connected to a Hamamatsu camera (C10600-10B).

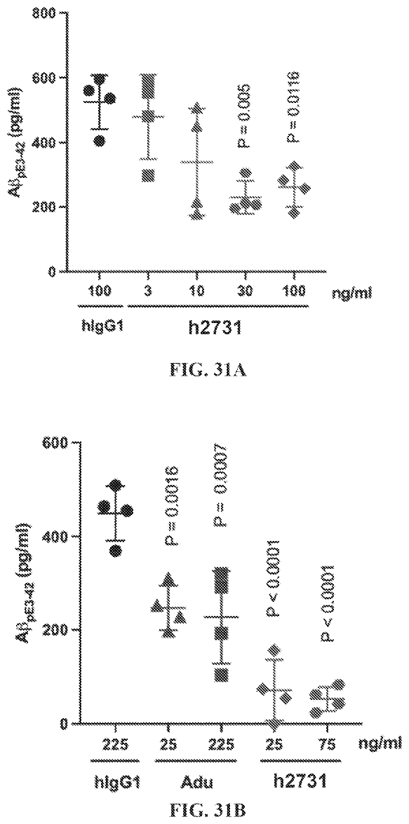

FIG. 30 (panel A) shows localization of h2731 to Aß plaques; FIG. 30 (panel B) shows localization of anti-Aß_{pE3-42} antibody signal to Aß plaques; and FIG. 30 (panel C) shows colocalization of h2731 and anti-Aß_{pE3-42} antibody signal to Aß plaques. Overlapping signal appears more prominent in dense core regions of the plaques.

### Example 16. Anti-Aß Antibodies of the Present Invention Promote Aβ_{pE3-42} Clearance From AD Brain Tissue Ex Vivo in a Dose-Dependent Manner with Higher Efficacy than Aducanumab

Using methods described above and elsewhere herein, the ability of aducanumab and antibodies of the present invention (e.g., h2731) to clear Aß_{pE3-42} protein from AD brain tissue was assessed.

A physiologically relevant dose-response series of h2731 (3 ng/ml, 10 ng/ml, 30 ng/ml and 100 ng/ml) was incubated with AD patient brain tissue sections and primary mouse microglia for 72 hours. h2731 promoted Aß_{pE3-42} clearance in a concentration-dependent fashion. Results are presented in Table 16 below and FIG. 31A.

TABLE 16

| | Concentration | Ave Aβ_{pE3-42} | |
|---|---|---|---|
| Antibody | (ng/ml) | (pg/ml) (n = 4) | Stdev |
| hIgG1 isotype | 100 | 524.34 | 83.36 |
| h2731 | 3 | 479.56 | 129.92 |
| h2731 | 10 | 339.06 | 165.44 |
| h2731 | 30 | 229.28 | 51.16 |
| h2731 | 100 | 261.15 | 60.81 |

h2731 robustly promotes clearance of Aß_{pE3-42} from AD patient brain tissue sections by microglial phagocytosis in a concentration-dependent manner and during a relatively short incubation period (72 hours). Thus, the antibodies of the present invention promote ex vivo clearance of Aβ_{pE3-42} from an AD patient brain at a concentration range expected to be reached with subcutaneous administration.

Another series of experiments were conducted comparing h2731 at 25 ng/ml and 75 ng/ml to aducanumab at 25 ng/ml and 225 ng/ml. Results are presented in Table 17 and FIG. 31B.

TABLE 17

| | Concentration | Ave Aβ_{pE3-42} | |
|---|---|---|---|
| Antibody | (ng/ml) | (pg/ml) (n = 4) | Stdev |
| hIgG1 isotype | 225 | 449.11 | 58.14 |
| Adu | 225 | 227.30 | 98.95 |
| Adu | 25 | 247.34 | 48.06 |
| h2731 | 75 | 52.83 | 25.40 |
| h2731 | 25 | 71.31 | 64.93 |

h2731 exhibited superior Aβ_{pE3-42} clearance activity when compared to aducanumab, even at 9-fold lower concentrations.

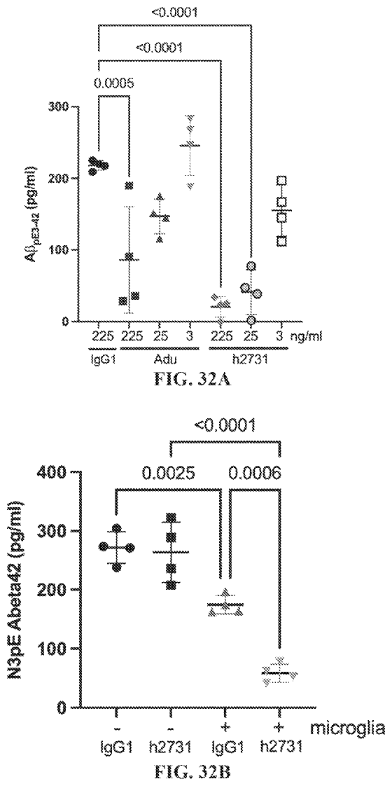

Another physiologically relevant dose-response series of h2731 and aducanumab (3 ng/ml, 25 ng/ml, and 225 ng/ml) was incubated with AD patient brain tissue sections and primary mouse microglia for 72 hours, both compared to IgG1 isotype control. While both h2731 and aducanumab promoted Aß_{pE3-42} clearance in a concentration-dependent fashion, h2731 again did so significantly more potently, with a p-value of <0.0001 at a 9-fold lower concentration than required for aducanumab to reach a p-value of 0.0005. Results are presented in Table 18 below as well as FIG. 32A.

TABLE 18

| | Concentration | AbpE3-42 | |
|---|---|---|---|
| Antibody | (ng/ml) | (pg/ml) | Stdev |
| hIgG1 isotype | 225 | 1.00 | 6.73 |
| Adu | 225 | 85.97 | 74.35 |
| Adu | 25 | 146.70 | 24.30 |
| Adu | 3 | 245.97 | 41.70 |
| h2731 | 225 | 20.60 | 14.44 |
| h2731 | 25 | 41.07 | 31.15 |
| h2731 | 3 | 154.95 | 35.89 |

In order to verify that h2731-mediated ex vivo phagocytosis activity is microglia dependent, a +/−microglia experiment was performed. While microglia alone drive some Aß_{pE3-42} clearance from AD patient tissue sections, clearance is significantly more robust with the combination of h2731 and microglia. h2731 appears to require the presence of microglia for clearance activity, as h2731 alone shows no activity without microglia. Results are presented in Table 19 and FIG. 32B.

TABLE 19

| | Concentration | AbpE3-42 | |
|---|---|---|---|
| Antibody | (ng/ml) | (pg/ml) | Stdev |
| hIgG1 isotype | 75 | 271.79 | 27.01 |
| h2731 | 75 | 263.70 | 51.28 |
| hIgG1 + Microglia | 75 | 174.58 | 15.75 |
| h2731 + Microglia | 75 | 58.37 | 15.53 |

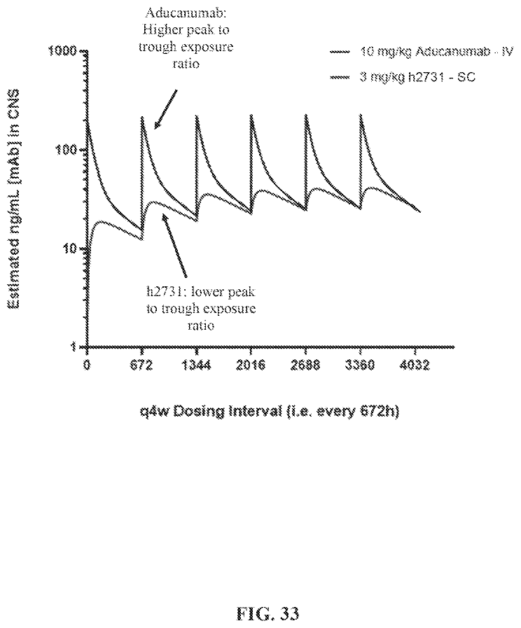

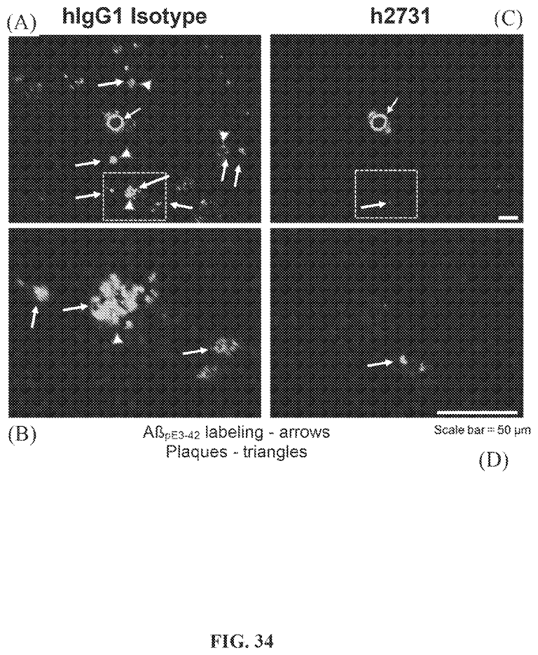

The tested antibody concentrations were based on CNS ranges estimated at 0.1% of steady-state plasma minimum and maximum concentrations from modeled pharmacokinetics following monthly administration of 3 mg/kg subcutaneous h2731 (25-75 ng/ml) or 10 mg/kg of intravenous aducanumab (25-225 ng/ml) in humans (FIG. 33).

Antibodies of the present invention promote ex vivo clearance of Aβ_{pE3-42} from an AD patient brain at a concentration range expected to be reached with subcutaneous administration and with greater biological activity than aducanumab.

Antibody h2731 reduces Aβ_{pE3-42} staining in AD brain. FIG. 34 shows that Aβ_{pE3-42} (staining indicated by white arrows) was observed in plaques (white triangles) and associated with blood vessels (circular shape in FIG. 34A and FIG. 34C) in AD brain treated with human IgG isotype control antibody (FIG. 34A and FIG. 34B). Treatment with h2731 enhanced microglia-mediated reduction of Aβ_{pE3-42} levels as evidenced by the reduction in plaques (FIG. 34C and FIG. 34D). Antibodies of the present invention, as exemplified by h2731, reduce plaques containing Aβ_{pE3-42} in tissue.

### Example 17. H2731 Target Engagement

Female APPxPS1 mice expressing a mutant human amyloid precursor protein (hAPP[V717]) and a mutant human presenilin 1 (hPS1 [A246E]) were used to evaluate the ability of h2731 and aducanumab to traverse the blood-brain-barrier subsequent to peripheral administration and bind to amyloid-beta (Aβ) plaques in the brain. The average age of the animals at the start of the study was 6.7 months. One day prior to drug administration all animals received an injection of an anti-CD4 antibody (20 mg/kg, intravenous) to prevent the formation of anti-drug antibodies in mice receiving h2731 or aducanumab, both of which are fully humanized antibodies. h2731 (3 or 10 mg/kg, subcutaneous, SC) or aducanumab (10 mg/kg, intravenous) were dosed weekly for three weeks and animals were euthanized one week later. Following transcardial perfusion with ice-cold saline, brains were extracted from the mice and flash frozen in 2-methylbutane on dry ice and stored at −80° C.

Serial sagittal 10 μm thick cryosections were generated using a Leica 3050S cryostat. The sections were directly thaw-mounted on positively charged glass slides and were stored at −20° C. until use. Prior to IHC, the slides were immersed in 10% neutral buffered formalin solution for 10 minutes at 4° C., rinsed in PBS, then incubated for an hour at 37° C. in a glucose oxidase solution (20 mM beta D(+) glucose, 2 mM sodium azide, and 2 units/mL glucose oxidase in 1× PBS). The slides were rinsed 3 times for 5 minutes in PBS before they were transferred onto staining racks for processing in an automated stainer. A biotin-SP-conjugated goat anti human IgG (H+L) (Jackson ImmunoResearch Laboratories #109-065-088) was used to detect h2731 or aducanumab in APPxPSI brain tissue. The staining was performed in an automated Leica Bond Rx Stainer (Leica Biosystems), using the Bond Research Kit (DS980, Leica Biosystems). Hematoxylin counter-staining of nuclei was subsequently applied to sections before dehydration in an ascending series of alcohols, clearing in xylene, cover-slipping, and air-drying. The whole sections were imaged using a NanoZoomer 2.0HT slide scanner (Hamamatsu Corporation, Japan). Morphometric analysis of the digitalized images was carried out using Halo software (V2.1.1537). After delineation of the cerebral cortex as region of interest, the percent of stained tissue area was determined. Data are presented in Table 20.

TABLE 20

| | h2731 | | Aducanumab |
|---|---|---|---|
| | 3 mg/kg, SC | 10 mg/kg, SC | 10 mg/kg, IV |
| Plaque Binding | 0.070 ± 0.025 | 0.079 ± 0.034 | 0.060 ± 0.034 |
| (% ROI) | | | |

ROI = region of analysis.
All data represent mean ± SD of n = 5 animals per group

Reduction in numbers or size of Aβ plaques in Alzheimer's disease may correlate with slowing or reversing of disease progression. The ability of the anti-Aβ antibodies of the present invention to bind to and clear Aβ in vivo following peripheral administration supports the potential utility of these antibodies as therapeutic agents.

Thus, the antibodies of the present invention promote microglia-mediated clearance of Aβ₁₋₄₂ in brain tissue from patients with AD. Although antibodies of the present invention may not target the pyroglutamate modification directly, they may effectively clear Aβ_{pE3-42} at concentrations predicted to be clinically relevant and with higher potency and greater biologic activity than aducanumab, as exemplified by h2731. Clearance of pyroglutamate species by these antibodies may be due to the ability of microglia to recognize opsonized plaques and engulf large particles with diverse content. The antibodies of the present invention may therefore clear other neurotoxic elements co-deposited in plaques by this same mechanism.

### Example 18. Reducing Amyloid Plaques in Patients

To reduce amyloid plaques, which has been associated with inhibiting, reducing, and/or reversing the symptoms of Alzheimer's disease, a pharmaceutically effective amount of an anti-Aβ antibody (or antigen-binding fragment thereof) is administered to the patients, such as one or more of the antibodies described in the above examples.

Patients: Individuals that are suspected of having or have been diagnosed with an amyloid plaque-associated disease, such as Alzheimer's disease, are selected for treatment with an anti-Aβ antibody.

Treatment: Patients are administered 70 mg of anti-Aβ antibody h2931 subcutaneously once about every 4 weeks. Patients are administered 200 mg of anti-Aβ antibody h2931 subcutaneously once about every 4 weeks.

Plaque reduction and improvement in symptoms of Alzheimer's disease can be measured as described herein.

A patient with Alzheimer's disease administered 70 mg or 200 mg of anti-Aβ antibody h2731, h2726, h2831, or h2931 subcutaneously once about every 4 weeks is treated by reduction in amyloid plaque burden, measured by PET imaging.

### Example 19. Phase 1 Single Ascending Dose Study to Evaluate the Safety, Tolerability, Immunogenicity, and Pharmacokinetics of h2731

This example describes a phase 1, randomized, double-blind, placebo-controlled, single ascending dose (SAD) study to evaluate the safety, tolerability, and immunogenicity, of h2731 at doses of 70 mg and 200 mg. The purposes of this study is to characterize the plasma pharmacokinetics (PK) profile of h2731 in healthy volunteers (HV) and patients who have AD, and specifically AD patients who have parenchymal amyloid load confirmed by molecular imaging. AD subjects will be required to meet the National Institute on Aging and Alzheimer's Association (NIA-AA) research criteria and guidelines for AD (McKhann, 2011) or mild cognitive impairment (MCI) due to AD (Albert, 2011).

### Study Rational

Preclinical studies in transgenic mice that generate a surplus of Aβ demonstrated that antibodies targeting Aβ N-terminus are able to enter the brain and decrease amyloid deposits in brain tissue and cerebral vasculature (Bard, 2000). Clinical evidence shows that monoclonal antibodies directed towards N-terminus amyloid are able to remove and reduce deposition of Aβ aggregates from the brain, and attenuate cognitive decline (Sevigny, 2016; Swanson, 2021; Aduhelm USPI, 2021).

As shown the examples above, preclinical studies show that h2731 removed Aβ plaques rapidly and robustly by enhancing microglial-mediated clearance mechanisms. These data suggest that h2731 has the potential to slow clinical decline in patients with AD. Doses of 70 mg and 200 mg for Phase 1 clinical testing is based on CNS fractional occupancy modeled from predictions of clinical exposure at these doses.

### Study Objectives

The primary objective of the study is to evaluate the safety and tolerability of h2731 when administered as a single dose, including (1) general safety, tolerability, and immunogenicity in all subjects and (2) target-related safety and tolerability in subjects with confirmed presence of parenchymal amyloid.

A secondary objective of the study is to characterize the PK profile of h2731 and cerebrospinal fluid (CSF) PK profile of h2731 after SC administration as a single dose.

### Study Design

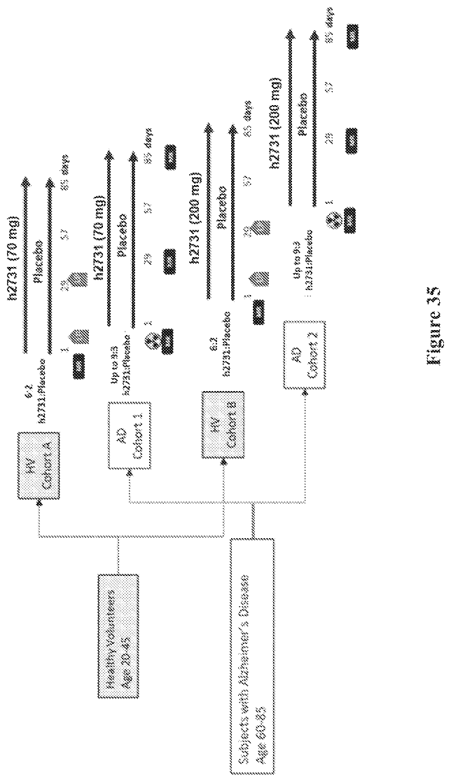

FIG. 35 is a schematic representation of the study plan. The study will comprise at least two dose cohorts of subjects with biologically confirmed AD. The study will further comprise two healthy volunteer (“HV”) cohorts. These four cohorts include the following: AD Cohort 1 (70 mg), AD Cohort 2 (200 mg), HV Cohort 1 (70 mg), and AD Cohort 2 (200 mg), each of which will be dosed subcutaneously (“SC”) with their respective dose.

Following initial on-site observation, subjects will return for four follow-up visits over approximately 12 weeks during which safety assessments and PK collections will be completed. Selected cohorts may additionally undergo CSF collection by lumbar puncture at Days 3 and 29.

### Endpoints

Primary endpoints will include:
Safety and tolerability based on adverse event (“AE”) reporting (incidence of AEs, SAEs, and h2731-related AEs), ECGs, clinical laboratory tests, vital signs, and physical examinations
Immunogenicity measured by the confirmed presence of ADAs in plasma
Amyloid-related imaging abnormalities (ARIA-H and ARIA-E) and other emergent radiological findings

Secondary endpoints may include
Plasma PK of h2731
CSF PK of h2731
C_{obs} of h2731 at each sampling time

### Inclusion Criteria

Each cohort will contain approximately eight subjects with body mass indices (BMI) between 18.0 and 32.0 kg/m2.

AD subjects will be selected according to inclusion criteria that include the following:
(a) have a confirmed or suspected diagnosis of AD based on either probable AD with evidence of the AD pathophysiological process according to National Institute on Aging and Alzheimer's Association (NIA-AA) criteria (McKhann et al., Alzheimers Dement., 7(3):264-9, 2011) or high likelihood of AD according to NIA-AA criteria (Albert et al., Alzheimers Dement., 7(3):270-79, 2011);
(b) have gradual and progressive change in memory function for ≥6 months reported by subject or study partner;
(c) a screening Mini-Mental State Examination (MMSE) score ≥18; and
(d) evidence of AD pathological process, as confirmed on amyloid PET scan.

### Exclusion Criteria

Subjects will be selected based on exclusion criteria that include the following. Subjects must not meet any of the exclusion criteria, including the following criteria:
(a) Impaired coagulation (prothrombin time 1.2× ULN) or other coagulopathy
(b) History of severe, clinically significant (persistent neurologic deficit or structural brain damage) central nervous system (CNS) trauma (eg, cerebral contusion), epilepsy
(c) Have any contraindications for MRI studies, including claustrophobia, the presence of contraindicated metal (ferromagnetic) implants, or cardiac pacemaker
(d) Anti-coagulation medications within 3 months of screening with no plans to initiate any prior to randomization or history of prolonged bleeding after minor trauma. Note: low dose aspirin is permitted (up to 162 mg/day).
(e) History or presence of posterior reversible encephalopathy syndrome (PRES) (Fugate, et al, Posterior reversible encephalopathy syndrome: clinical and radiological manifestations, pathophysiology, and outstanding questions. Lancet Neurol. 2015; 14(9):914-25.)

### Clinical Laboratory Evaluations

Laboratory analysis of hematology, clinical chemistry, coagulation, urinalysis plasma, biomarkers, and CSF will be conducted. Central and local pregnancy testing will be conducted.

Brain MRI will be read locally and the scan will be submitted to the centralized MRI vendor for final determination of MRI eligibility, and to provide central assessment of baseline ARIA findings. MRIs should be performed using 1.5 or 3.0-T scanners, and the same scanner should be used for an individual subject for the duration of the study. The first MRI will occur during the screening period as a baseline measure to confirm structural brain imaging-based eligibility criteria. MRI scans will include, but are not limited to, the following sequences: T2-weighted FLAIR, 2-dimensional (2D) T2*-weighted gradient echo (GRE) or susceptibility weighted imaging (SWI). Diffusion weighted, 3-dimensional (3D) T1-weighted GRE. MRI scans will be evaluated and read by a MRI central reader, which will provide the diagnostic reads and assessments of MRI outcome measures. MRI data (Day 29 MRI data from all subjects and any other available MRI and safety data) will be made available for the DSMB for confirmation of h2731 dose.

CSF Analysis, if performed, in Cohorts will undergo 2 LPs: the first LP on Day 3 (48 hours after study drug administration) and the second LP on Day 29. The CSF will be analyzed to determine levels of h2731. CSF samples with clear evidence of blood contamination should not be used for PK assessment. CSF analyses will also include but are not limited to standard analyses including pressure, color, glucose, proteins, lactate, red blood cells, and white blood cells.

Amyloid PET Imaging will be used for biological confirmation of diagnosis of AD as evidence of presence of pathological hallmark findings of β-amyloid-composed neuritic plaques. Isotope-labelled compounds that show high affinity toward aggregated forms of β-amyloid (tracers) can provide evidence of β-amyloid in vivo. Three radioligand are being used for screening purposes: [18F]florbetapir/AV45 (Amyvid), [18F]flutemetamol (Vizamyl), and [18F]florbetaben (Neuraceq). Subjects in Cohorts 1-4 will undergo amyloid PET acquisition to confirm biologically the diagnosis of AD. A positive PET scan using [18F]florbetapir/AV45, [18F]flutemetamol, or [18F]florbetaben acquired outside of this trial protocol within 18 months prior to the first screening visit may be permissible to confirm patient inclusion with confirmation by a central read.

APOE4 status (eg, APOE4/APOE4, APOE4/APOE3, APOE3/APOE3, APOE4/APOE2, APOE3/APOE2) will be determined by central assessment for subjects enrolling into Cohorts 1 to 4.

### Additional Assessments

The Cogstate CBB (Maruff, 2013) will be administered to subjects at the first screening visit (Day−72 to −8). The CBB is a brief (approximately 15 minutes), computer-based cognitive test battery designed to measure memory, working memory psychomotor function, and attention. The CBB has been shown to be a sensitive tool for detecting AD-related cognitive decline in healthy older adults and in adults with amnestic mild cognitive impairment (Darby, 2002; Lim, 2013) as well as for improvement in cognition arising from treatment with cognition enhancing drugs (Davison, 2011; Jaeger, 2011; Nathan, 2013).

The MMSE (Folstein, 1975) will be administered to subjects at the first screening visit (Day−72 to −8), prior to the Cogstate CBB assessment, to determine if the subject meets entry criteria for cognitive impairment.

Subjects will undergo plasma sampling for Biomarkers of AD pathology including, but not limited to Aβ42/40, and biomarkers associated with tau pathology including, but not limited to, total tau, p181-tau, and p217-tau.

Subjects will undergo PK sampling for plasma and CSF h2731.

Plasma anti-h2731 antibody levels will be measured (antibodies detected with an electrochemiluminescent assay (ECLIA).

ARIA Assessment: a screening MRI scan will be used to exclude subjects with pre-existing vasogenic edema (ARIA-E), >4 microhemorrhages, or >1 area of superficial siderosis (ARIA H). In addition to scheduled MRIs, unscheduled MRIs may be obtained at the discretion of the Investigator upon suspicion of ARIA based on appearance of symptoms. MRIs will be scheduled prior to study drug administration (baseline) for all subjects and at the Day 29 and Day 85 visits (28- and 84-days post dose, respectively) for subjects with AD, and will be assessed, classified, and documented for radiographic evidence of ARIA.

### Example 20: Phase 1 Multiple Ascending Dose Study to Evaluate the Safety, Tolerability, Immunogenicity, Pharmacokinetics, and Pharmacodynamics of h2721 in Subjects with Alzheimer's Disease

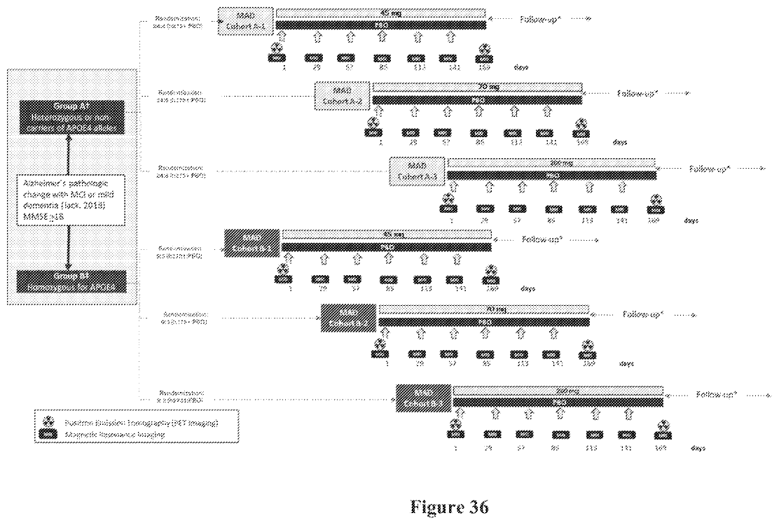

This example describes a phase 1, randomized, double-blind, placebo-controlled, multiple ascending dose (MAD) study to assess the safety, tolerability, and immunogenicity, PK, and pharmacodynamics (PD) effects of h2731 in patients with AD. FIG. 36 is a schematic representation of the study plan.

### Study Objectives

As discussed in further detail below, the primary objective of the study is to evaluate the safety, tolerability, and immunogenicity of h2731 after multiple SC doses. The secondary objectives of the study are to characterize the PK profile of h2731 after multiple SC doses, to characterize the plasma and CSF PK profile of h2731 after multiple SC doses, and to assess the PD effects of h2731 on brain amyloid plaque deposition after multiple SC doses. Exploratory objectives of the study are to assess PD effects of h2731 on blood and CSF biomarkers after multiple CS doses and to assess ARIA findings by apolipoprotein E4 (APOE4) status.

### Study Population

The study consists of two parts, each studying a different group of subjects: Group A, subjects with AD who are heterozygous or non-carriers of apolipoprotein E4 (APOE4) alleles (referred to as the non-homozygote population) and Group B, subjects with AD who are homozygous for APOE4 (referred to as the homozygote population). The dose levels of h2731 to be assessed will be the same for the non-homozygote (Group A) and homozygote (Group B) populations.

Apolipoprotein E (APOE) genotype status has been demonstrated to impact the onset of AD (Corder, 1993; van Duijn, 1994) as well as rates of ARIA following treatment with anti-Aβ antibodies (Arrighi, 2016; Ketter, 2017; Muralidharan, 2022). Development of ARIA is dose-dependent, and most events occur within the first few months after initiation of anti-Aβ treatment (Muralidharan, 2022). Patients with AD who possess 1 copy of the APOE4 allele may possess an elevated risk of anti-Aβ antibody-mediated ARIA compared to APOE4 non-carriers, although the risk demonstrated across clinical trials is somewhat inconsistent. However, patients who are APOE4 homozygous (possessing 2 alleles) consistently demonstrate higher incidents of ARIA compared to both APOE4 heterozygous and non-carrier patients.

### Rationale for Dose Selection

The proposed doses of 45 mg, 70 mg, 200 mg are based on analysis of estimated target engagement (fractional occupancy [fOcc]) levels resulting from simulated clinical drug exposure levels at these doses, balancing predictions of efficacious exposure and the potential for inducing ARIA. These doses are further supported by results from the completed nonclinical toxicity studies with repeated dose administration for up to 3 months, and will be confirmed based on the certain information from the single ascending dose (SAD) study of Example 19, including:
All safety and tolerability data through Day 15 from selected cohorts dosed at 70 mg and/or 200 mg;
PK data through Day 29 from selected cohorts dosed at 70 mg and/or 200 mg (approximately 8 subjects per cohort; 6 receiving h2731 and 2 receiving placebo); and
Day 29 MRI results from selected cohorts dosed at 70 mg and/or 200 mg (approximately 12 subjects; 9 receiving h2731 and 3 receiving placebo).

### Study Design

This Phase 1, randomized, double-blind, placebo-controlled, multiple ascending dose study will be conducted in two dose cohorts in subjects with biologically confirmed AD to assess the safety, tolerability, immunogenicity, PK, and PD of h2731.

The study consists of two parts, each evaluating a different group of subjects: Group A, subjects with AD who are heterozygous or non-carriers of APOE4 alleles and Group B, subjects with AD who are homozygous for APOE4. The three dose cohorts for each group are described in Table 21.

TABLE 21

| Group A: | |
|---|---|
| APOE4 heterozygous (eg, E3/E4) or | Group B: |
| APOE4 non-carrier (eg, E2/E3, E2/E2) | APOE4 homozygous (ie, E4/E4) |
| Cohort A-2: 45 mg | Cohort B-2: 45 mg |
| Cohort A-2: 70 mg | Cohort B-2: 70 mg |
| Cohort A-3: 200 mg | Cohort B-3: 200 mg |

Subjects with AD who are APOE4 heterozygous or non-carriers of APOE4 alleles will be assigned to Group A: subjects who are homozygous for APOE4 will be assigned to Group B.

For each Group A cohort, approximately 32 subjects will be randomly assigned to h2731 or placebo in a 3:1 ratio: approximately 24 subjects will receive h2731, and approximately 8 subjects will receive placebo. Randomization will be stratified by APOE4 carrier status (APOE4 heterozygous or APOE4 non-carrier).

For each Group B cohort, approximately 12 subjects will be randomly assigned to h2731 or placebo in a 3:1 ratio: approximately 9 subjects will receive h2731, and approximately 3 subjects will receive placebo.

### Treatment Period

Study drug (45 mg, 70 mg, or 200 mg) will be administered every 4 weeks starting on Day 1 for a total of up to 6 doses. Subjects will receive the first dose of study drug (h2731 or placebo) administered subcutaneously on Day 1. Subjects will undergo safety assessments including adverse event (AE) monitoring, clinical laboratory tests, vital signs, physical examinations, and electrocardiograms (ECGs), as well as blood collections for PK, anti-drug antibody (ADA), and biomarker (BM) analysis. Subjects will be released from the study site 8 hours after dosing, after completing scheduled post-dose assessments.

Dosing will continue every 4 weeks. Safety assessments and blood collections for PK, ADA, and BM analysis will be completed. MRI findings, as assessed by the central reader, must be reviewed prior to dosing to evaluate for the presence of ARIA.

After the last administration of study drug, subjects will return to the study site for the Week 24 (Day 169) visit to complete an end of treatment (EOT) visit that includes safety assessments, blood collections for PK, ADA, and BM analysis, and amyloid PET imaging assessment.

Subjects who participate in the optional CSF collection will be scheduled for CSF collection to occur within 1 to 5 days after the Week 24 (Day 169) imaging visit (MRI and PET). Subjects may be released after 4 hours of observation.

### Dose Escalation/Dose Determination

The dose levels of each cohort will be determined by a limited number of unblinded Sponsor representatives based on review and interpretation of all available safety, tolerability, PD, and PK information for h2731.

Safety and tolerability data will be evaluated in an ongoing and periodic manner throughout this study and SAD study of Example 19. Safety and tolerability data will be evaluated to provide a recommendation on the decision to enroll cohorts when the minimum data requirements have been met.

### Suspension of Dosing on Subject Level

Dosing depends on the presence and severity of amyloid-related imaging findings related to underlying vasogenic edema (ARIA-E) or hemorrhage (ARIA-H) observed prior to each administration of h2731 or placebo based on MRI findings, as assessed by the MRI central reader, and potential symptoms of ARIA reported by the subject or observed by the Investigator.

An MRI visit will be scheduled up to 7 days before each dosing visit. ARIA severity classifications based on radiographic findings are summarized in Table 22. MRI results must be reviewed prior to dosing.

TABLE 22

| ARIA Type | Mild | Mild+ | Moderate | Moderate+ | Severe |
|---|---|---|---|---|---|
| ARIA-E | FLAIR hyper- | FLAIR hyper- | FLAIR hyper- | FLAIR hyper- | FLAIR hyperintensity |
| | instensity | instensity | instensity in | instensity >1 | measuring >10 cm, |
| | confined to | cortex/ | 1 location; | location; | often with significant |
| | sulcus or | subcortical | Extent of | Extent of | subcortical white matter |
| | cortex / | white matter | 5-10 cm | 5-10 cm each | and/or sulcal |
| | subcortical | in >1 location; | | | involvement. >1 |
| | white matter | Extent of <5 cm | | | separate site of |
| | in 1 location; | | | | involvement may be |
| | Extent of <5 cm | | | | noted |
| | Mild | | Moderate | | Severe |
| ARIA-H/ | ≤4 new incidents | | 5-9 new incidents | | ≥10 new incidents |
| micro- | microhemorrhages^{a} | | microhemorrhages^{a} | | microhemorrhages^{a} |
| hemorrhage | | | | | |
| ARIA-H | 1 focal area of | | 2 focal areas of | | >2 focal areas of |
| Superficial | superficial siderosis | | superficial siderosis | | superficial siderosis |
| siderosis | | | | | |

ARIA-E = amyloid-related imaging findings related to underlying vasogenic edema;
ARIA-H = amyloid-related imaging findings related to intracerebral hemorrhage;
FLAIR = fluid-attenuated inversion recovery
^{a}New microhemorrhage counted cumulatively compared to screening visit.
Source: adapted from Aduhelm USPI, 2021 and Bracoud, 2017

All intracerebral hemorrhage greater than 1 cm are considered radiographically severe.

Dosing may be suspended based on a moderate and/or severe ARIA-E finding based on either ARIA-E Clinical Severity or ARIA-E Radiographic Severity. Any new ARIA-H (microhemorrhage or superficial siderosis) finding, except for asymptomatic mild ARIA-H, leads to suspension of dosing of h2731 for the subject.

If dosing is suspended, the scheduled dose should be completely omitted, and subsequent visits should be continued as scheduled. When the subject is able to re-initiate treatment (Section 7.11.3), administration of h2731 or placebo should resume at the same dose at the time of the next scheduled dose. Omitted doses will not be replaced.

### Endpoints

Primary endpoints will include:
Safety and tolerability based on AE reporting (incidence of AEs, SAEs, and H2731-related AEs), vital signs, physical and neurological examinations, 12-lead ECGs, clinical laboratory tests
Injection site reactions based on Investigator assessment
Nature, frequency, severity, and timing of MRI findings of ARIA, including incidence of symptomatic ARIA-E and/or ARIA-H; incidence of isolated ARIA-E (only ARIA-E, no ARIA-H), and isolated ARIA-H (only ARIA-H, no ARIA-E); and incidence of concurrent ARIA-E and ARIA-H
Presence of ADAs in plasma

Secondary endpoints will include:
Plasma PK of h2731
Maximum observed concentration (Cₘₐₓ)
Time of the maximum measured concentration (Tmax)
Last concentration time point before the next administration (Ctrough)
Area under the concentration-time curve from time zero to infinity (AUC0-∞)
Area under the plasma concentration-time curve for dosing interval (AUC0-tau)
Area under the plasma concentration-time curve accumulation ratio over one dosing interval from the first to last dose (RAUC)
Apparent volume of distribution (Vd)
Average concentration over the dosing interval (Cavg)
Apparent total body clearance (CL/F)
CSF PK of h2731 (optional CSF collection)
   Observed concentration (Cobs) of h2731 in plasma and CSF at each sampling time of CSF collection
Change from baseline in brain amyloid plaque deposition as measured by amyloid positron emission tomography (PET) scan at Week 24 (Day 169)

Exploratory endpoints will include:
Change in blood-based biomarkers from baseline through Week 24 (Day 169) including but not limited to Aβ42/40 ratio, p181-tau, and p217-tau
Change in CSF biomarkers from baseline through Week 24 (Day 169) including but not limited to Aß42/40 ratio, p181-tau, and p217-tau (for subjects in optional CSF collection)
Nature, frequency, severity, and timing of MRI findings by APOE4 status (APOE4 heterozygous or APOE4 non-carrier), including incidence of symptomatic ARIA-E and/or ARIA-H; incidence of isolated ARIA-E (only ARIA-E, no ARIA-H), and isolated ARIA-H (only ARIA-H, no ARIA-E); and incidence of concurrent ARIA-E and ARIA-H

### Inclusion Criteria

AD subjects will be selected according to inclusion criteria that include the following:
Subjects between 55 and 85 years having a body mass index between 18.0 and 32.0 kg/m2, inclusive;
Gradual and progressive change in memory function for ≥6 months reported by subjects or study partner;
Alzheimer's pathologic change with either mild cognitive impairment or mild dementia (Stage 2, 3 or 4) according to National Institute on Aging and Alzheimer's Association (NIA-AA) criteria (Jack, 2018; Appendix 3); and
Screening MMSE score ≥18.

### Exclusion Criteria

Exclusion Criteria are similar to Example 19, with the additional exclusion of family history of dominantly inherited AD
Clinical laboratory evaluations, Cogstate assessment, MMSE assessment, PK sampling, biomarker analysis, and ARIA assessment will be performed using methods similar to those set forth in Example 19.

### Results

The results of this MAD study will show a reduction in amyloid plaque in treated patients (e.g., amyloid reduction described in the present disclosure) with relatively low ARIA rates (e.g., ARIA rates and ARIA risks described in the present disclosure).

### Example 21: Phase 1 Open Label Extension Study to Evaluate the Safety, Tolerability, Immunogenicity, Pharmacokinetics, and Pharmacodynamics of h2721 in Subjects with Alzheimer's Disease

This example describes an open-label extension (OLE) study for subjects with Alzheimer's disease who participated in and completed the single ascending dose study (SAD) of Example 19 or participated in and completed the treatment period of the multiple ascending dose study (MAD) of Example 20 and meet eligibility criteria for this OLE as described further herein.

### Study Design

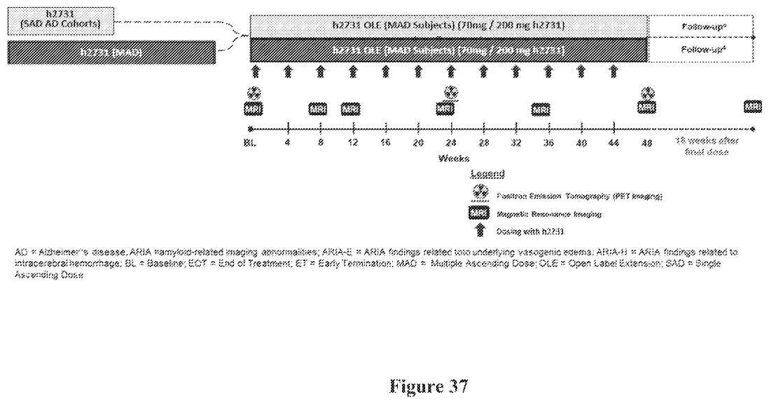

All subjects will receive up to 12 doses of h2731 in this OLE as summarized in FIG. 37. h2731 will be administered once every 4 weeks by subcutaneous injection for a total of up to 12 doses. All subjects from the SAD study will receive 70 mg h2731 and subjects from the MAD study will receive a dose of h2731 based on their cohort assignment (Cohorts A-1, A-2, A-3, B-1, B-2, or B-3) in the core study.

### Study Objectives

The primary objective of the OLE is to evaluate long-term safety, tolerability, and immunogenicity of h2731. The secondary objective of the OLE is to characterize PK profile of h2731. Exploratory objectives include assessing the effect of h2731 on amyloid positron emission tomography (PET), and assessing the PD effects of h2731 on plasma biomarkers.

### Eligibility Criteria

For subjects from the SAD study of Example 19, screening and enrollment into this OLE can occur after completion of a Day 85 visit.

For subjects from the MAD study of Example 20, screening and enrollment into this OLE must occur no more than 18 weeks (127 days) after Week 24/End of Treatment (EOT) and will be based on the following:

If no ARIA at the Week 24/EOT in the study SAD study of Example 19, subjects can enter this OLE if all eligibility criteria are met and no more than 18 weeks have elapsed since the Week 24/EOT visit and first dose for the OLE study. Eligible subjects will be encouraged to be dosed within 2 weeks of the Week 24/EOT visit for a more seamless transition.

If ARIA (either new or ongoing) that does not require dose suspension is detected at the Week 24/EOT visit from the MAD study of Example 20, subjects can enter the OLE study if all eligibility criteria are met and no more than 18 weeks have elapsed since the Week 24/EOT visit and first dose of the OLE.

If ARIA (either new or ongoing) that requires dose suspension is detected at the Week 24/EOT visit of MAD study, subjects can enter this OLE if (1) the ARIA finding has stabilized (for ARIA-H) or resolved (for ARIA-E) and (2) no more than 18 weeks have elapsed since the Week 24/EOT visit of the MAD study and first dose of the OLE and (3) it is acceptable to resume dosing with h2731 in the opinion of the Investigator as guided by clinical judgment, and (4) all eligibility criteria are met.

If ARIA has not stabilized or resolved after 18 weeks (127 days) have elapsed since the Week 24/EOT visit of the MAD study, the subject is not eligible to enroll in OLE and will complete follow-up in the MAD study.

### Endpoints

Primary endpoints will include:
Safety and tolerability based on AE reporting (incidence of adverse events [AEs], serious adverse events [SAEs], and h2731-related AEs), clinical laboratory tests, vital signs, and physical examinations;
Nature, frequency, severity, and timing of magnetic resonance imaging (MRI) findings of ARIA, including amyloid-related imaging findings related to underlying vasogenic edema (ARIA E); amyloid-related imaging findings related to intracerebral hemorrhage (ARIA-H); incidence of symptomatic ARIA-E and/or ARIA-H; and incidence of concurrent ARIA-E and ARIA-H
Presence of anti-drug antibodies (ADAs) in plasma

Secondary Endpoints will include plasma concentration of h2731.

Exploratory Endpoints will include:
brain amyloid levels as measured by PET imaging; and
plasma-based biomarkers including but not limited to Aß42, Aß40, amyloid beta peptide ratio 42/40 (Aβ42/40), p181-tau, p217-tau.

Additional analyses may be performed to evaluate brain amyloid centiloid change from the core study baseline after 24 and 48 weeks of treatment in the OKE. For this analysis, all timepoints are presented relative to start of h2731 exposure. Percentage (%) of subjects who reach amyloid negativity based on amyloid PET scan at Week 24 (Day 169) and Week 48 (Day 337) will also be presented.

The PK/PD relationship between centiloid reduction and cumulative dose of h2731 may be summarized. Additional analysis may be performed to examine the PK/PD relationship between centiloid reduction and h2731 plasma exposure.

Inclusion Criteria include completion of the SAD study of Example 19 (AD cohorts only) or completed the treatment period in the MAD study of Example 20, are those as in Example 20.

Exclusion criteria include those listed in Example 20, including ARIA that would prohibit dosing as shown in Tables 24 and 25 from Example 20.

### Results

The results of this OLE study will show a reduction in amyloid plaque in treated patients (e.g., amyloid reduction described in the present disclosure) with relatively low ARIA rates (e.g., ARIA rates and ARIA risks described in the present disclosure).

All publications (including GenBank Accession numbers, UniProtKB/Swiss-Prot accession numbers and the like), patents and patent applications cited are herein incorporated by reference in their entirety for all purposes to the same extent as if each individual publication, patent and patent application was specifically and individually indicated to be incorporated by reference in its entirety for all purposes. In the event of any variance in sequences associated with Genbank and UniProtKB/Swiss-Prot accession numbers and the like, the application refers to the sequences associated with the cited accession numbers as of the effective filing date of the application meaning the actual filing date or earlier date of a priority application disclosing the relevant accession number. Any feature, step, element, embodiment, or aspect of the disclosure can be used in combination with any other unless specifically indicated otherwise. Although the present disclosure has been described in some detail by way of illustration and example for purposes of clarity and understanding, it will be apparent that certain changes and modifications may be practiced within the scope of the appended claims.

1-163. (canceled)

164. An anti-amyloid β antibody or antigen-binding fragment thereof for use in a method of treating Alzheimer's disease or reducing amyloid plaque in a subject or converting a subject from amyloid positive to amyloid negative, comprising administering to the subject about 20 mg to about 200 mg of the anti-amyloid β antibody or an antigen-binding fragment thereof once about every 3-5 weeks, wherein the anti-amyloid β antibody or antigen-binding fragment thereof comprises heavy chain CDR1 comprises the amino acid sequence of SEQ ID NO: 16,
heavy chain CDR2 comprises the amino acid sequence of SEQ ID NO: 20,
heavy chain CDR3 comprises the amino acid sequence of SEQ ID NO: 18,
light chain CDR1 comprises the amino acid sequence of SEQ ID NO: 29,
light chain CDR2 comprises the amino acid sequence of SEQ ID NO: 34, and
light chain CDR3 comprises the amino acid sequence of SEQ ID NO: 38.

165. The anti-amyloid β antibody or antigen-binding fragment thereof for use according to claim 164, wherein the method comprises administering about 20 mg to about 100 mg of the anti-amyloid β antibody or antigen-binding fragment thereof, or
about 100 mg to about 200 mg of the anti-amyloid β antibody or antigen-binding fragment thereof, or
about 45 mg of the anti-amyloid β antibody or antigen-binding fragment thereof, or
about 70 mg of the anti-amyloid β antibody or antigen-binding fragment thereof, or
about 200 mg of the anti-amyloid β antibody or antigen-binding fragment thereof.

166. The anti-amyloid β antibody or antigen-binding fragment thereof for use according to claim 164, wherein the anti-amyloid β antibody or antigen-binding fragment thereof is administered as a pharmaceutical composition comprising the anti-amyloid β antibody or antigen-binding fragment thereof and a pharmaceutically acceptable excipient.

167. The anti-amyloid β antibody or antigen-binding fragment thereof for use according to claim 164, wherein the administration is intravenous or subcutaneous.

168. The anti-amyloid β antibody or antigen-binding fragment thereof for use according to claim 164, wherein the heavy chain variable region, excluding the CDRs, is at least 98% identical to the amino acid sequence of SEQ ID NO: 3, and the light chain variable region, excluding the CDRs, is at least 98% identical to the amino acid sequence of SEQ ID NO: 9.

169. The anti-amyloid β antibody or antigen-binding fragment thereof for use according to claim 164, wherein the anti-amyloid β antibody is a humanized IgG1, a chimeric antibody, a CDR-grafted antibody, or a recombinant antibody.

170. The anti-amyloid β antibody or antigen-binding fragment thereof for use according to claim 164, wherein the anti-amyloid β antibody or antigen-binding fragment thereof further comprises a heavy chain constant region comprising an amino acid sequence at least 98% identical to SEQ ID NO: 40 and/or a light chain constant region comprising an amino acid sequence at least 98% identical to SEQ ID NO: 41, or
wherein the anti-amyloid β antibody comprises a heavy chain of SEQ ID NO: 101, with or without the C-terminal lysine, and a light chain of SEQ ID NO: 102.

171. The anti-amyloid β antibody or antigen-binding fragment thereof for use according to claim 164, wherein, compared to a baseline,
amyloid plaque in the subject is reduced, and/or
the ratio of Aβ 42/40 is increased in the subject, and/or
an amount of phospho-tau is decreased in the subject, wherein the phosphor-tau is one or more of p181-tau, a p212-tau, p217-tau, a p231-tau, and a p235-tau.

172. The anti-amyloid β antibody or antigen-binding fragment thereof for use according to claim 164, wherein the duration of the treatment is at least 6 months.

173. A pharmaceutical composition in a dosage form, the pharmaceutical composition comprising:
a) an anti-amyloid β antibody or antigen-binding fragment thereof comprising a heavy chain variable region comprising heavy chain CDR1, CDR2, and CDR3 and a light chain variable region comprising light chain CDR1, CDR2, and CDR3, wherein
heavy chain CDR1 comprises the amino acid sequence of SEQ ID NO: 16,
heavy chain CDR2 comprises the amino acid sequence of SEQ ID NO: 20,
heavy chain CDR3 comprises the amino acid sequence of SEQ ID NO: 18,
light chain CDR1 comprises the amino acid sequence of SEQ ID NO: 29,
light chain CDR2 comprises the amino acid sequence of SEQ ID NO: 34, and
light chain CDR3 comprises the amino acid sequence of SEQ ID NO: 38; and
b) a pharmaceutically acceptable excipient,
wherein the dosage form delivers from 20 mg to 200 mg of the anti-amyloid β antibody or antigen-binding fragment thereof to a subject when the pharmaceutical composition is delivered to the subject.

174. The pharmaceutical composition of claim 173, wherein the heavy chain variable region, excluding the CDRs, is at least 98% identical to the amino acid sequence of SEQ ID NO: 3, and the light chain variable region, excluding the CDRs, is at least 98% identical to the amino acid sequence of SEQ ID NO: 9.

175. The pharmaceutical composition of claim 173, wherein the anti-amyloid β antibody is a humanized IgG1, a chimeric antibody, a CDR-grafted antibody, or a recombinant antibody.

176. The pharmaceutical composition of claim 174, wherein the anti-amyloid β antibody is a humanized IgG1, a chimeric antibody, a CDR-grafted antibody, or a recombinant antibody.

177. The pharmaceutical composition of claim 173, wherein the anti-amyloid β antibody or antigen-binding fragment thereof further comprises a heavy chain constant region comprising an amino acid sequence at least 98% identical to SEQ ID NO: 40 and/or a light chain constant region comprising an amino acid sequence at least 98% identical to SEQ ID NO: 41, or
wherein the anti-amyloid β antibody comprises a heavy chain of SEQ ID NO: 101, with or without the C-terminal lysine, and a light chain of SEQ ID NO: 102.

178. The pharmaceutical composition of claim 174, wherein the anti-amyloid β antibody or antigen-binding fragment thereof further comprises a heavy chain constant region comprising an amino acid sequence at least 98% identical to SEQ ID NO: 40 and/or a light chain constant region comprising an amino acid sequence at least 98% identical to SEQ ID NO: 41, or
wherein the anti-amyloid β antibody comprises a heavy chain of SEQ ID NO: 101, with or without the C-terminal lysine, and a light chain of SEQ ID NO: 102.

179. The pharmaceutical composition of claim 175, wherein the anti-amyloid β antibody or antigen-binding fragment thereof further comprises a heavy chain constant region comprising an amino acid sequence at least 98% identical to SEQ ID NO: 40 and/or a light chain constant region comprising an amino acid sequence at least 98% identical to SEQ ID NO: 41, or
wherein the anti-amyloid β antibody comprises a heavy chain of SEQ ID NO: 101, with or without the C-terminal lysine, and a light chain of SEQ ID NO: 102.

180. The pharmaceutical composition of claim 176, wherein the anti-amyloid β antibody or antigen-binding fragment thereof further comprises a heavy chain constant region comprising an amino acid sequence at least 98% identical to SEQ ID NO: 40 and/or a light chain constant region comprising an amino acid sequence at least 98% identical to SEQ ID NO: 41, or
wherein the anti-amyloid β antibody comprises a heavy chain of SEQ ID NO: 101, with or without the C-terminal lysine, and a light chain of SEQ ID NO: 102.

181. The pharmaceutical composition of claim 173, wherein the dosage form delivers from 100 mg to 200 mg of the anti-amyloid β antibody or antigen-binding fragment thereof to a subject when the pharmaceutical composition is delivered to the subject, or
wherein the dosage form delivers 45 mg of the anti-amyloid β antibody or antigen-binding fragment thereof to a subject when the pharmaceutical composition is delivered to the subject, or
wherein the dosage form delivers 70 mg of the anti-amyloid β antibody or antigen-binding fragment thereof to a subject when the pharmaceutical composition is delivered to the subject, or
wherein the dosage form delivers 200 mg of the anti-amyloid β antibody or antigen-binding fragment thereof to a subject when the pharmaceutical composition is delivered to the subject.

182. The pharmaceutical composition of claim 173, wherein the dosage form is formulated as a subcutaneous injection or intravenous injection.

183. A syringe or an autoinjector comprising the pharmaceutical composition of claim 173.


SEQUENCE LISTING (104 sequences)

SEQ ID NO: 1-2 - skipped (2 entries)

SEQ ID NO: 3 - protein, 119 aa, synthetic construct
  REGION 1..119: note = Synthetic peptide
EVQLLESGGGLVQPGGSLRLSCAASGFTFSNYGMSWVRQAPGKGLEWVASIRSGSGRTYYSDNVKGRFTISRDNSKNTLYLQMNSLRAEDTAVYYCVRYDHYSGSSDYWGQGTLVTVSS

SEQ ID NO: 4 - protein, 119 aa, synthetic construct
  REGION 1..119: note = Synthetic peptide
EVQLLESGGGLVQPGGSLRLSCAASGFTFSNFGMSWVRQAPGKGLEWVASVRSGSGRTYYSDNVKGRFTISRDNSKNTLYLQMNSLRAEDTAVYYCVRYDHYSGTSDYWGQGTLVTVSS

SEQ ID NO: 5 - protein, 119 aa, synthetic construct
  REGION 1..119: note = Synthetic peptide
EVQLLESGGGLVQPGGSLRLSCAASGFTFSNFGMSWVRQAPGKGLEWVASVRSGSGRTYYSDNVKGRFTISRDNSKNTLYLQMNSLRAEDTAVYYCVRYDHYTGTSDYWGQGTLVTVSS

SEQ ID NO: 6 - protein, 119 aa, synthetic construct
  REGION 1..119: note = Synthetic peptide
EVQLLESGGGLVQPGGSLRLSCAASGFTFSNFGMSWVRQAPGKGLEWVASVRSGGGRTYYSDNVKGRFTISRDNSKNTLYLQMNSLRAEDTAVYYCVRYDHYSGTSDYWGQGTLVTVSS

SEQ ID NO: 7 - protein, 119 aa, synthetic construct
  REGION 1..119: note = Synthetic peptide
EVQLLESGGGLVQPGGSLRLSCAASGFTFANYGMSWVRQAPGKGLEWVASVRSGGSRTYYSDNVKGRFTISRDNSKNTLYLQMNSLRAEDTAVYYCVRYDHYSGSSDYWGQGTLVTVSS

SEQ ID NO: 8 - protein, 112 aa, synthetic construct
  REGION 1..112: note = Synthetic peptide
DVVMTQSPLSLPVTPGEPASISCKSSQSLLDYDGKTYLNWLLQKPGQSPQRLIYKVSNRDSGVPDRFSGSGSGTDFTLKISRVEAEDVGVYYCWQGTHFPRTFGQGTKVEIK

SEQ ID NO: 9 - protein, 112 aa, synthetic construct
  REGION 1..112: note = Synthetic peptide
DVVMTQSPLSLPVTLGEPASISCKSSQSLLDYDGKTYLNWLLQKPGQSPQRLIYRVTNRDTGVPDRFSGSGSGTDFTLKISRVEAEDVGVYYCWQGTHFPRSFGQGTKVEIK

SEQ ID NO: 10 - protein, 112 aa, synthetic construct
  REGION 1..112: note = Synthetic peptide
DVVMTQSPLSLPVTLGEPASISCKSSQSLLDSDGKTYLNWLLQKPGQSPQRLIYRVTNRDTGVPDRFSGSGSGTDFTLKISRVEAEDVGVYYCWQGTHFPRTFGQGTKVEIK

SEQ ID NO: 11 - protein, 112 aa, synthetic construct
  REGION 1..112: note = Synthetic peptide
DVVMTQSPLSLPVTPGEPASISCRSSQSLVDYDGKTYLNWLLQRPGQSPQRLIYKVSNRDSGVPDRFSGSGSGTDFTLKISRVEAEDVGVYYCWQGSHFPRSYGQGTKVEIK

SEQ ID NO: 12 - protein, 112 aa, synthetic construct
  REGION 1..112: note = Synthetic peptide
DVVMTQSPLSLPVTLGEPASISCKSSQSLMDTDGKTYLNWLLQKPGQSPQRLIYKVSNRESGVPDRFSGSGSGTDFTLKISRVEAEDVGVYYCWQGTHFPRTFGQGTKVEIK

SEQ ID NO: 13 - protein, 112 aa, synthetic construct
  REGION 1..112: note = Synthetic peptide
DVVMTQSPLSLPVTPGEPASISCKSSQSLLDYDGKTYLNWLLQKPGQSPQRLIYKVSNRDSGVPDRFSGSGSGTDFTLKISRVEAEDVGVYYCWQGTHFPRSFGQGTKVEIK

SEQ ID NO: 14 - protein, 112 aa, synthetic construct
  REGION 1..112: note = Synthetic peptide
DVVMTQSPLSLPVTLGEPASISCKSSQSLLDSDGKTYLNWLLQKPGQSPQRLIYKVSNRDSGVPDRFSGSGSGTDFTLKISRVEAEDVGVYYCWQGTHFPRTFGQGTKVEIK

SEQ ID NO: 15 - protein, 112 aa, synthetic construct
  REGION 1..112: note = Synthetic peptide
DVVMTQSPLSLPVTLGEPASISCKSSQSLLDSDGKTYLNWLLQKPGQSPQRLIYKVTNRESGVPDRFSGSGSGTDFTLKISRVEAEDVGVYYCWQGTHFPRSFGQGTKVEIK

SEQ ID NO: 16 - protein, 10 aa, synthetic construct
  REGION 1..10: note = Synthetic peptide
GFTFSNYGMS

SEQ ID NO: 17 - protein, 19 aa, synthetic construct
  REGION 1..19: note = Synthetic peptide
SIRSGGGRTYYSNDYNVKG

SEQ ID NO: 18 - protein, 10 aa, synthetic construct
  REGION 1..10: note = Synthetic peptide
YDHYSGSSDY

SEQ ID NO: 19 - protein, 10 aa, synthetic construct
  REGION 1..10: note = Synthetic peptide
GFTFSNFGMS

SEQ ID NO: 20 - protein, 17 aa, synthetic construct
  REGION 1..17: note = Synthetic peptide
SIRSGSGRTYYSDNVKG

SEQ ID NO: 21 - protein, 17 aa, synthetic construct
  REGION 1..17: note = Synthetic peptide
SVRSGSGRTYYSDNVKG

SEQ ID NO: 22 - protein, 17 aa, synthetic construct
  REGION 1..17: note = Synthetic peptide
SVRSGGGRTYYSDNVKG

SEQ ID NO: 23 - protein, 17 aa, synthetic construct
  REGION 1..17: note = Synthetic peptide
SVRSGGSRTYYSDNVKG

SEQ ID NO: 24 - protein, 10 aa, synthetic construct
  REGION 1..10: note = Synthetic peptide
YDHYSGTSDY

SEQ ID NO: 25 - protein, 10 aa, synthetic construct
  REGION 1..10: note = Synthetic peptide
YDHYTGTSDY

SEQ ID NO: 26 - protein, 16 aa, synthetic construct
  REGION 1..16: note = Synthetic peptide
KSSQSLLDSDGKTYLN

SEQ ID NO: 27 - protein, 7 aa, synthetic construct
LVSKLDS

SEQ ID NO: 28 - protein, 9 aa, synthetic construct
  REGION 1..9: note = Synthetic peptide
WQGTHFPRT

SEQ ID NO: 29 - protein, 16 aa, synthetic construct
  REGION 1..16: note = Synthetic peptide
KSSQSLLDYDGKTYLN

SEQ ID NO: 30 - skipped

SEQ ID NO: 31 - protein, 16 aa, synthetic construct
  REGION 1..16: note = Synthetic peptide
RSSQSLVDYDGKTYLN

SEQ ID NO: 32 - protein, 16 aa, synthetic construct
  REGION 1..16: note = Synthetic peptide
KSSQSLMDTDGKTYLN

SEQ ID NO: 33 - protein, 7 aa, synthetic construct
  REGION 1..7: note = Synthetic peptide
KVSNRDS

SEQ ID NO: 34 - protein, 7 aa, synthetic construct
  REGION 1..7: note = Synthetic peptide
RVTNRDT

SEQ ID NO: 35 - protein, 7 aa, synthetic construct
  REGION 1..7: note = Synthetic peptide
KVSNRES

SEQ ID NO: 36 - protein, 7 aa, synthetic construct
  REGION 1..7: note = Synthetic peptide
KVTNRES

SEQ ID NO: 37 - skipped

SEQ ID NO: 38 - protein, 9 aa, synthetic construct
  REGION 1..9: note = Synthetic peptide
WQGTHFPRS

SEQ ID NO: 39 - protein, 9 aa, synthetic construct
  REGION 1..9: note = Synthetic peptide
WQGSHFPRS

SEQ ID NO: 40 - protein, 330 aa, synthetic construct
  REGION 1..330: note = Synthetic peptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 107 aa, synthetic construct [sequence omitted]

SEQ ID NO: 42 - DNA, 357 bp, synthetic construct
  source 1..357: mol_type = other DNA
gaagtgcagcttctggagagcgggggcggcctggtgcagccgggcggatccctgagactgtcctgtgccgcgtccggttttaccttctccaactacggaatgtcatgggtccgccaagcacccggaaagggattggaatgggtggcttcgatccggtccggctcgggacggacctactactccgataacgtcaagggcagattcactattagccgggacaacagcaagaataccctgtacctccaaatgaactccctgagggccgaggacaccgccgtgtattactgcgtgcgctacgaccactactcgggttcctctgattactggggacaggggaccctcgtgactgtgtcaagc

SEQ ID NO: 43 - DNA, 336 bp, synthetic construct
  source 1..336: mol_type = other DNA
gatgtcgtgatgacccagtcaccactgtcccttcctgtgactcccggagaaccggcgtccatttcgtgcaagagcagccagtccctgctcgattatgacggaaagacctacctgaactggttgctccaaaagcctgggcagagcccccagagactgatctacaaagtgtccaacagggactcgggcgtgccggaccgcttctcggggtccggttccggtaccgactttacgctgaagatctcacgggtggaagccgaggacgtgggagtgtactactgttggcagggcactcacttcccgcggaccttcggacaaggcaccaaggtcgagatcaag

SEQ ID NO: 44 - DNA, 357 bp, synthetic construct
  source 1..357: mol_type = other DNA
gaagtgcagctcctggagtccgggggtggactggtgcagcccgggggcagcctgaggctgagctgcgccgcgtcaggattcaccttctccaacttcggaatgtcctgggtcagacaggccccgggaaagggccttgaatgggtggctagcgtgcgctccggttccggacggacctactactcggacaacgtgaagggccggtttactatctcccgggacaattcgaagaacaccctgtacctccaaatgaactccttgcgcgccgaggataccgcagtgtattactgcgtgcgctacgaccactactctggcactagcgattactggggccagggaactctggtcaccgtgtcgtca

SEQ ID NO: 45 - DNA, 336 bp, synthetic construct
  source 1..336: mol_type = other DNA
gatgtcgtgatgactcagtcacctctgtccctgcctgtgacccttggggaacccgcctcgatctcgtgcaagagctcccagagcctgctcgactatgatggaaagacctacctgaactggttgctccaaaagccgggccagagcccccagaggctgatctaccgcgtgaccaaccgcgacaccggggtgccggaccggttctccggatccggcagcggcactgacttcaccctgaaaatttccagagtggaagccgaggacgtgggagtgtactactgttggcagggtactcactttccacggtccttcggtcaaggaaccaaggtcgagatcaag

SEQ ID NO: 46 - DNA, 357 bp, synthetic construct; identical to SEQ ID NO: 42
  source 1..357: mol_type = other DNA

SEQ ID NO: 47 - DNA, 336 bp, synthetic construct; identical to SEQ ID NO: 45
  source 1..336: mol_type = other DNA

SEQ ID NO: 48 - DNA, 357 bp, synthetic construct
  source 1..357: mol_type = other DNA
gaagtgcagctgctggagtctggcggcggactggtgcagcccgggggatccctgcggctttcctgcgccgcatccggcttcaccttttcaaacttcggaatgtcgtgggtcagacaggccccgggaaagggtctggaatgggtggcctcagtgcggtccggatcgggtagaacctactacagcgataacgtgaagggccggttcacgatctcccgcgacaactccaagaacaccctgtacttgcaaatgaatagcctcagggctgaggataccgcggtctactactgtgtgcgctatgaccactacactggaactagcgactactggggccaggggaccctcgtgactgtgtcgtcc

SEQ ID NO: 49 - DNA, 336 bp, synthetic construct; identical to SEQ ID NO: 45
  source 1..336: mol_type = other DNA

SEQ ID NO: 50 - DNA, 357 bp, synthetic construct; identical to SEQ ID NO: 44
  source 1..357: mol_type = other DNA

SEQ ID NO: 51 - DNA, 336 bp, synthetic construct; identical to SEQ ID NO: 43
  source 1..336: mol_type = other DNA

SEQ ID NO: 52 - DNA, 357 bp, synthetic construct
  source 1..357: mol_type = other DNA
gaggtgcagctgctggagtcgggggggggactcgtgcagcccgggggctccctgagactctcttgtgccgcctccggcttcactttttcaaacttcggaatgtcctgggtccgccaagcaccgggaaagggtctggaatgggtcgccagcgtgcggtccggcggcggacggacttactactccgacaacgtgaagggccggttcaccatctcaagggataactccaagaatactctgtacttgcaaatgaactcgctgcgcgctgaagataccgcggtgtactattgcgtgcgctacgaccactactccggtaccagcgactactggggacagggaacccttgtgaccgtgtcgagc

SEQ ID NO: 53 - DNA, 336 bp, synthetic construct
  source 1..336: mol_type = other DNA
gatgtcgtgatgactcagtcgcccctctccctgcctgtgactctgggggaacccgcgtccatttcgtgcaagagcagccagtccctgttggactcagacggaaagacctaccttaactggctgctgcaaaagccaggacagagcccgcagaggctgatctaccgcgtgaccaaccgggatacgggagtgccggacagattcagcggctcgggttccggcaccgacttcaccctcaaaatctcccgcgtcgaggccgaggacgtgggcgtgtattactgttggcagggaacccactttcctcggaccttcggtcaagggactaaggtcgaaatcaag

SEQ ID NO: 54 - DNA, 357 bp, synthetic construct; identical to SEQ ID NO: 48
  source 1..357: mol_type = other DNA

SEQ ID NO: 55 - DNA, 336 bp, synthetic construct; identical to SEQ ID NO: 43
  source 1..336: mol_type = other DNA

SEQ ID NO: 56 - DNA, 357 bp, synthetic construct; identical to SEQ ID NO: 44
  source 1..357: mol_type = other DNA

SEQ ID NO: 57 - DNA, 336 bp, synthetic construct
  source 1..336: mol_type = other DNA
gatgtcgtgatgacccaaagccccctgtccctccctgtgactcctggagagccggcgtccatttcctgccggtcaagccagtccttggtggactacgacggaaagacctacctcaactggctgctgcagcgccccgggcagtcgccgcagcggcttatctacaaagtgtccaaccgcgactcgggcgtgccggataggttttcgggttccggaagcggcaccgacttcaccctgaaaatctccagagtggaagccgaggacgtgggagtgtactactgttggcagggttctcacttcccacggtcatatggccaagggactaaggtcgaaatcaag

SEQ ID NO: 58 - DNA, 357 bp, synthetic construct
  source 1..357: mol_type = other DNA
gaagtgcagctcctggagtccggcggtggactggtgcagccgggcggatccctgagactgtcctgcgccgcgtcgggctttactttcgcaaattacggcatgagctgggtcagacaggcccccgggaagggtctggaatgggtggccagcgtccggagcgggggatcccggacctattactccgacaacgtgaagggccgcttcaccatctcaagggacaactccaagaacaccctgtacttgcaaatgaacagccttcgggctgaggatactgccgtgtactactgcgtgcgctacgaccactactccggatcctcggattactggggacagggaaccctcgtgaccgtgtcatcg

SEQ ID NO: 59 - DNA, 336 bp, synthetic construct
  source 1..336: mol_type = other DNA
gatgtcgtgatgactcagtcgcccctctccctgcctgtgactctgggggaacccgcgtccatttcgtgcaagagcagccagtccctgatggacaccgacggaaagacctaccttaactggctgctgcaaaagccaggacagagcccgcagaggctgatctacaaagtgtcaaaccgggagtccggagtgccggacagattcagcggctcgggttccggcaccgacttcaccctcaaaatctcccgcgtcgaggccgaggacgtgggcgtgtattactgttggcagggaacccactttcctcggaccttcggtcaagggactaaggtcgaaatcaag

SEQ ID NO: 60 - DNA, 357 bp, synthetic construct; identical to SEQ ID NO: 44
  source 1..357: mol_type = other DNA

SEQ ID NO: 61 - DNA, 336 bp, synthetic construct
  source 1..336: mol_type = other DNA
gatgtcgtgatgactcagtcaccgctctccctccctgtgaccccgggcgaaccagcgtcgatctcctgcaagagcagccaatcattgctggactacgacggaaagacctatcttaactggctgctgcagaagcccgggcagagcccgcagcgcctgatctacaaagtgtccaacagagactccggagtgcctgataggttctcgggttccggctccggtaccgacttcactctgaaaatttcccgggtggaagccgaggacgtgggagtgtactactgttggcagggcacccacttcccccggtcgtttggacaagggaccaaggtcgagatcaag

SEQ ID NO: 62 - DNA, 357 bp, synthetic construct; identical to SEQ ID NO: 52
  source 1..357: mol_type = other DNA

SEQ ID NO: 63 - DNA, 336 bp, synthetic construct
  source 1..336: mol_type = other DNA
gatgtcgtgatgactcagtcgcccctctccctgcctgtgactctgggggaacccgcgtccatttcgtgcaagagcagccagtccctgttggactcagacggaaagacctaccttaactggctgctgcaaaagccaggacagagcccgcagaggctgatctacaaagtgtcaaaccgggattccggagtgccggacagattcagcggctcgggttccggcaccgacttcaccctcaaaatctcccgcgtcgaggccgaggacgtgggcgtgtattactgttggcagggaacccactttcctcggaccttcggtcaagggactaaggtcgaaatcaag

SEQ ID NO: 64 - DNA, 357 bp, synthetic construct; identical to SEQ ID NO: 52
  source 1..357: mol_type = other DNA

SEQ ID NO: 65 - DNA, 336 bp, synthetic construct
  source 1..336: mol_type = other DNA
gatgtcgtgatgactcagtcgcccctctccctgcctgtgactctgggggaacccgcgtccatttcgtgcaagagcagccagtccctgttggactcagacggaaagacctaccttaactggctgctgcaaaagccaggacagagcccgcagaggctgatctacaaagtgaccaaccgggagtccggagtgccggacagattcagcggctcgggttccggcaccgacttcaccctcaaaatctcccgcgtcgaggccgaggacgtgggcgtgtattactgttggcagggaacccactttcctcggtcattcggtcaagggactaaggtcgaaatcaag

SEQ ID NO: 66 - DNA, 357 bp, synthetic construct; identical to SEQ ID NO: 42
  source 1..357: mol_type = other DNA

SEQ ID NO: 67 - DNA, 336 bp, synthetic construct; identical to SEQ ID NO: 61
  source 1..336: mol_type = other DNA

SEQ ID NO: 68 - DNA, 357 bp, synthetic construct; identical to SEQ ID NO: 52
  source 1..357: mol_type = other DNA

SEQ ID NO: 69 - DNA, 336 bp, synthetic construct; identical to SEQ ID NO: 59
  source 1..336: mol_type = other DNA

SEQ ID NO: 70 - protein, 454 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 449 aa, synthetic construct
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 119 aa, synthetic construct [sequence omitted]

SEQ ID NO: 74-76 - skipped (3 entries)

SEQ ID NO: 77 - protein, 219 aa, synthetic construct
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 112 aa, synthetic construct [sequence omitted]

SEQ ID NO: 79-81 - skipped (3 entries)

SEQ ID NO: 82 - protein, 456 aa, synthetic construct
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 215 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 42 aa, Homo sapiens [sequence omitted]

SEQ ID NO: 85 - protein, 770 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 86 - DNA, 993 bp, synthetic construct
  source 1..993: mol_type = other DNA
gccagcactaaggggcctagcgtctttccgctggccccgtcctccaagtccacttcgggtggaaccgcggcactggggtgcctcgtgaaggactacttccccgagccggtcaccgtgtcctggaactcgggagccctgacctccggagtgcatactttccctgcggtgctgcagtcctccgggctctactcgctgtcaagcgtggtcaccgtcccgagctcatccctgggtactcagacctacatttgcaacgtgaaccacaaaccttccaacaccaaggtcgacaagaaagtggagcctaagagctgcgacaagacccacacctgtcccccgtgtcccgcccctgagctgctgggcggccccagcgtgttcctcttcccgcctaagccgaaggacactctgatgatctcgagaacccctgaagtgacctgtgtggtggtggatgtgtcccacgaggatccggaagtgaagttcaattggtacgtggacggagtggaagtccataacgccaagaccaagccccgcgaggaacagtacaactcaacttaccgggtggtgtcagtgctgaccgtgctgcaccaagattggctgaacgggaaggagtacaagtgcaaagtctccaacaaggcgctgccggcccccattgaaaagaccatcagcaaggctaagggccagccccgggaaccacaggtctacaccttgcccccttcccgggaggaaatgaccaagaaccaagtgtcgctgacgtgcctggtcaagggcttttatccatctgacatcgccgtggagtgggaaagcaacggccagccggaaaacaactacaagactaccccgcctgtgctggactccgacggctcgttcttcctgtattccaagctcaccgtggataagtccagatggcagcagggcaatgtgttcagctgcagcgtgatgcatgaggccctgcacaaccactacactcagaaatcactgtccctttcccccggaaagtaa

SEQ ID NO: 87 - DNA, 324 bp, synthetic construct
  source 1..324: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 88 - protein, 10 aa, synthetic construct
  VARIANT 7: note = X is Y or F
GFTFSNXGMS

SEQ ID NO: 89 - protein, 17 aa, synthetic construct
  VARIANT 2: note = X is I or V
SXRSGSGRTYYSDNVKG

SEQ ID NO: 90 - protein, 10 aa, synthetic construct
  VARIANT 5: note = X is S or T
  VARIANT 7: note = X is S or T
YDHYXGXSDY

SEQ ID NO: 91 - protein, 16 aa, synthetic construct
KSSQSLLDYDGKTYLN

SEQ ID NO: 92 - protein, 7 aa, Synthetic construct
  VARIANT 1: note = X is K or R
  VARIANT 3: note = X is S or T
  VARIANT 7: note = X is S or T
XVXNRDX

SEQ ID NO: 93 - protein, 9 aa, synthetic construct
  VARIANT 9: note = X is S or T
WQGTHFPRX

SEQ ID NO: 94 - protein, 11 aa, synthetic construct
  VARIANT 9: note = X is S or T
  VARIANT 11: note = X is F or Y
WQGTHFPRXFX

SEQ ID NO: 95 - protein, 10 aa, synthetic construct
  VARIANT 5: note = X is S or A
  VARIANT 7: note = X is Y or F
GFTFXNXGMS

SEQ ID NO: 96 - protein, 17 aa, synthetic construct
  VARIANT 2: note = X is I or V
  VARIANT 6: note = X is S or G
  VARIANT 7: note = X is S or G
SXRSGXXRTYYSDNVKG

SEQ ID NO: 97 - protein, 16 aa, synthetic construct
  VARIANT 1: note = X is K or R
  VARIANT 7: note = X is V, M, or L
  VARIANT 9: note = X is Y, T, or S
XSSQSLXDXDGKTYLN

SEQ ID NO: 98 - skipped

SEQ ID NO: 99 - protein, 9 aa, synthetic construct
  VARIANT 4: note = X is S or T
  VARIANT 9: note = X is S or T
WQGXHFPRX

SEQ ID NO: 100 - protein, 12 aa, synthetic construct
  VARIANT 9: note = X is S or T
  VARIANT 11: note = X is S or T
  VARIANT 12: note = X is F or Y
WQGTHFPRXFXX

SEQ ID NO: 101 - protein, 449 aa, synthetic construct
EVQLLESGGGLVQPGGSLRLSCAASGFTFSNYGMSWVRQAPGKGLEWVASIRSGSGRTYYSDNVKGRFTISRDNSKNTLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 219 aa, synthetic construct
DVVMTQSPLSLPVTLGEPASISCKSSQSLLDYDGKTYLNWLLQKPGQSPQRLIYRVTNRDTGVPDRFSGSGSGTDFTLKISRVEAEDVGVYYCWQGTHFPRSFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1347 bp, synthetic construct
  source 1..1347: mol_type = other DNA
gaggtgcagctcttggagtctgggggaggcttggtgcagccaggggggtccctaagactctcctgtgcagcctctggattcaccttctccaactatggcatgtcctgggtccgccaggctccagggaagggactggagtgggtcgcttctattcgctccggtagtggtaggacatactactcagataacgtgaagggccggttcacaatctccagagacaattccaagaacacgctgtatctgcaaatgaacagcctgagagccgaggatacggccgtttattactgtgtgcgctacgaccattactctggatcctctgactactggggccaaggcacccttgtcacagtctcctcagcctccaccaagggcccatcggtcttccccctggcaccctcttccaagagcacctctgggggcacagcggccctgggctgccttgtcaaggactacttccccgaaccggtgacggtgtcgtggaactcaggcgccctgaccagcggcgtgcacaccttcccggctgtcctacagtcctcaggactctactccctcagcagcgtggtgactgtgccctccagcagcttgggcacccagacctacatctgcaacgtgaatcacaagcccagcaacaccaaggtggataagaaggttgagcccaaatcttgtgacaaaactcacacatgcccaccgtgcccagcacctgaactcctggggggaccgtcagtcttcctgttccccccaaaacccaaggacaccctcatgatctcccggacccctgaggtcacatgcgtggtggtggacgtgagccacgaagacccagaggtcaagttcaactggtacgtggacggcgtggaggtgcataatgccaagacaaagccgagagaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcctccatcccgggaggagatgaccaagaaccaggtcagcctgacctgcctggtcaaaggcttctatcccagcgacatcgccgtcgagtgggagagcaatgggcagccggagaacaactacaagaccacgcctcccgtgctggactccgacggctccttcttcctctattccaagctcaccgtggacaagagcaggtggcagcaggggaacgtcttctcatgctccgtgatgcatgaggctctgcacaaccactacacgcagaagagcctctccctgtctcccgggaaa

SEQ ID NO: 104 - DNA, 657 bp, synthetic construct
  source 1..657: mol_type = other DNA
gatgttgtgatgacccagtccccactctctttgcccgttacccttggagaacctgcctccatctcttgcaagtcaagtcagagcctcttagattacgatggaaagacatatttgaattggttgctgcagaagccaggccagtctccacagcgcctaatctatcgggtgaccaaccgggacactggagtccctgacaggttcagtggcagtggatcagggacagatttcacactgaaaatcagcagagtggaggctgaggatgtgggagtttattattgctggcaaggcacacattttccgcgctctttcggacaggggaccaaggtggaaataaaacgaactgtggctgcaccatctgtcttcatcttcccgccatctgatgagcagcttaagtccggaactgctagcgttgtgtgcctgctgaataacttctatcccagagaggccaaagtacagtggaaggtggataacgccctccaatcgggaaactcccaggagagcgtcacagagcaggacagcaaagacagcacctacagcctcagcagcaccctgacgctgagcaaagcagactacgagaaacacaaagtctacgcctgcgaagtcacccatcagggcctgagctcgcccgtcacaaagagcttcaacaggggagagtgt